SEQUENCE LISTING (39 sequences)

SEQ ID NO: 1 - protein, 1048 aa, Bacillus megaterium
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 2 - protein, 1060 aa, Bacillus subtilis
KETSPIPQPKTFGPLGNLPLIDKDKPTLSLIKLAEEQGPIFQIHTPAGTTIVVSGHELVKEVCDEERFDKSIEGALEKVRAFSGDGLFTSWTHEPNWRKAHNILMPTFSQRAMKDYHEKMVDIAVQLIQKWARLNPNEAVDVPGDMTRLTLDTIGLCGFNYRFNSYYRETPHPFINSMVRALDEAMHQMQRLDVQDKLMVRTKRQFRYDIQTMFSLVDSIIAERRANGDQDEKDLLARMLNVEDPETGEKLDDENIRFQIITFLIAGHETTSGLLSFATYFLLKHPDKLKKAYEEVDRVLTDAAPTYKQVLELTYIRMILNESLRLWPTAPAFSLYPKEDTVIGGKFPITTNDRISVLIPQLHRDRDAWGKDAEEFRPERFEHQDQVPHHAYKPFGNGQRACIGMQFALHEATLVLGMILKYFTLIDHENYELDIKQTLTLKPGDFHISVQSRHQEAIHADVQAAEKAAPDEQKEKTEAKGASVIGLNNRPLLVLYGSDTGTAEGVARELADTASLHGVRTKTAPLNDRIGKLPKEGAVVIVTSSYNGKPPSNAGQFVQWLQEIKPGELEGVHYAVFGCGDHNWASTYQYVPRFIDEQLAEKGATRFSARGEGDVSGDFEGQLDEWKKSMWADAIKAFGLELNENADKERSTLSLQFVRGLGESPLARSYEASHASIAENRELQSADSDRSTRHIEIALPPDVEYQEGDHLGVLPKNSQTNVSRILHRFGLKGTDQVTLSASGRSAGHLPLGRPVSLHDLLSYSVEVQEAATRAQIRELASFTVCPPHRRELEELSAEGVYQEQILKKRISMLDLLEKYEACDMPFERFLELLRPLKPRYYSISSSPRVNPRQASITVGVVRGPAWSGRGEYRGVASNDLAERQAGDDVVMFIRTPESRFQLPKDPETPIIMVGPGTGVAPFRGFLQARDVLKREGKTLGEAHLYFGCRNDRDFIYRDELERFEKDGIVTVHTAFSRKEGMPKTYVQHLMADQADTLISILDRGGRLYVCGDGSKMAPDVEAALQKAYQAVHGTGEQEAQNWLRHLQDTGMYAKDVWAGI

SEQ ID NO: 3 - protein, 1053 aa, Bacillus subtilis
KQASAIPQPKTYGPLKNLPHLEKEQLSQSLWRIADELGPIFRFDFPGVSSVFVSGHNLVAEVCDEKRFDKNLGKGLQKVREFGGDGLFTSWTHEPNWQKAHRILLPSFSQKAMKGYHSMMLDIATQLIQKWSRLNPNEEIDVADDMTRLTLDTIGLCGFNYRFNSFYRDSQHPFITSMLRALKEAMNQSKRLGLQDKMMVKTKLQFQKDIEVMNSLVDRMIAERKANPDENIKDLLSLMLYAKDPVTGETLDDENIRYQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQADIKAETKPKETKPKHGTPLLVLFGSNLGTAEGIAGELAAQGRQMGFTAETAPLDDYIGKLPEEGAVVIVTASYNGAPPDNAAGFVEWLKELEEGQLKGVSYAVFGCGNRSWASTYQRIPRLIDDMMKAKGASRLTAIGEGDAADDFESHRESWENRFWKETMDAFDINEIAQKEDRPSLSITFLSEATETPVAKAYGAFEGIVLENRELQTAASTRSTRHIELEIPAGKTYKEGDHIGILPKNSRELVQRVLSRFGLQSNHVIKVSGSAHMAHLPMDRPIKVVDLLSSYVELQEPASRLQLRELASYTVCPPHQKELEQLVSDDGIYKEQVLAKRLTMLDFLEDYPACEMPFERFLALLPSLKPRYYSISSSPKVHANIVSMTVGVVKASAWSGRGEYRGVASNYLAELNTGDAAACFIRTPQSGFQMPNDPETPMIMVGPGTGIAPFRGFIQARSVLKKEGSTLGEALLYFGCRRPDHDDLYREELDQAEQDGLVTIRRCYSRVENEPKGYVQHLLKQDTQKLMTLIEKGAHIYVCGDGSQMAPDVERTLRLAYEAEKAASQEESAVWLQKLQDQRRYVKDVWTGM

SEQ ID NO: 4 - protein, 64 aa, Artificial Sequence
  Peptide sequence from P450 BM3
TIKEMPQPKTFGELKNLPLLNTDKPVQALMKIADELGEIFKFEAPGRVTRYLSSQRLIKEACDE

SEQ ID NO: 5 - protein, 65 aa, Artificial Sequence
  Peptide fragment of P450BM3
KETSPIPQPKTFGPLGNLPLIDKDKPTLSLIKLAEEQGPIFQIHTPAGTTIVVSGHELVKEVCDE

SEQ ID NO: 6 - protein, 65 aa, Artificial Sequence
  Peptide fragment form P40 BM3
KQASAIPQPKTYGPLKNLPHLEKEQLSQSLWRIADELGPIFRFDFPGVSSVFVSGHNLVAEVCDE

SEQ ID NO: 7 - protein, 58 aa, Artificial Sequence
  Peptide fragment of P450 BM3
SRFDKNLSQALKFVRDFAGDGLATSWTHEKNWKKAHNILLPSFSQQAMKGYHAMMVDI

SEQ ID NO: 8 - protein, 58 aa, Artificial Sequence
  Peptide fragment of P450 BM3
ERFDKSIEGALEKVRAFSGDGLATSWTHEPNWRKAHNILMPTFSQRAMKDYHEKMVDI

SEQ ID NO: 9 - protein, 58 aa, Artificial Sequence
  Peptide fragment of P450 BM3
KRFDKNLGKGLQKVREFGGDGLATSWTHEPNWQKAHRILLPSFSQKAMKGYHSMMLDI

SEQ ID NO: 10 - protein, 44 aa, Artificial Sequence
  Peptide fragment of P450 BM3
AVQLVQKWERLNADEHIEVPEDMTRLTLDTIGLCGFNYRFNSFY

SEQ ID NO: 11 - protein, 44 aa, Artificial Sequence
  Peptide fragment of P450 BM3
AVQLIQKWARLNPNEAVDVPGDMTRLTLDTIGLCGFNYRFNSYY

SEQ ID NO: 12 - protein, 44 aa, Artificial Sequene
  Peptide fragment of P450 BM3
ATQLIQKWSRLNPNEEIDVADDMTRLTLDTIGLCGFNYRFNSFY

SEQ ID NO: 13 - protein, 50 aa, Artificial Sequence
  Peptide fragment of P450 BM3
RDQPHPFITSMVRALDEAMNKLQRANPDDPAYDENKRQFQEDIKVMNDLV

SEQ ID NO: 14 - protein, 50 aa, Artificial Sequence
  Peptide fragment of P450 BM3
RETPHPFINSMVRALDEAMHQMQRLDVQDKLMVRTKRQFRYDIQTMFSLV

SEQ ID NO: 15 - protein, 50 aa, Artificial Sequence
  Peptide fragment of P450
RDSQHPFITSMLRALKEAMNQSKRLGLQDKMMVKTKLQFQKDIEVMNSLV

SEQ ID NO: 16 - protein, 52 aa, Artificial Sequence
  Peptide fragment of P450
DKIIADRKASGEQSDDLLTHMLNGKDPETGEPLDDENIRYQIITFLIAGHET

SEQ ID NO: 17 - protein, 53 aa, Artificial Sequence
  Peptide fragment of P450
DSIIAERRANGDQDEKDLLARMLNVEDPETGEKLDDENIRFQIITFLIAGHET

SEQ ID NO: 18 - protein, 53 aa, Artificial Sequence
  Peptide fragment of P450
DRMIAERKANPDENIKDLLSLMLYAKDPVTGETLDDENIRYQIITFLIAGHET

SEQ ID NO: 19 - protein, 61 aa, Artificial Sequence
  Peptide fragment of P450
TSGLLSFALYFLVKNPHVLQKAAEEAARVLVDPVPSYKQVKQLKYVGMVLNEALRLWPTAA

SEQ ID NO: 20 - protein, 60 aa, Artificial Sequence
  Peptide fragment of P450
TSGLLSFATYFLLKHPDKLKKAYEEVDRVLTDAAPTYKQVLELTYIRMILNESLRLWPTA

SEQ ID NO: 21 - protein, 60 aa, Artificial Sequence
  Peptide fragment of P450
TSGLLSFAIYCLLTHPEKLKKAQEEADRVLTDDTPEYKQIQQLKYIRMVLNETLRLYPTA

SEQ ID NO: 22 - protein, 76 aa, Artificial Sequence
  Peptide fragment of P450
PAFSLYAKEDTVLGGEYPLEKGDELMVLIPQLHRDKTIWGDDVEEFRPERFENPSAIPQHAFKPFGNGQRACIGQQ

SEQ ID NO: 23 - protein, 76 aa, Artificial Sequence
  Peptide fragment of P450
PAFSLYPKEDTVIGGKFPITTNDRISVLIPQLHRDRDAWGKDAEEFRPERFEHQDQVPHHAYKPFGNGQRACIGMQ

SEQ ID NO: 24 - protein, 76 aa, Artificial Sequence
  Peptide fragment of P450
PAFSLYAKEDTVLGGEYPISKGQPVTVLIPKLHRDQNAWGPDAEDFRPERFEDPSSIPHHAYKPFGNGQRACIGMQ

SEQ ID NO: 25 - protein, 59 aa, Artificial Sequence
  Peptide fragment of P450
FALHEATLVLGMMLKHFDFEDHTNYELDIKETLTLKPEGFVVKAKSKKIPLGGIPSPST

SEQ ID NO: 26 - protein, 60 aa, Artificial Sequence
  Peptide fragment of P450
FALHEATLVLGMILKYFTLIDHENYELDIKQTLTLKPGDFHISVQSRHQEAIHADVQAAE

SEQ ID NO: 27 - protein, 60 aa, Artificial Sequence
  Peptide fragment of P450
FALQEATMVLGLVLKHFELINHTGYELKIKEALTIKPDDFKITVKPRKTAAINVQRKEQA

SEQ ID NO: 28 - protein, 584 aa, Artificial Sequence
  Reductase domain from Bacillus sp.
EQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWA

SEQ ID NO: 29 - protein, 573 aa, Artificial Sequence
  Reductase domain from Bacillus sp.
ADNLSLLVLYGSDTGVAEGIARELADTASLEGVQTEVAALNDRIGSLPKEGAVLIVTSSYNGKPPSNAGQFVQWLEELKGDELKGVQYAVFGCGDHNWASTYQRIPRYIDEQMAQKGATRFSTRGEADASGDFEEQLEQWKESMWSDAMKAFGLELNKNIEKERSTLSLQFVSRLGGSPLARTYEAVYASILENRELQSSSSERSTRHIEISLPEGATYKEGDHLGVLPINSEKNVNRILKRFGLNGKDQVILSASGRSVNHIPLDSPVRLYDLLSYSVEVQEAATRAQIREMVTFTACPPHKKELESLLEDGVYHEQILKKRISMLDLLEKYEACEIRFERFLELLPALKPRYYSISSSPLVAQNRLSITVGVVNAPAWSGEGTYEGVASNYLAQLHNKDEIICFIRTPQSNFQLPENPETPIIMVGPGTGIAPFRGFLQARRVQKQKGMKVGEAHLYFGCRHPEKDYLYRTELENDERDGLISLHTAFSRLEGHPKTYVQHVIKQDRIHLISLLDNGAHFYICGDGSKMAPDVEDTLCQAYQEIHEVSEQEARNWLDRLQEEGRYGKDVWA

SEQ ID NO: 30 - protein, 573 aa, Artificial Sequence
  Reductase domain from Bacillus sp.
ADNLSLLVLYGSDTGVAEGIARELADTASLEGVQTEVVALNDRIGSLPKEGAVLIVTSSYNGKPPSNAGQFVQWLEELKPDELKGVQYAVFGCGDHNWASTYQRIPRYIDEQMAQKGATRFSKRGEADASGDFEEQLEQWKQGMWSDAMKAFGLEFNKNMEKERSTLSLQFVSRLGGSPLARTYEAVYATILENRELQSSSSDRSTRHIEVSLPEGATYQEGDHLGVLPINSEKNVNRILKRFGLNGKDQVILSASGRSINHIPLDSPVSLLDLLSYSVEVQEAATRAQIREMVTFTACPPHKKELEALLEEGVYHEQILKKRISMLDLLEKYEACEIRFERFLELLPALKPRYYSISSSPLVAQNRLSITVGVVNAPAWSGEGTYEGVASNYLAQRHNKDEIICFIRTPQSNFELPKDPETPIIMVGPGTGVAPFRGFLQARRVQKQKGINLGQAHLYFGCRHPEKDYLYRTELENDERDGLISLHTAFSRLEGHPKTYVQHLIKQDSINLISLLDNGAHLYICGDGSKMAPDVEDTLCQAYQEIHEVSEQEARNWLDRVQDEGRYGKDVWA

SEQ ID NO: 31 - protein, 573 aa, Artificial Sequence
  Reductase domain from Bacillus sp.
ADNLSLLVLYGSDTGVAEGIARELADTASLEGVQTEVAALNDRIGSLPKEGAVLIVTSSYNGKPPSNAGQFVQWLEELKPDELKGVQYAVFGCGDHNWASTYQRIPRYIDEQMAQKGATRFSKRGEADASGDFEEQLEQWKQSMWSDAMKAFGLELNKNMEKERSTLSLQFVSRLGGSPLARTYEAVYASILENRELQTSSSERSTRHIEVSLPEGATYKEGDHLGVLPINSEKNVNRILKRFGLNGKDQVILSASGRSVNHIPLDSPVRLYDLLSYSVEVQEAATRAQIREMVTFTVCPPHKKELESLLEEGVYQEQILKKRISMLDLLEKYEACEIRFERFLELLPALKPRYYSISSSPLVAQDRLSITVGVVNAPAWSGEGTYEGVASNYLAQRHNKDEIICFIRTPQSNFQLPENPETPIIMVGPGTGIAPFRGFLQARRVQKQKGMNLGEAHLYFGCRHPEKDYLYRTELENDEREGLISLHTAFSRLEGHPKTYVQHVIKEDRIHLISLLDNGAHLYICGDGSKMAPDVEDTLCQAYQEIHEVSEQEARNWLDRVQDEGRYGKDVWA

SEQ ID NO: 32 - protein, 573 aa, Artificial Sequence
  Reductase domain from Bacillus sp.
ADNLSLLVLYGSDTGVAEGIARELADTASLEGVQTEVVALNDRIGSLPKEGAVLIVTSSYNGKPPSNAGQFVQWLEELKPDELKGVQYAVFGCGDHNWASTYQRIPRYIDEQMAQKGATRFSKRGEADASGDFEEQLEQWKQSMWSDAMKAFGLELNKNMEKERSTLSLQFVSRLGGSPLARTYEAVYASILENRELQSSSSDRSTRHIEVSLPEGATYKEGDHLGVLPVNSEKNINRILKRFGLNGKDQVILSASGRSINHIPLDSPVSLLDLLSYSVEVQEAATRAQIREMVTFTACPPHKKELEALLEEGVYHEQILKKRISMLDLLEKYEACEIRFERFLELLPALKPRYYSISSSPLVAQNRLSITVGVVNAPAWSGEGTYEGVASNYLAQRHNKDEIICFIRTPQSNFELPKDPETPIIMVGPGTGIAPFRGFLQARRVQKQKGINLGEAHLYFGCRHPEKDYLYRTELENDERDGLISLHTAFSRLEGHPKTYVQHLIKQDRINLISLLDNGAHLYICGDGSKMAPDVEDTLCQAYQEIHEVSEQEARNWLDRVQDEGRYGKDVWA

SEQ ID NO: 33 - protein, 573 aa, Artificial Sequence
  Reductase domain from Bacillus sp.
ADNLSLLVLYGSDTGVAEGIARELADTASLEGVRTEVVALNDQIGSLPKEGAVLIVTSSYNGKPPSNAGQFVQWLEELKPDELKGVQYAVFGCGDHNWASTYQRIPRYIDEQMAQKGATRFSKRGEADASGDFEEQLEQWKQSMWSDAMKAFGLELNKNMEKERSTLSLQFVSRLGGSPLARTYEAVYASILENRELQSSSSDRSTRHIEVSLPEGATYKEGDHLGVLPVNSEKNINRILKRFGLNGKDQVILSASGRSINHIPLDSPVSLLDLLSYSVEVQEAATRAQIREMVTFTACPPHKKELEALLEEGVYHEQILKKRISMLDLLEKYEACEIRFERFLELLPALKPRYYSISSSPLVAHNRLSITVGVVNAPAWSGEGTYEGVASNYLAQRHNKDEIICFIRTPQSNFELPKDPETPIIMVGPGTGIAPFRGFLQARRVQKQKGMNLGQAHLYFGCRHPEKDYLYRTELENDERDGLISLHTAFSRLEGHPKTYVQHLIKQDRINLISLLDNGAHLYICGDGSKMAPDVEDTLCQAYQEIHEVSEQEARNWLDRVQDEGRYGKDVWA

SEQ ID NO: 34 - protein, 573 aa, Artificial Sequence
  Reductase domain from Bacillus sp.
ADNLSLLVLYGSDTGVAEGIARELADTASLEGVQTEVAALNDRIGSLPKEGAVLIVTSSYNGKPPSNAGQFVQWLEELKPDELKGVQYAVFGCGDHNWASTYQRIPRYIDEQMAQKGATRFSTRGEADASGDFEEQLEQWKESMWSDAMKAFGLELNKNMEKERSTLSLQFVSRLGGSPLARTYEAVYASILENRELQSSSSERSTRHIEISLPEGATYKEGDHLGVLPINSEKNVNRILKRFGLNGKDQVILSASGRSVNHIPLDSPVRLYDLLSYSVEVQEAATRAQIREMVTFTACPPHKKELESLLEDGVYHEQILKKRISMLDLLEKYEACEIRFERFLELLPALKPRYYSISSSPLIAQDRLSITVGVVNAPAWSGEGTYEGVASNYLAQRHNKDEIICFIRTPQSNFQLPENPETPIIMVGPGTGIAPFRGFLQARRVQKQKGMNLGEAHLYFGCRHPEKDYLYRTELENDERDGLISLHTAFSRLEGHPKTYVQHVIKEDRMNLISLLDNGAHLYICGDGSKMAPDVEDTLCQAYQEIHEVSEQEARNWLDRLQDEGRYGKDVWA

SEQ ID NO: 35 - protein, 573 aa, Artificial Sequence
  Reductase domain from Bacillus sp.
ADNLSLLVLYGSDTGVAEGIARELADTASLEGVQTEVVALNDRIGSLPKEGAVLIVTSSYNGKPPSNAGQFVQWLEELKPDELKGVQYAVFGCGDHNWASTYQRIPRYIDEQMAQKGATRFSKRGEADASGDFEEQLEQWKQNMWSDAMKAFGLELNKNMEKERSTLSLQFVSRLGGSPLARTYEAVYASILENRELQSSSSDRSTRHIEVSLPEGATYKEGDHLGVLPVNSEKNINRILKRFGLNGKDQVILSASGRSINHIPLDSPVSLLALLSYSVEVQEAATRAQIREMVTFTACPPHKKELEALLEEGVYHEQILKKRISMLDLLEKYEACEIRFERFLELLPALKPRYYSISSSPLVAHNRLSITVGVVNAPAWSGEGTYEGVASNYLAQRHNKDEIICFIRTPQSNFELPKDPETPIIMVGPGTGIAPFRGFLQARRVQKQKGMNLGQAHLYFGCRHPEKDYLYRTELENDERDGLISLHTAFSRLEGHPKTYVQHLIKQDRINLISLLDNGAHLYICGDGSKMAPDVEDTLCQAYQEIHEVSEQEARNWLDRVQDEGRYGKDVWA

SEQ ID NO: 36 - protein, 569 aa, Artificial Sequence
  Reductase domain from Bacillus sp.
SHGTPLLVLYGSNLGTAQQIANELAEDGKAKGFDMTTAPLDDYARQLPDKGAVLIVTASYNGHPPDHAKTFVDWVTQDKEKDLTNVTFAVFGCGDRNWASTYQRIPRLIDEALESKGAKRVADLGEGDAGGDMDEDKETFQKIVFEQLAKEFQLTFQEKGKETPKLSVAYTNELVERPVAKTYGAFSAVVLKNEELQSQKSERKTRHIELRLPEGKKYKEGDHIGIVPKNRDVLVQRVIDRFNLDPKQHIKLSSEKEANHLPLGQPIQIRELLASHVELQEPATRTQLRELASYTVCPPHRVELEQMAGEAYQEAILKKRVTMLDLLDQYEACEMPFAHFLALLPGLKPRYYSISSSPKIDEKRVSITVAVVKGKAWSGRGEYAGVASNYLCDLQKGEEVACFLHEAQAGFQLPPSSETPMIMIGPGTGIAPFRGFVQAREVWQKEGKRLGEAHLYFGCRHPHEDDLYFEEMQLAAQKGVVHIRRAYSRHKDQKVYVQHLLKEDGGMLIKLLDEGAYLYVCGDGKVMAPDVESTLIDLYQHEKQCSKEDAENWLTTLANNNRYVKDVWS

SEQ ID NO: 37 - DNA, 3150 bp, Bacillus megaterium
atgacaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttatttacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 38 - DNA, 3186 bp, Bacillus subtilis
atgaaggaaacaagcccgattcctcagccgaagacgtttgggccgctcggcaatttgcctttaattgataaagacaaaccgacgctttcgctgatcaaactggcggaagaacagggcccgatttttcaaatccatacacccgcgggcacgaccattgtagtgtccggccatgaattggtgaaagaggtttgtgatgaagaacggtttgataaaagcattgaaggcgccttggaaaaggttcgcgcattttccggtgacggattgtttacgagctggacgcatgagcctaactggagaaaagcgcacaacattctgatgccgacgttcagccagcgggccatgaaggactatcatgagaaaatggtcgatatcgctgttcagctcattcaaaaatgggcaaggctcaacccgaatgaagcagtcgatgtcccgggagatatgacccggctgacgctcgacaccattgggctatgcgggtttaactaccgctttaacagttactacagagaaacgccccacccgtttatcaacagcatggtgcgggcgcttgatgaagcgatgcatcaaatgcagcggcttgatgttcaagataagcttatggtcagaacaaagcggcaattccgctatgatattcaaacgatgttttcgttagtcgacagcattattgcagagcgcagggcgaatggagaccaggatgaaaaagatttgctcgcccgcatgctgaatgtggaagatccggaaactggtgaaaagctcgacgacgaaaatatccgctttcagatcatcacgtttttgattgccggccatgaaacaacgagcggcctgctttcctttgcgacttactttttattgaagcatcctgacaaactgaaaaaggcgtatgaagaggtcgatcgggtgctgacggatgcagcgccgacctataaacaagtgctggagcttacatacatacggatgattttaaatgaatcactgcgcttatggccgacagctccggctttcagcctttatccaaaagaagacacagtcattggcggaaaatttccgatcacgacgaatgacagaatttctgtgctgattccgcagcttcatcgtgatcgagacgcttggggaaaggacgcagaagaattccggccggaacggtttgagcatcaggaccaagtgcctcatcatgcgtacaaaccattcggaaatggacaacgggcctgtatcggcatgcagtttgcccttcatgaagccacacttgtgttaggcatgattctaaaatatttcacattgattgatcatgagaattatgagcttgatatcaaacaaaccttaacacttaagccgggcgattttcacatcagtgttcaaagccgtcatcaggaagccattcatgcagacgtccaggcagctgaaaaagccgcgcctgatgagcaaaaggagaaaacggaagcaaagggtgcatcggtcatcggtcttaacaaccgcccgcttctcgtgctgtacggctcagataccggcaccgcagaaggcgtcgcccgggagcttgctgatactgccagtcttcacggcgtaaggacaaagacagcacctctgaacgaccggattggaaagctgccgaaagagggagcggttgtcattgtgacctcgtcttataatggaaagccgccaagcaatgccggacaattcgtgcagtggcttcaagaaatcaaaccgggtgagcttgagggcgtccattacgcggtatttggctgcggcgaccacaactgggcgagcacgtatcaatacgtgccgagattcattgatgagcagcttgcggagaaaggcgcgactcggttttctgcgcgcggggaaggggatgtgagcggtgattttgaagggcagcttgacgagtggaaaaaaagcatgtgggcggatgccatcaaagcattcggacttgagcttaatgaaaacgctgataaggaacgaagcacgctgagccttcagtttgtcagagggctgggcgagtctccgctcgctagatcgtacgaagcctctcacgcatccattgccgaaaatcgtgaactccagtccgcagacagcgatcgaagcactcgccatatcgaaattgcattgccgccggatgttgaatatcaagagggcgaccatcttggcgtattgccaaaaaacagccaaaccaatgtcagccggattcttcacagattcggtctgaagggaaccgaccaagtgacattgtcggcaagcggccgcagtgcggggcatctgccattgggccgtcctgtcagcctgcatgatcttctcagctacagcgtcgaggtgcaggaagcagccacaagagcgcaaatacgtgaactggcgtcatttacagtgtgtccgccgcataggcgcgaattagaagaactgtcagcagagggtgtttatcaggagcaaatattgaaaaaacgaatttccatgctggatctgcttgaaaagtatgaagcgtgtgacatgccgtttgaacgatttttagagcttttacggccgttaaaaccgagatactattcgatttcaagctctccaagagtgaatccgcggcaagcatcgatcacagtcggtgtcgtgcgcggcccggcgtggagcggccgtggcgaatacaggggtgtggcatcaaatgatttagctgagcgtcaagccggtgatgatgtcgtgatgtttatccgcacaccggaatcccggtttcagcttccgaaagaccctgaaacgccaattattatggtcgggccaggcacgggagtcgcgccatttcgcggtttccttcaagcccgcgatgttttaaagcgggagggcaaaacgctcggtgaggctcatctctattttggatgcaggaacgatcgggattttatttaccgagatgagcttgagcggtttgaaaaagacggaatcgtcactgtccacacagccttttcccgaaaagagggcatgccgaaaacatatgtccagcatctcatggctgaccaagcagatacattaatatcaatccttgaccgcggtggcaggctttatgtatgcggtgatggcagcaaaatggccccggatgtggaggcggcacttcaaaaagcgtatcaggctgtccatggaaccggggaacaagaagcgcaaaactggctgagacatctgcaggataccggtatgtacgctaaggatgtctgggcagggatatag

SEQ ID NO: 39 - DNA, 3165 bp, Bacillus subtilis
ttacattcctgtccaaacgtctttcacataacgtctttgatcttgcagcttttgcagccatacagctgattcttcctgacttgctgctttttcagcttcatatgccaatcgcaaagttctctctacatcaggagccatttgcgatccatcaccgcatacgtaaatatgagcccctttttcaatgagtgtcatcaatttctgcgtatcttgcttgagcaagtgctggacatatccttttggttcgttttcgacgcgcgagtagcatcggcggattgtgaccaaaccgtcctgttccgcttgatccagctcttctctgtaaaggtcgtcatggtccgggcggcggcagccgaagtataaaagtgcttcaccaagggtgcttccttccttcttcaaaaccgatcttgcctgaataaagcctctgaatggcgcaattcctgtgcccggcccgaccataatcataggcgtttcaggatcattcggcatctgaaatccggactgcggcgtacgaatgaagcaagctgctgcatcacctgtattcaattctgctaaataattagaggcgacaccccggtattcacctcggccgctccatgctgaggctttcacaactcctaccgtcatgctcacgatatttgcatgaactttcggtgagcttgaaatggaatagtatctcggttttagtgatggcaaaagtgctaaaaaccgttcaaacggcatttcgcaagcaggataatcctctaaaaaatcaagcatggtaagacgttttgcaagtacctgctctttgtaaatgccatcatctgaaacgagctgttccagctctttttgatgcggcggacaaactgtataagaggccagctcccgaagctgaagccttgatgccggttcctgcagctctacataggacgacaataaatccactactttgattggccgatccatcggcagatgagccatatgagcgcttccgcttacttttatcacatgattggactgcaaaccgaatcggctgagaacccgctgaacaagctccctgctgttctttggcaggattccgatatgatcgccttctttatatgttttaccagccggaatttccaattcaatatggcgggttgaacgcgtgctggcagctgtctggagttctcgattctctaacacaatcccttcaaacgcgccatatgctttagcaaccggcgtttccgtcgcttcactgagaaaagtaatcgataatgaaggcctgtcttctttctgggctatttcgttaatatcaaatgcgtccatcgtttccttccagaagcggttttcccaagactcgcggtggctttcaaaatcatcggcggcgtcaccttccccaatcgctgttaaacgcgatgccccctttgctttcatcatgtcatcaatcaggcggggaatccgctgatacgtgctggcccagctccggtttccgcagccgaataccgcataggaaacacctttcaattggccttcctcaagctctttcagccactctacaaatccggcagcattatcaggcggcgccccattataagaagccgttacaatgacgactgccccttcttcagggagcttgccgatataatcatcaagcggagccgtttcagctgtaaagcccatctggcggccttgagcagccagttcaccggctattccctcagctgtcccaagatttgaaccaaaaagaacaagtaaaggtgtgccgtgtttaggtttggtttcttttggctttgtttctgctttgatgtctgcctgttcttttctctgtacattgattgccgctgtttttcgcggtttcacagtaattttaaaatcatccggcttgatcgttaatgcttctttgatttttagttcgtagccagtatggtttatcaattcaaaatgctttaatacaagaccgagaaccattgtcgcttcttgaagagcaaactgcatgccaatacaagcgcgctgtccgtttccaaacggcttatacgcatggtgagggatacttgaaggatcctcaaaccgttccggacggaaatcttccgcatccggtccccaagcgttttgatcccggtgcagttttggaattaaaacagtgactggctgccctttgctgatcggatattccccgcctagaacagtatcctccttcgcatatagagaaaaagccggagctgttggatacagtctgagggtttcatttaaaaccatccgaatgtatttgagctgctggatttgtttatattcaggcgtgtcatccgttaacacgcgatccgcttcctcctgagcttttttcagtttttccggatgtgtaagcagacaataaatcgcaaaggatagcaacccgcttgttgtctcatgtccagcaattaaaaatgtgatgatttggtatcgaatgttttcgtcatccagcgtttcacccgttactggatctttggcataaagcatgagagacaagagatccttaatgttttcatccggattcgcctttcgctccgctatcattctatcaaccagggagttcatgacttctatatccttttggaactgcagcttcgttttcaccatcattttatcttgcaggcccagtcttttcgattgattcatcgcctcttttaaggcacggagcatactggtgataaacggatgctgtgaatcacggtaaaagctgttgaatcgatagttaaacccgcataacccaatcgtatcaagcgtcagacgtgtcatatcgtccgctacatcaatttcttcattagggtttaaccggctccacttttgaatcagctgggttgcgatatccagcatcatagaatgatagcctttcatcgctttttgactaaaactcggcagcaaaatgcggtgggctttttgccagttcggttcgtgcgtccagcttgtaaataagccatctcccccgaactcacgcaccttttgcaagcctttgccaaggttcttgtcaaagcgtttttcatcacacacttcagccacaagattgtggccggacacaaaaacactggatactcccggaaaatcaaaacggaaaatcggtcccaattcatcagctatccgccataaggattgagaaagctgttctttttccagatgcggaagattttttaaaggtccgtatgttttgggctgaggtattgcgcttgcctgtttcat

(19) United States
(12) Patent Application Publication
(10) Pub. No.: US 2008/0268517 A1
(43) Pub. Date: Oct. 30, 2008

(54) STABLE, FUNCTIONAL CHIMERIC CYTOCHROME P450 HOLOENZYMES

(75) Inventors: Frances H. Arnold, Pasadena, CA (US); Yougen Li, Lawrenceville, NJ (US)

Correspondence Address: BUCHANAN, INGERSOLL & ROONEY LLP, P.O. BOX 1404, ALEXANDRIA, VA, 22313-1404, US

(73) Assignee: THE CALIFORNIA INSTITUTE OF TECHNOLOGY, Pasadena, CA (US)

(21) Appl. No.: 12/049,318

(22) Filed: Mar. 15, 2008

Related U.S. Application Data
(63) Continuation-in-part of application No. 12/024,515, filed on Feb. 1, 2008.
Application No. 12/024,515 is a continuation-in-part of application No. 12/027,885, filed on Feb. 7, 2008.
(60) Provisional application No. 60/918,528, filed on Mar. 16, 2007.
(60) Provisional application No. 60/900,229, filed on Feb. 8, 2007.

(51) Int. Cl. C12N 9/02; C12N 15/11; C12N 1/20; C12N 15/00

(52) U.S. Cl. 435/189; 536/23.2; 435/320.1; 435/252.33

(57) ABSTRACT

The present disclosure relates to cytochrome p450 fusion polypeptides, nucleic acids encoding the polypeptides, and host cells for producing the polypeptides.

### CROSS REFERENCE TO RELATED APPLICATIONS

The application claims priority under 35 U.S.C. §119 to U.S. Provisional Application Ser. No. 60/918,528, filed, Mar. 16, 2007, the application also claims priority to U.S. patent application Ser. No. 12/024,515, filed Feb. 1, 2008, and U.S. patent application Ser. No. 12/027,885, filed Feb. 7, 2008, the disclosures of which are incorporated herein by reference.

### STATEMENT REGARDING FEDERALLY SPONSORED RESEARCH

The U.S. Government has certain rights in this invention pursuant to Grant No. GM068664 awarded by the National Institutes of Health and Grant No. DAAD19-03-0D-0004 awarded by ARO-US Army Robert Morris Acquisition Center.

### TECHNICAL FIELD

The present disclosure relates to biomolecular engineering and design, and engineered proteins and nucleic acids.

### BACKGROUND

Cytochrome p450 enzymes are a diverse superfamily of heme proteins that can act of a variety of exogenous and endogenous substrates, including alkanes and complex organic molecules, such as steroids and fatty acids. These enzymes catalyze a monooxygenase reaction in which an oxygen atom is inserted into an unactivated C—H bond. Cytochrome p450 enzymes metabolize many drug compounds, including transformation to their active metabolites, and therefore can affect a drug's efficacy, toxicity, and pharmacokinetic profile. In addition, cytochrome p450 enzymes in bacteria and other microorganisms can process toxic organic compounds, thereby offering avenues for removal or detoxification of environmental toxins and organic pollutants. Thus, it is desirable to identify cytochrome p450 enzymes having different substrate activity profiles as well as improvements in enzyme properties.

### SUMMARY

In one aspect, the present disclosure provides cytochrome p450 enzymes having chimeric heme domains fused to reductases domains. These polypeptides are shown to display different substrate specificities as well as changes in other enzyme properties, such as enzyme activity, as compared to the parent enzymes or the non-chimeric heme domains fused to the cytochrome p450 reductase domains. The chimeric heme domains are based on use of structure guided recombination (SCHEMA) to minimize structural perturbations to the polypeptide structure.

In another aspect, the disclosure also provides polynucleotides encoding the fusion polypeptides. The polynucleotide may be contained in a vector, or within the genome of a host cell and used to express the polypeptides.

In a further aspect, the disclosure provides the polypeptides in various compositions, such as a purified preparation comprising from about 40-100% purity of a polypeptide. The polypeptide can also be in the form of whole cell preparations or powder preparations. In some embodiments, the enzyme preparation is used in the producing a product wherein a substrate is contacted with a polypeptide of the disclosure to convert the substrate to the desired product.

### BRIEF DESCRIPTION OF THE FIGURES

FIG. 1 depicts recombination points and the sequence domains used to generate exemplary chimeric heme domains of the engineered cytochrome p450 enzymes.

FIG. 2 shows the amino acid sequence for CYP102A1 (SEQ ID NO:1).

FIG. 3 shows the amino acid sequence for CYP102A2 (SEQ ID NO:2).

FIG. 4 shows the amino acid sequence for CYP102A3 (SEQ ID NO:3).

FIGS. 5A and 5B show an alignment of SEQ ID NOs:1-3.

FIG. 6 shows chemical structures of substrates used to examine the specificity of the cytochrome p450 enzymes. Substrates are grouped according to the pairwise correlations. Members of a group are highly correlated; intergroup correlations are low.

FIG. 7 shows a summary of normalized activities for 56 enzymes acting on 11 substrates. Activities are shown using a color scale (white indicating highest and black lowest activity), with columns representing substrates and rows representing proteins. A3, A3-R1 and A3-R2 proteins, which were not analyzed, are shown in grey. Protein rows are ordered by their chimeric sequence first, and then by heme domain (R0) and R1, R2- and R3-fusions.

FIG. 8(A to D) shows substrate-activity profiles for parent heme domain mono- and peroxygenases. Panel (A) shows parent peroxygenases, panel (B) parent holoenzyme monooxygenases profiles, panel (C) the A1 protein set and panel (D) the A2 protein set. In (A) and (B) the origin of the heme domain (A1(“1”)l A2(“2”) and A3(“3”)). The protein set in panel (C) includes the heme domain A1 or its R1-, R2- or R3-fusion protein. Panel (D) depicts the A2 protein set.

FIG. 9(A to F) shows K-means clustering analysis separates chimeras into five clusters. All protein-activity profiles are depicted in (A). Panels (B) through (F) show profiles for sequences within each cluster. Panel (B) depicts 32312333-R1/R2, 32313233-R1/R2. Panel (C) depicts 22213132-R2, 21313111-R3, 21313311-R3. Panel (D) depicts A1-R1/R2, 12112333-R1/R2, 11113311-R1/R2 and 22213132-R1. Panel (E) depicts 21313111-R1/R2, 22313233-R2, 22312333-R2, 32312231-R2, 32312333-R0, 32312333-R3, 32313233-R0, and 32313233-R3. Panel (F) depicts the remaining sequences.

FIG. 10(A to P) shows substrate-activity profiles of the indicated chimeras. The columns are coded as follows from front to back: heme domain (R0, front), R1-, R2-, R3-fusion protein.

FIGS. 11(A and B) are examples of the correlation of absorbances values measured within substrate Group A and Group B. Panel (A) shows the correlation between diphenyl ether (DP) and ethyl phenoxyacetate (PA) with a R2=0.71. Panel (B) shows the correlation between tolbutamide (TB) activity and chlorzoxazone CH) activity with R2=0.94.

FIGS. 12A, 12B, 12C, 12D, and 12E provide sequences of reductase domains. SEQ ID NOs: 36-43 are greater than 50% identical to SEQ ID NO:35. The figure also provides polynucleotide sequences (SEQ ID NO:44-46) encoding polypeptides of SEQ ID NOs:1, 2, and 3 respectively.

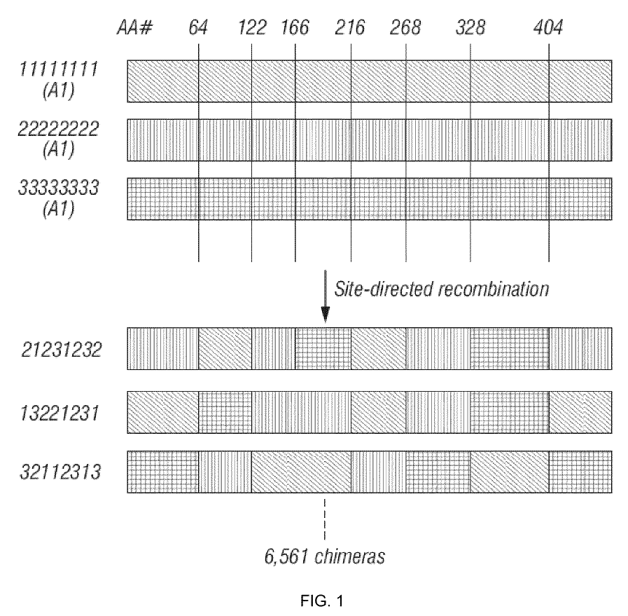

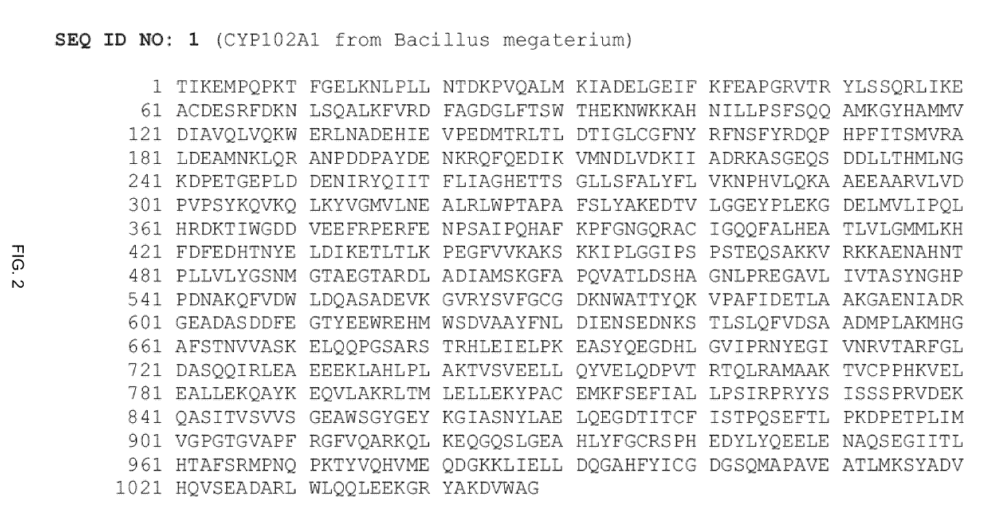

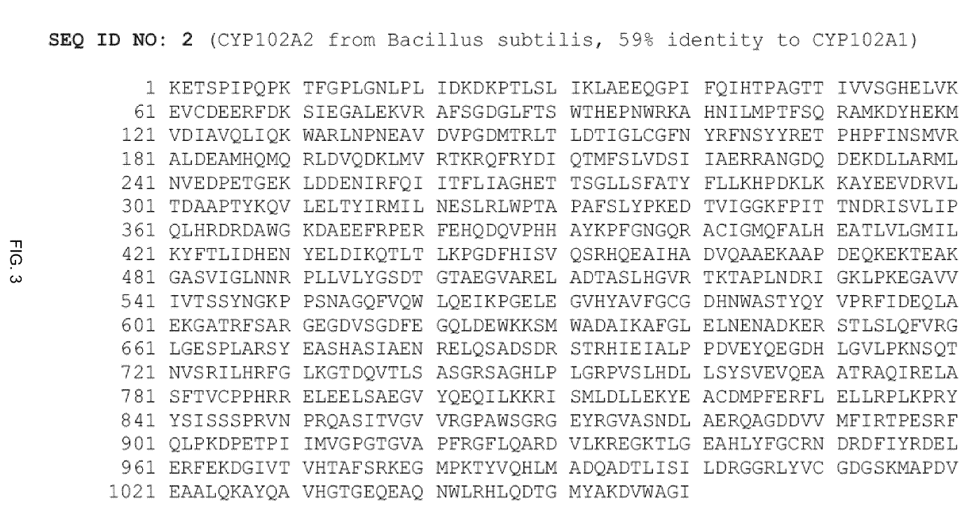

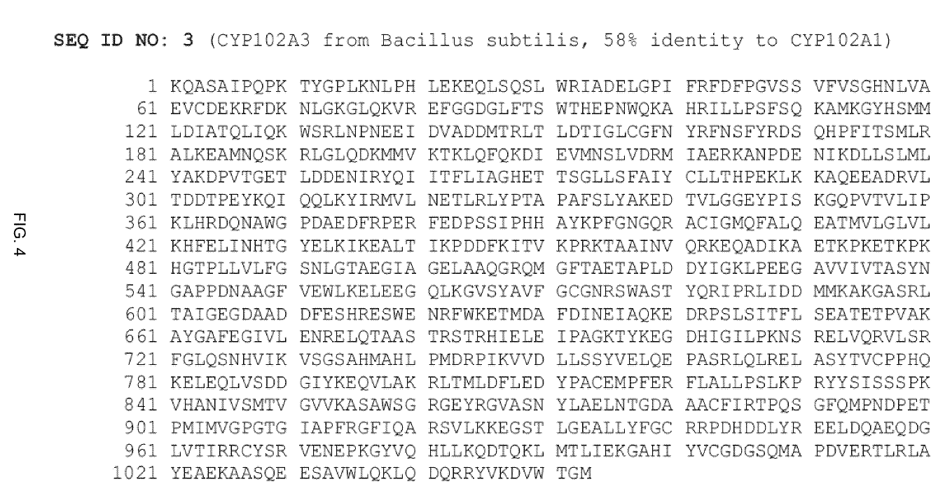

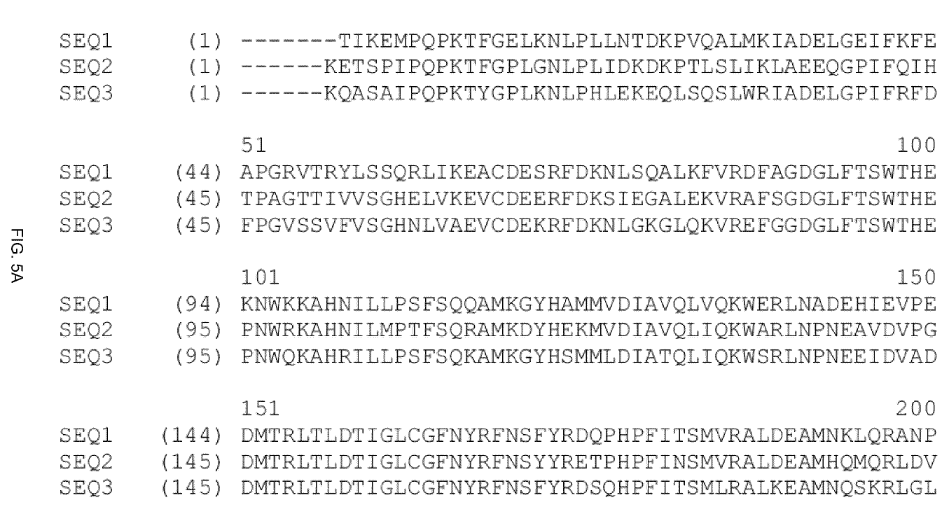

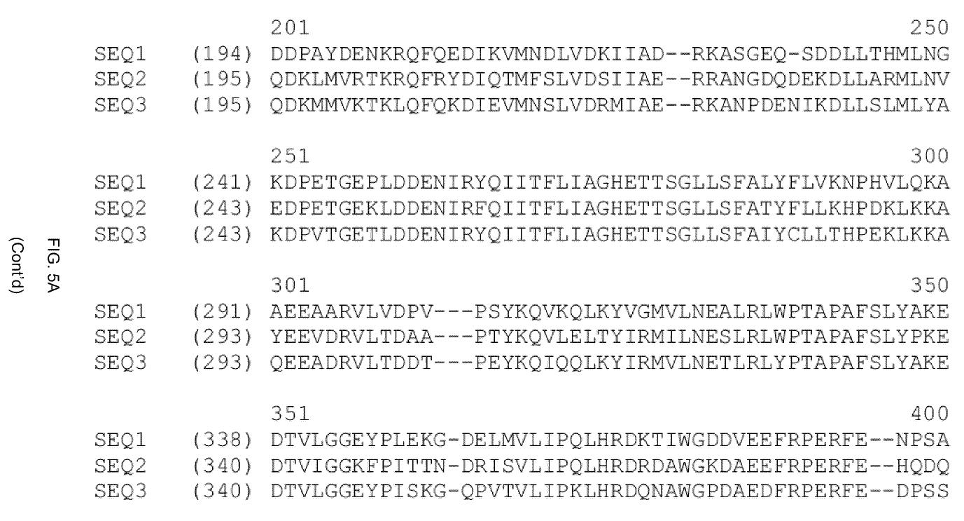

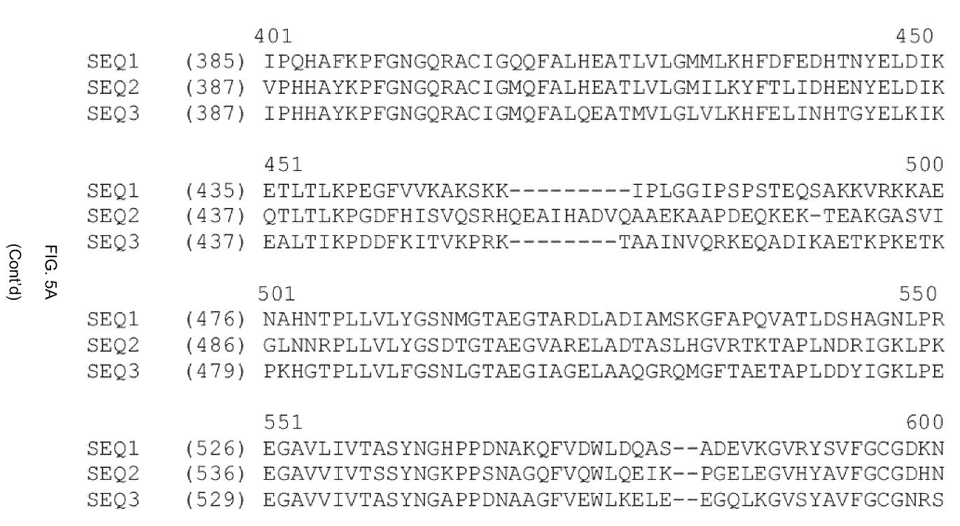

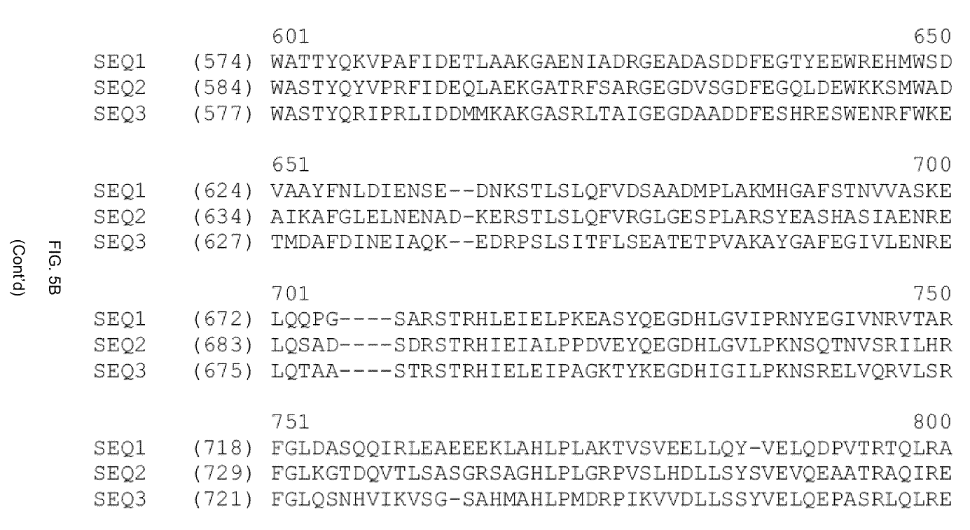

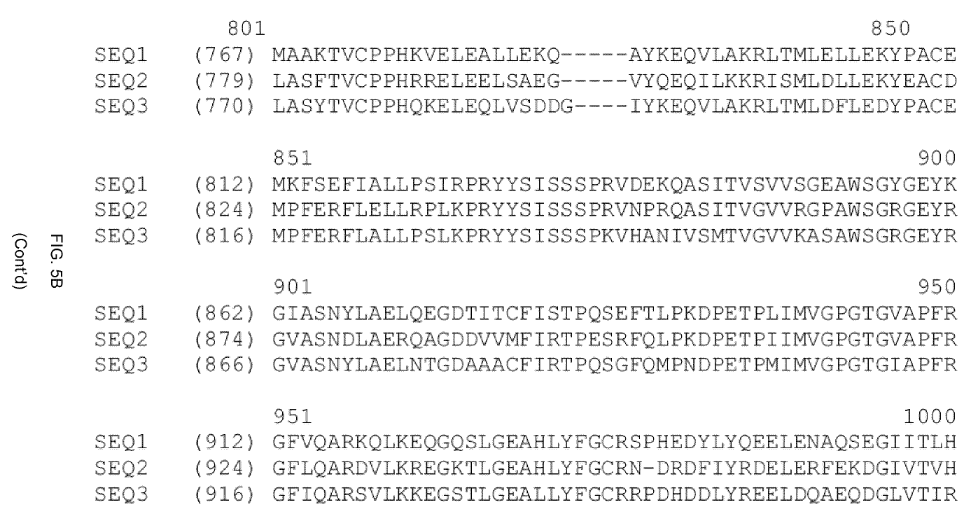

### DETAILED DESCRIPTION

As used herein and in the appended claims, the singular forms “a,” “and,” and “the” include plural referents unless the context clearly dictates otherwise. Thus, for example, reference to “a domain” includes a plurality of such domains and reference to “the protein” includes reference to one or more proteins, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Although methods and materials similar or equivalent to those described herein can be used in the practice of the disclosed methods and compositions, the exemplary methods, devices and materials are described herein.

Unless defined otherwise, all technical and scientific terms used herein have the same meaning as commonly understood to one of ordinary skill in the art to which this disclosure belongs. Thus, as used throughout the instant application, the following terms shall have the following meanings.

“Amino acid” is a molecule having the structure wherein a central carbon atom (the carbon atom) is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino acid carboxylic groups in the dehydration reaction that links one amino acid to another. As a result, when incorporated into a protein, an amino acid is referred to as an “amino acid residue.”

“Protein” or “polypeptide” refers to any polymer of two or more individual amino acids (whether or not naturally occurring) linked via a peptide bond, and occurs when the carboxylcarbon atom of the carboxylic acid group bonded to the carbon of one amino acid (or amino acid residue) becomes covalently bound to the amino nitrogen atom of amino group bonded to the carbon of an adjacent amino acid. The term “protein” is understood to include the terms “polypeptide” and “peptide” (which, at times may be used interchangeably herein) within its meaning. In addition, proteins comprising multiple polypeptide subunits (e.g., DNA polymerase III, RNA polymerase II) or other components (for example, an RNA molecule, as occurs in telomerase) will also be understood to be included within the meaning of “protein” as used herein. Similarly, fragments of proteins and polypeptides are also within the scope of the invention and may be referred to herein as “proteins.” In one aspect of the disclosure, a stabilized protein comprises a chimera of two or more parental peptide segments.

“Peptide segment” refers to a portion or fragment of a larger polypeptide or protein. A peptide segment need not on its own have functional activity, although in some instances, a peptide segment may correspond to a domain of a polypeptide wherein the domain has its own biological activity. A stability-associated peptide segment is a peptide segment found in a polypeptide that promotes stability, function, or folding compared to a related polypeptide lacking the peptide segment. A destabilizing-associated peptide segment is a peptide segment that is identified as causing a loss of stability, function or folding when present in a polypeptide.

A particular amino acid sequence of a given protein (i.e., the polypeptide's “primary structure,” when written from the amino-terminus to carboxy-terminus) is determined by the nucleotide sequence of the coding portion of a mRNA, which is in turn specified by genetic information, typically genomic DNA (including organelle DNA, e.g., mitochondrial or chloroplast DNA). Thus, determining the sequence of a gene assists in predicting the primary sequence of a corresponding polypeptide and more particular the role or activity of the polypeptide or proteins encoded by that gene or polynucleotide sequence.

“Fused,” “operably linked,” and “operably associated” are used interchangeably herein to broadly refer to a chemical or physical coupling of two otherwise distinct domains, wherein each domain has independent biological function. As such, the present disclosure provides heme and reductase domains that are fused to one another such that they function as a holo-enzyme. A fused heme and reductase domain can be connected through peptide linkers such that they are functional or can be fused through other intermediates or chemical bonds. For example, a heme domain and a reductase domain can be part of the same coding sequence, each domain encoded by a heme and reductase polynucleotide, wherein the polynucleotides are in frame such that the polynucleotide when transcribed encodes a single mRNA that when translated comprises both domains (i.e., a heme and reductase domain) as a single polypeptide. Alternatively, both domains can be separately expressed as individual polypeptides and fused to one another using chemical methods. Typically, the coding domains will be linked “in-frame” either directly of separated by a peptide linker and encoded by a single polynucleotide. Various coding sequences for peptide linkers and peptide are known in the art and can include, for example, sequences having identity to the linker sequence separating the domains in the wild-type P450 enzymes comprising SEQ ID NO:1, 2, or 3.

“Polynucleotide” or “nucleic acid sequence” refers to a polymeric form of nucleotides. In some instances a polynucleotide refers to a sequence that is not immediately contiguous with either of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides of the invention can be ribonucleotides, deoxyribonucleotides, or modified forms of either nucleotide. A polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The term polynucleotide encompasses genomic DNA or RNA (depending upon the organism, i.e., RNA genome of viruses), as well as mRNA encoded by the genomic DNA, and cDNA. Polynucleotides encoding P450 from Bacillus megaterium see e.g., GenBank accession no. J04832 and subtilis are known.

“Nucleic acid segment,” “oligonucleotide segment” or “polynucleotide segment” refers to a portion of a larger polynucleotide molecule. The polynucleotide segment need not correspond to an encoded functional domain of a protein; however, in some instances the segment will encode a functional domain of a protein. A polynucleotide segment can be about 6 nucleotides or more in length (e.g., 6-20, 20-50, 50-100, 100-200, 200-300, 300-400 or more nucleotides in length). A stability-associated peptide segment can be encoded by a stability-associated polynucleotide segment, wherein the peptide segment promotes stability, function, or folding compared to a polypeptide lacking the peptide segment.

“Chimera” refers to a combination of at least two segments of at least two different parent proteins. As appreciated by one of skill in the art, the segments need not actually come from each of the parents, as it is the particular sequence that is relevant, and not the physical nucleic acids themselves. For example, a chimeric P450 will have at least two segments from two different parent P450s. The two segments are connected so as to result in a new P450. In other words, a protein will not be a chimera if it has the identical sequence of either one of the parents. A chimeric protein can comprise more than two segments from two different parent proteins. For example, there may be 2, 3, 4, 5-10, 10-20, or more parents for each final chimera or library of chimeras. The segment of each parent enzyme can be very short or very long, the segments can range in length of contiguous amino acids from 1 to the entire length of the protein. In one embodiment, the minimum length is 10 amino acids. In one embodiment, a single crossover point is defined for two parents. The crossover location defines where one parent's amino acid segment will stop and where the next parent's amino acid segment will start. Thus, a simple chimera would only have one crossover location where the segment before that crossover location would belong to one parent and the segment after that crossover location would belong to the second parent. In one embodiment, the chimera has more than one crossover location. For example, 2, 3, 4, 5, 6, 7, 8, 9, 10, 11-30, or more crossover locations. How these crossover locations are named and defined are both discussed below. In an embodiment where there are two crossover locations and two parents, there will be a first contiguous segment from a first parent, followed by a second contiguous segment from a second parent, followed by a third contiguous segment from the first parent. Contiguous is meant to denote that there is nothing of significance interrupting the segments. These contiguous segments are connected to form a contiguous amino acid sequence. For example, a P450 chimera from CYP102A1 (hereinafter “A1”) and CYP102A2 (hereinafter “A2”), with two crossovers at 100 and 150, could have the first 100 amino acids from A1, followed by the next 50 from A2, followed by the remainder of the amino acids from A1, all connected in one contiguous amino acid chain. Alternatively, the P450 chimera could have the first 100 amino acids from A2, the next 50 from A1 and the remainder followed by A2. As appreciated by one of skill in the art, variants of chimeras exist as well as the exact sequences. Thus, not 100% of each segment need be present in the final chimera if it is a variant chimera. The amount that may be altered, either through additional residues or removal or alteration of residues will be defined as the term variant is defined. Of course, as understood by one of skill in the art, the above discussion applies not only to amino acids but also nucleic acids which encode for the amino acids.

“Conservative amino acid substitution” refers to the interchangeability of residues having similar side chains, and thus typically involves substitution of the amino acid in the polypeptide with amino acids within the same or similar defined class of amino acids. By way of example and not limitation, an amino acid with an aliphatic side chain may be substituted with another aliphatic amino acid, e.g., alanine, valine, leucine, isoleucine, and methionine; an amino acid with hydroxyl side chain is substituted with another amino acid with a hydroxyl side chain, e.g., serine and threonine; an amino acids having aromatic side chains is substituted with another amino acid having an aromatic side chain, e.g., phenylalanine, tyrosine, tryptophan, and histidine; an amino acid with a basic side chain is substituted with another amino acid with a basis side chain, e.g., lysine, arginine, and histidine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Isolated polypeptide” refers to a polypeptide which is separated from other contaminants that naturally accompany it, e.g., protein, lipids, and polynucleotides. The term embraces polypeptides which have been removed or purified from their naturally-occurring environment or expression system (e.g., host cell or in vitro synthesis).

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure polypeptide composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species.

“Reference sequence” refers to a defined sequence used as a basis for a sequence comparison. A reference sequence may be a subset of a larger sequence, for example, a segment of a full-length gene or polypeptide sequence. Generally, a reference sequence can be at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptides are typically performed by comparing sequences of the two polynucleotides or polypeptides over a “comparison window” to identify and compare local regions of sequence similarity.

“Sequence identity” means that two amino acid sequences are substantially identical (i.e., on an amino acid-by-amino acid basis) over a window of comparison. The term “sequence similarity” refers to similar amino acids that share the same biophysical characteristics. The term “percentage of sequence identity” or “percentage of sequence similarity” is calculated by comparing two optimally aligned sequences over the window of comparison, determining the number of positions at which the identical residues (or similar residues) occur in both polypeptide sequences to yield the number of matched positions, dividing the number of matched positions by the total number of positions in the window of comparison (i.e., the window size), and multiplying the result by 100 to yield the percentage of sequence identity (or percentage of sequence similarity). With regard to polynucleotide sequences, the terms sequence identity and sequence similarity have comparable meaning as described for protein sequences, with the term “percentage of sequence identity” indicating that two polynucleotide sequences are identical (on a nucleotide-by-nucleotide basis) over a window of comparison. As such, a percentage of polynucleotide sequence identity (or percentage of polynucleotide sequence similarity, e.g., for silent substitutions or other substitutions, based upon the analysis algorithm) also can be calculated. Maximum correspondence can be determined by using one of the sequence algorithms described herein (or other algorithms available to those of ordinary skill in the art) or by visual inspection.

As applied to polypeptides, the term substantial identity or substantial similarity means that two peptide sequences, when optimally aligned, such as by the programs BLAST, GAP or BESTFIT using default gap weights or by visual inspection, share sequence identity or sequence similarity. Similarly, as applied in the context of two nucleic acids, the term substantial identity or substantial similarity means that the two nucleic acid sequences, when optimally aligned, such as by the programs BLAST, GAP or BESTFIT using default gap weights (described in detail below) or by visual inspection, share sequence identity or sequence similarity.

One example of an algorithm that is suitable for determining percent sequence identity or sequence similarity is the FASTA algorithm, which is described in Pearson, W. R. & Lipman, D. J., (1988) Proc. Natl. Acad. Sci. USA 85:2444. See also, W. R. Pearson, (1996) Methods Enzymology 266:227-258. Preferred parameters used in a FASTA alignment of DNA sequences to calculate percent identity or percent similarity are optimized, BL50 Matrix 15: −5, k-tuple=2; joining penalty=40, optimization=28; gap penalty −12, gap length penalty=−2; and width=16.

Another example of a useful algorithm is PILEUP. PILEUP creates a multiple sequence alignment from a group of related sequences using progressive, pairwise alignments to show relationship and percent sequence identity or percent sequence similarity. It also plots a tree or dendogram showing the clustering relationships used to create the alignment. PILEUP uses a simplification of the progressive alignment method of Feng & Doolittle, (1987) J. Mol. Evol. 35:351-360. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity (or percent sequence similarity) relationship using the following parameters: default gap weight (3.00), default gap length weight (0.10), and weighted end gaps. PILEUP can be obtained from the GCG sequence analysis software package, e.g., version 7.0 (Devereaux et al., (1984) Nuc. Acids Res. 12:387-395).

Another example of an algorithm that is suitable for multiple DNA and amino acid sequence alignments is the CLUSTALW program (Thompson, J. D. et al., (1994) Nuc. Acids Res. 22:4673-4680). CLUSTALW performs multiple pairwise comparisons between groups of sequences and assembles them into a multiple alignment based on sequence identity. Gap open and Gap extension penalties were 10 and 0.05 respectively. For amino acid alignments, the BLOSUM algorithm can be used as a protein weight matrix (Henikoff and Henikoff, (1992) Proc. Natl. Acad. Sci. USA 89:10915-10919).

“Functional” refers to a polypeptide which possesses either the native biological activity of the naturally-produced proteins of its type, or any specific desired activity, for example as judged by its ability to bind to ligand molecules or carry out an enzymatic reaction.

“Heme domain” refers to an amino acid sequence capable of binding an iron-complexing structure, such as porphyrin. Generally, iron is complexed in a porphyrin ring, which may differ in side chain. For example, in Bacillus megatarium cytochrome p450 BM3, the porphyrin is typically protoporphyrin IX.

“Reductase domain” refers to an amino acid sequence capable of binding a flavin molecule, such as flavin adenine dinucleotide (FAD) and/or flavin adenine mononucleotide (FMN). Generally, these forms of flavin are present as a prosthetic group in the reductase domain and functions in electron transfer reactions. The domain structure of the cytochrome p450 BMS enzyme is described in Govindarag and Poulos, (1996) J. Biol. Chem 272(12):7915-7921, incorporated herein by reference.

“Isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis).

The present disclosure describes a directed SCHEMA recombination library to generate cytochrome p450 enzymes based on a particularly well-studied member of this diverse enzyme family, cytochrome P450 BM3 (CYP102A1, or “A1”; SEQ ID NO:1; see also GenBank Accession No. J04832, which is incorporated herein by reference) from Bacillus megaterium. SCHEMA is a computational based method for predicting which fragments of homologous proteins can be recombined without affecting the structural integrity of the protein (see, e.g., Meyer et al., (2003) Protein Sci., 12:1686-1693). This computational approached identified seven recombination points in the heme domain of the cytochrome p450 enzyme, thereby allowing the formation of a library of heme domain polypeptides, where each polypeptide comprise eight segments. Segments were based on three naturally occurring cytochrome p450 variants, CYP102A1, CYP102A2, and CYP102A3. Chimeras with higher stability are identifiable by determining the additive contribution of each segment to the overall stability, either by use of linear regression of sequence-stability data, or by reliance on consensus analysis of the MSAs of folded versus unfolded proteins. SCHEMA recombination ensures that the chimeras retain biological function and exhibit high sequence diversity by conserving important functional residues while exchanging tolerant ones.

As presented in this disclosure, it has been found that when these recombined, functional cytochrome p450 heme domains enzyme are fused to the reductase domain to generate functional monooxygenase activity, the enzymes have different substrate activity profiles as well as changes in enzyme properties, such as enzyme activity, as compared to a unrecombined heme domain fused to a reductase domain or as compared to the parent cytochrome p450 enzyme. Because of differences in activity profiles, these engineered cytochrome p450 holoenzymes provide a unique basis to screen for activities on novel substrates, including drug compounds, as well as identifying activity against organic chemicals, such as environmental toxins, not normally recognized by the parent enzymes.

Thus, as illustrated by various embodiments herein, the disclosure provides heme-reductase polypeptides, wherein the reductase domain is operably linked or fused to the heme domain (see, e.g., Table 8 for exemplary sequences of segments and reductase domains). In some embodiments, the polypeptide comprises a chimeric heme domain and a reductase domain; the heme domain comprising from N- to C-terminus: (segment 1)-(segment 2)-(segment 3)-(segment 4)-(segment 5)-(segment 6)-(segment 7)-(segment 8);

wherein segment 1 is amino acid residue from about 1 to about x₁ of SEQ ID NO:1 (“1”), SEQ ID NO:2 (“2”) or SEQ ID NO:3 (“3”); segment 2 is from about amino acid residue x₁ to about x₂ of SEQ ID NO:1 (“1”), SEQ ID NO:2 (“2”) or SEQ ID NO:3 (“3”); segment 3 is from about amino acid residue x₂ to about x₃ of SEQ ID NO:1 (“1”), SEQ ID NO:2 (“2”) or SEQ ID NO:3 (“3”); segment 4 is from about amino acid residue x₃ to about x₄ of SEQ ID NO:1 (“1”), SEQ ID NO:2 (“2”) or SEQ ID NO:3 (“3”); segment 5 is from about amino acid residue x₄ to about x₅ of SEQ ID NO:1 (“1”), SEQ ID NO:2 (“2”) or SEQ ID NO:3 (“3”); segment 6 is from about amino acid residue x₅ to about x₆ of SEQ ID NO:1 (“1”), SEQ ID NO:2 (“2”) or SEQ ID NO:3 (“3”); segment 7 is from about amino acid residue x₆ to about x₇ of SEQ ID NO:1 (“1”), SEQ ID NO:2 (“2”) or SEQ ID NO:3 (“3”); and segment 8 is from about amino acid residue x₇ to about x₈ of SEQ ID NO:1 (“1”), SEQ ID NO:2 (“2”) or SEQ ID NO:3 (“3”);

wherein: x₁ is residue 62, 63, 64, 65 or 66 of SEQ ID NO:1, or residue 63, 64, 65, 66 or 67 of SEQ ID NO:2 or SEQ ID NO:3; x₂ is residue 120, 121, 122, 123, 124, 125, 126, 127, 128, 129, 130, 132 or 132 of SEQ ID NO:1, or residue 121, 122, 123, 124, 125, 126, 127, 128, 129, 130, 131, 132, or 133 of SEQ ID NO:2 or SEQ ID NO:3; x₃ is residue 164, 165, 166, 167, 168, 169, 170, 171, 172, 173, 174, 175, 176, or 177 of SEQ ID NO:1, or residue 165, 166, 167, 168, 169, 170, 171, 172, 173, 174, 175, 176, 177, or 178 of SEQ ID NO:2 or SEQ ID NO:3; x₄ is residue 214, 215, 216, 217 or 218 of SEQ ID NO:1, or residue 215, 216, 217, 218 or 219 of SEQ ID NO:2 or SEQ ID NO:3; x₅ is residue 266, 267, 268, 269 or 270 of SEQ ID NO:1, or residue 268, 269, 270, 271 or 272 of SEQ ID NO:2 or SEQ ID NO:3; x₆ is residue 326, 327, 328, 329 or 330 of SEQ ID NO:1, or residue 328, 329, 330, 331 or 332 of SEQ ID NO:2 or SEQ ID NO:3; x₇ is residue 402, 403, 404, 405 or 406 of SEQ ID NO:1, or residue 404, 405, 405, 407 or 408 of SEQ ID NO:2 or SEQ ID NO:3; and x₈ is an amino acid residue corresponding to the C-terminus of the heme domain of CYP102A1, CYP102A2 or CYP102A3 or the C-terminus of SEQ ID NO:1, SEQ ID NO:2 or SEQ ID NO:3;

wherein the heme domain has a general (chimeric) structure selected from the group consisting of: 11112212, 11113233, 11113311, 11131313, 11132223, 11132232, 11133231, 11212112, 11212333, 11213133, 11213231, 11232111, 11232232, 11232333, 11311233, 11312233, 11313233, 11313333, 11331312, 11331333, 11332212, 11332233, 11332333, 11333212, 12112333, 12113221, 12211232, 12211333, 12212112, 12212211, 12212212, 12212223, 12212332, 12213212, 12232111, 12232112, 12232232, 12232233, 12232332, 12233112, 12233212, 12313331, 12322333, 12331123, 12331333, 12332223, 12332333, 12333331, 12333333, 13113311, 13213131, 13221231, 13222212, 13233212, 13332333, 13333122, 13333132, 13333211, 13333233, 21111321, 21111323, 21111333, 21112122, 21112123, 21112132, 21112212, 21112222, 21112232, 21112233, 21112311, 21112312, 21112331, 21112332, 21112333, 21113111, 21113112, 21113122, 21113133, 21113211, 21113212, 21113221, 21113223, 21113312, 21113321, 21113322, 21113333, 21131121, 21132112, 21132113, 21132212, 21132222, 21132311, 21132313, 21132321, 21132323, 21133112, 21133113, 21133131, 21133211, 21133222, 21133223, 21133232, 21133233, 21133312, 21133313, 21133321, 21133322, 21133331, 21133332, 21211223, 21211321, 21212111, 21212112, 21212122, 21212123, 21212133, 21212212, 21212213, 21212231, 21212233, 21212321, 21212332, 21212333, 21213121, 21213212, 21213223, 21213231, 21213321, 21213332, 21222112, 21231232, 21231233, 21232112, 21232122, 21232132, 21232212, 21232222, 21232231, 21232232, 21232233, 21232321, 21232322, 21232323, 21232332, 21233111, 21233132, 21233212, 21233221, 21233233, 21233312, 21233321, 21311122, 21311223, 21311231, 21311233, 21311311, 21311313, 21311331, 21311333, 21312111, 21312112, 21312122, 21312123, 21312133, 21312211, 21312213, 21312222, 21312223, 21312231, 21312233, 21312311, 21312313, 21312321, 21312322, 21312323, 21312331, 21312332, 21312333, 21313111, 21313112, 21313122, 21313221, 21313231, 21313233, 21313311, 21313312, 21313313, 21313322, 21313331, 21313333, 21331223, 21331332, 21331333, 21332111, 21332112, 21332113, 21332122, 21332131, 21332212, 21332221, 21332223, 21332231, 21332233, 21332312, 21332322, 21332323, 21332331, 21332332, 21332333, 21333111, 21333122, 21333131, 21333132, 21333211, 21333212, 21333221, 21333223, 21333233, 21333312, 21333321, 22313333, 21333333, 22111223, 22111332, 22112111, 22112131, 22112211, 22112223, 22112233, 22112321, 22112323, 22112331, 22112333, 22113111, 22113211, 22113223, 22113232, 22113233, 22113313, 22113323, 22113332, 22131221, 22132112, 22132113, 22132212, 22132231, 22132233, 22132312, 22132323, 22132331, 22133112, 22133211, 22133212, 22133232, 22133312, 22133322, 22133323, 22212111, 22212123, 22212131, 22212212, 22212232, 22212312, 22212321, 22212322, 22212333, 22213111, 22213112, 22213132, 22213212, 22213222, 22213223, 22213312, 22213321, 22222121, 22231221, 22231223, 22231312, 22231322, 22232111, 22232112, 22232121, 22232122, 22232123, 22232212, 22232222, 22232223, 22232232, 22232233, 22232311, 22232312, 22232322, 22232323, 22232331, 22232333, 22233112, 22233211, 22233212, 22233221, 22233222, 22233223, 22233312, 22233323, 22233332, 22311123, 22311212, 22311231, 22311233, 22311331, 22311333, 22312111, 22312123, 22312132, 22312133, 22312211, 22312221, 22312222, 22312223, 22312231, 22312232, 22312233, 22312311, 22312312, 22312322, 22312331, 22312332, 22312333, 22313122, 22313212, 22313221, 22313222, 22313231, 22313232, 22313233, 22313323, 22313331, 22313332, 22323313, 22331123, 22331133, 22331221, 22331223, 22331323, 22331332, 22332112, 22332113, 22332121, 22332123, 22332132, 22332211, 22332221, 22332222, 22332223, 22332232, 22332233, 22332312, 22332321, 22332322, 22332332, 22333112, 22333122, 22333131, 22333132, 22333133, 22333211, 22333212, 22333221, 22333222, 22333223, 22333231, 22333311, 22333313, 22333321, 22333323, 22333332, 23112213, 23112221, 23112223, 23112233, 23112323, 23112333, 23113111, 23113112, 23113121, 23113131, 23113212, 23113311, 23113312, 23113323, 23113332, 23122212, 23131323, 23132111, 23132121, 23132212, 23132221, 23132232, 23132233, 23132311, 23132322, 23132323, 23133112, 23133113, 23133121, 23133233, 23133311, 23133321, 23133331, 23133333, 23211132, 23212112, 23212211, 23212212, 23212221, 23212222, 23212231, 23212332, 23212333, 23213112, 23213121, 23213123, 23213211, 23213212, 23213223, 23213232, 23213311, 23213322, 23213333, 23231233, 23232113, 23232131, 23232211, 23232212, 23232311, 23232323, 23233212, 23233221, 23233231, 23233232, 23233312, 23233333, 23311233, 23311323, 23312112, 23312121, 23312122, 23312123, 23312131, 23312223, 23312311, 23312312, 23312323, 23313111, 23313133, 23313212, 23313222, 23313232, 23313233, 23313323, 23313333, 23331233, 23331323, 23332112, 23332221, 23332222, 23332223, 23332231, 23332311, 23332323, 23332331, 23333111, 23333123, 23333131, 23333211, 23333212, 23333213, 23333222, 23333223, 23333232, 23333233, 23333311, 23333312, 23333323, 31111233, 31112231, 31112333, 31113131, 31113132, 31113222, 31113323, 31113331, 31113332, 31131233, 31132231, 31132232, 31132333, 31133233, 31133331, 31211131, 31211232, 31212112, 31212212, 31212232, 31212321, 31212323, 31212331, 31212332, 31212333, 31213232, 31213233, 31213323, 31213331, 31213332, 31232231, 31232312, 31232333, 31233221, 31233222, 31233233, 31311231, 31311233, 31311332, 31312113, 31312133, 31312212, 31312222, 31312231, 31312233, 31312323, 31312332, 31312333, 31313111, 31313131, 31313132, 31313133, 31313223, 31313232, 31313233, 31313333, 31331331, 31331333, 31332131, 31332133, 31332232, 31332233, 31332312, 31332322, 31332323, 31332333, 31333233, 31333322, 31333332, 31333333, 32111333, 32112212, 32112313, 32112321, 32113131, 32113232, 32113233, 32131133, 32132232, 32132233, 32132331, 32133111, 32133232, 32133233, 32133331, 32211323, 32212133, 32212231, 32212232, 32212233, 32212321, 32212323, 32212332, 32212333, 32213123, 32213132, 32213231, 32213333, 32232131, 32232322, 32232331, 32232333, 32233222, 32233332, 32311131, 32311323, 32312212, 32312231, 32312233, 32312311, 32312322, 32312323, 32312331, 32312332, 32312333, 32313133, 32313231, 32313232, 32313233, 32313313, 32313332, 32313333, 32332133, 32332223, 32332231, 32332232, 32332322, 32332323, 32332331, 32332332, 32332333, 32333223, 32333232, 32333233, 32333312, 32333323, 32333333, 33113111, 33113211, 33113212, 33113233, 33131333, 33133131, 33133333, 33212213, 33212311, 33212333, 33213211, 33213232, 33213333, 33232233, 33232312, 33232333, 33233131, 33233233, 33233333, 33311231, 33312133, 33312322, 33312333, 33313223, 33313233, 33313323, 33313333, 33331232, 33331233, 33331333, 33332131, 33332133, 33332221, 33332232, 33332233, 33332323, 33332333, 33333123, 33333231, 33333232, 33333233, 33333321, and 33333323,

wherein the reductase domain comprises at least 50% identity to the reductase domain of SEQ ID NO:1, 2 or 3, and wherein the polypeptide has monooxygenase activity.

In some embodiments, the heme domain of the heme-reductase polypeptide has a chimeric segment structure selected from the group consisting of:

21112233, 21112331, 21112333, 21113333, 21212233, 21212333, 21311231, 21311233, 21311311, 21311313, 21311331, 21311333, 21312133, 21312211, 21312213, 21312231, 21312311, 21312313, 21312331, 21312332, 21312333, 21313231, 21313233, 21313313, 21313331, 21313333, 22112233, 22112333, 22212333, 22311233, 22311331, 22311333, 22312231, 22312233, 22312331, 22312333, 22313231, 22313233, 22313331, and 22313333.

In some embodiments, specifically excluded from selection and use are heme domains having a chimeric segment structure selected from the group consisting of:

11113311, 12112333, 21113312, 21313111, 21313311, 21333233, 22132231, 22213132, 22312333, 22313233, 23132233, 32312231, 32312333, and 32313233.

In various embodiments, the heme domain individually or as a holoenzyme (i.e., linked to a reductase domain) can have a CO-binding peak at 450 nm.

In some embodiments, the polypeptide has improved monooxygenase activity compared to a wild-type polypeptide of SEQ ID NO:1, 2, or 3. The activity of the polypeptide can be measured with any one or combination of substrates as described in the examples, including, among others, diphenyl ether, ethoxybenzene, ethylphenoxyacetate, 3 phenoxytoluene, 2-phenoxyethanol, ethyl-4-phenylbutyrate, zoxazolamine, chorzoxazone, propranolol, and tolbutamide. As will be apparent to the skilled artisan, other compounds within the class of compounds exemplified by those discussed in the examples can be tested and used. An exemplary substrate for purposes of comparison between enzymes is 2-phenoxyethanol using the reaction conditions as described in the examples.

In some embodiments, the reductase domain of the polypeptides can comprise an amino acid sequence that has at least 60%, 70%, 80%, 90%, 95%, 96%, 97%, 98%, or 99% or more identity as compared to the reference reductase domain of SEQ ID NO:1, SEQ ID NO:2, or SEQ ID NO:3, wherein the reductase domain is functional when fused to the chimeric heme domain.

In some embodiments, the reductase domain of the polypeptide comprises the reductase domain of SEQ ID NO:1.

In some embodiments, the reductase domain of the polypeptide comprises the reductase domain of SEQ ID NO:2.

In some embodiments, the reductase domain of the polypeptide comprises the reductase domain of SEQ ID NO:3.

In various embodiments, the substrate specificity of the polypeptide is different when compared to the wild-type polypeptide of SEQ ID NO:1, 2, or 3, and can be measured using any one or combination of substrates as described in the examples.

In some embodiments, the polypeptide can be have various changes to the amino acid sequence with respect to a reference sequence. The changes can be a substitution, deletion, or insertion of one or more amino acids. Where the change is a substitution, the change can be a conservative, a non-conservative substitution, or a combination of conservative and non-conservative substitutions.

Thus, in some embodiments, the polypeptides can comprise a general structure from N-terminus to C-terminus:

(segment 1)-(segment 2)-(segment 3)-(segment 4)-(segment 5)(segment 6)-(segment 7)-(segment 8)-reductase domain,

wherein segment 1 comprises an amino acid sequence from about residue 1 to about x₁ of SEQ ID NO:1 (“1”), SEQ ID NO:2 (“2”) or SEQ ID NO:3 (“3”) and having about 1-10 conservative amino acid substitutions; segment 2 is from about amino acid residue x₁ to about x₂ of SEQ ID NO:1 (“1”), SEQ ID NO:2 (“2”) or SEQ ID NO:3 (“3”) and having about 1-10 conservative amino acid substitutions; segment 3 is from about amino acid residue x₂ to about x₃ of SEQ ID NO:1 (“1”), SEQ ID NO:2 (“2”) or SEQ ID NO:3 (“3”) and having about 1-10 conservative amino acid substitutions; segment 4 is from about amino acid residue x₃ to about x₄ of SEQ ID NO:1 (“1”), SEQ ID NO:2 (“2”) or SEQ ID NO:3 (“3”) and having about 1-10 conservative amino acid substitutions; segment 5 is from about amino acid residue x₄ to about x₅ of SEQ ID NO:1 (“1”), SEQ ID NO:2 (“2”) or SEQ ID NO:3 (“3”) and having about 1-10 conservative amino acid substitutions; segment 6 is from about amino acid residue x₅ to about x₆ of SEQ ID NO:1 (“1”), SEQ ID NO:2 (“2”) or SEQ ID NO:3 (“3”) and having about 1-10 conservative amino acid substitutions; segment 7 is from about amino acid residue x₆ to about x₇ of SEQ ID NO:1 (“1”), SEQ ID NO:2 (“2”) or SEQ ID NO:3 (“3”) and having about 1-10 conservative amino acid substitutions; and segment 8 is from about amino acid residue x₇ to about x₈ of SEQ ID NO:1 (“1”), SEQ ID NO:2 (“2”) or SEQ ID NO:3 (“3”) and having about 1-10 conservative amino acid substitutions;

wherein x₁ is residue 62, 63, 64, 65 or 66 of SEQ ID NO:1, or residue 63, 64, 65, 66 or 67 of SEQ ID NO:2 or SEQ ID NO:3; x₂ is residue 120, 121, 122, 123, 124, 125, 126, 127, 128, 129, 130, 132 or 132 of SEQ ID NO:1, or residue 121, 122, 123, 124, 125, 126, 127, 128, 129, 130, 131, 132, or 133 of SEQ ID NO:2 or SEQ ID NO:3; x₃ is residue 164, 165, 166, 167, 168, 169, 170, 171, 172, 173, 174, 175, 176, or 177 of SEQ ID NO:1, or residue 165, 166, 167, 168, 169, 170, 171, 172, 173, 174, 175, 176, 177, or 178 of SEQ ID NO:2 or SEQ ID NO:3; x₄ is residue 214, 215, 216, 217 or 218 of SEQ ID NO:1, or residue 215, 216, 217, 218 or 219 of SEQ ID NO:2 or SEQ ID NO:3; x₅ is residue 266, 267, 268, 269 or 270 of SEQ ID NO:1, or residue 268, 269, 270, 271 or 272 of SEQ ID NO:2 or SEQ ID NO:3; x₆ is residue 326, 327, 328, 329 or 330 of SEQ ID NO:1, or residue 328, 329, 330, 331 or 332 of SEQ ID NO:2 or SEQ ID NO:3; x₇ is residue 402, 403, 404, 405 or 406 of SEQ ID NO:1, or residue 404, 405, 405, 407 or 408 of SEQ ID NO:2 or SEQ ID NO:3; and x₈ is an amino acid residue corresponding to the C-terminus of the heme domain of CYP102A1, CYP102A2 or CYP102A3 or the C-terminus of SEQ ID NO:1, SEQ ID NO:2 or SEQ ID NO:3;

wherein the heme domain has a general (chimeric) structure selected from the group consisting of:

11112212, 11113233, 11113311, 11131313, 11132223, 11132232, 11133231, 11212112, 11212333, 11213133, 11213231, 11232111, 11232232, 11232333, 11311233, 11312233, 11313233, 11313333, 11331312, 11331333, 11332212, 11332233, 11332333, 11333212, 12112333, 12113221, 12211232, 12211333, 12212112, 12212211, 12212212, 12212223, 12212332, 12213212, 12232111, 12232112, 12232232, 12232233, 12232332, 12233112, 12233212, 12313331, 12322333, 12331123, 12331333, 12332223, 12332333, 12333331, 12333333, 13113311, 13213131, 13221231, 13222212, 13233212, 13332333, 13333122, 13333132, 13333211, 13333233, 21111321, 21111323, 21111333, 21112122, 21112123, 21112132, 21112212, 21112222, 21112232, 21112233, 21112311, 21112312, 21112331, 21112332, 21112333, 21113111, 21113112, 21113122, 21113133, 21113211, 21113212, 21113221, 21113223, 21113312, 21113321, 21113322, 21113333, 21131121, 21132112, 21132113, 21132212, 21132222, 21132311, 21132313, 21132321, 21132323, 21133112, 21133113, 21133131, 21133211, 21133222, 21133223, 21133232, 21133233, 21133312, 21133313, 21133321, 21133322, 21133331, 21133332, 21211223, 21211321, 21212111, 21212112, 21212122, 21212123, 21212133, 21212212, 21212213, 21212231, 21212233, 21212321, 21212332, 21212333, 21213121, 21213212, 21213223, 21213231, 21213321, 21213332, 21222112, 21231232, 21231233, 21232112, 21232122, 21232132, 21232212, 21232222, 21232231, 21232232, 21232233, 21232321, 21232322, 21232323, 21232332, 21233111, 21233132, 21233212, 21233221, 21233233, 21233312, 21233321, 21311122, 21311223, 21311231, 21311233, 21311311, 21311313, 21311331, 21311333, 21312111, 21312112, 21312122, 21312123, 21312133, 21312211, 21312213, 21312222, 21312223, 21312231, 21312233, 21312311, 21312313, 21312321, 21312322, 21312323, 21312331, 21312332, 21312333, 21313111, 21313112, 21313122, 21313221, 21313231, 21313233, 21313311, 21313312, 21313313, 21313322, 21313331, 21313333, 21331223, 21331332, 21331333, 21332111, 21332112, 21332113, 21332122, 21332131, 21332212, 21332221, 21332223, 21332231, 21332233, 21332312, 21332322, 21332323, 21332331, 21332332, 21332333, 21333111, 21333122, 21333131, 21333132, 21333211, 21333212, 21333221, 21333223, 21333233, 21333312, 21333321, 22313333, 21333333, 22111223, 22111332, 22112111, 22112131, 22112211, 22112223, 22112233, 22112321, 22112323, 22112331, 22112333, 22113111, 22113211, 22113223, 22113232, 22113233, 22113313, 22113323, 22113332, 22131221, 22132112, 22132113, 22132212, 22132231, 22132233, 22132312, 22132323, 22132331, 22133112, 22133211, 22133212, 22133232, 22133312, 22133322, 22133323, 22212111, 22212123, 22212131, 22212212, 22212232, 22212312, 22212321, 22212322, 22212333, 22213111, 22213112, 22213132, 22213212, 22213222, 22213223, 22213312, 22213321, 22222121, 22231221, 22231223, 22231312, 22231322, 22232111, 22232112, 22232121, 22232122, 22232123, 22232212, 22232222, 22232223, 22232232, 22232233, 22232311, 22232312, 22232322, 22232323, 22232331, 22232333, 22233112, 22233211, 22233212, 22233221, 22233222, 22233223, 22233312, 22233323, 22233332, 22311123, 22311212, 22311231, 22311233, 22311331, 22311333, 22312111, 22312123, 22312132, 22312133, 22312211, 22312221, 22312222, 22312223, 22312231, 22312232, 22312233, 22312311, 22312312, 22312322, 22312331, 22312332, 22312333, 22313122, 22313212, 22313221, 22313222, 22313231, 22313232, 22313233, 22313323, 22313331, 22313332, 22323313, 22331123, 22331133, 22331221, 22331223, 22331323, 22331332, 22332112, 22332113, 22332121, 22332123, 22332132, 22332211, 22332221, 22332222, 22332223, 22332232, 22332233, 22332312, 22332321, 22332322, 22332332, 22333112, 22333122, 22333131, 22333132, 22333133, 22333211, 22333212, 22333221, 22333222, 22333223, 22333231, 22333311, 22333313, 22333321, 22333323, 22333332, 23112213, 23112221, 23112223, 23112233, 23112323, 23112333, 23113111, 23113112, 23113121, 23113131, 23113212, 23113311, 23113312, 23113323, 23113332, 23122212, 23131323, 23132111, 23132121, 23132212, 23132221, 23132232, 23132233, 23132311, 23132322, 23132323, 23133112, 23133113, 23133121, 23133233, 23133311, 23133321, 23133331, 23133333, 23211132, 23212112, 23212211, 23212212, 23212221, 23212222, 23212231, 23212332, 23212333, 23213112, 23213121, 23213123, 23213211, 23213212, 23213223, 23213232, 23213311, 23213322, 23213333, 23231233, 23232113, 23232131, 23232211, 23232212, 23232311, 23232323, 23233212, 23233221, 23233231, 23233232, 23233312, 23233333, 23311233, 23311323, 23312112, 23312121, 23312122, 23312123, 23312131, 23312223, 23312311, 23312312, 23312323, 23313111, 23313133, 23313212, 23313222, 23313232, 23313233, 23313323, 23313333, 23331233, 23331323, 23332112, 23332221, 23332222, 23332223, 23332231, 23332311, 23332323, 23332331, 23333111, 23333123, 23333131, 23333211, 23333212, 23333213, 23333222, 23333223, 23333232, 23333233, 23333311, 23333312, 23333323, 31111233, 31112231, 31112333, 31113131, 31113132, 31113222, 31113323, 31113331, 31113332, 31131233, 31132231, 31132232, 31132333, 31133233, 31133331, 31211131, 31211232, 31212112, 31212212, 31212232, 31212321, 31212323, 31212331, 31212332, 31212333, 31213232, 31213233, 31213323, 31213331, 31213332, 31232231, 31232312, 31232333, 31233221, 31233222, 31233233, 31311231, 31311233, 31311332, 31312113, 31312133, 31312212, 31312222, 31312231, 31312233, 31312323, 31312332, 31312333, 31313111, 31313131, 31313132, 31313133, 31313223, 31313232, 31313233, 31313333, 31331331, 31331333, 31332131, 31332133, 31332232, 31332233, 31332312, 31332322, 31332323, 31332333, 31333233, 31333322, 31333332, 31333333, 32111333, 32112212, 32112313, 32112321, 32113131, 32113232, 32113233, 32131133, 32132232, 32132233, 32132331, 32133111, 32133232, 32133233, 32133331, 32211323, 32212133, 32212231, 32212232, 32212233, 32212321, 32212323, 32212332, 32212333, 32213123, 32213132, 32213231, 32213333, 32232131, 32232322, 32232331, 32232333, 32233222, 32233332, 32311131, 32311323, 32312212, 32312231, 32312233, 32312311, 32312322, 32312323, 32312331, 32312332, 32312333, 32313133, 32313231, 32313232, 32313233, 32313313, 32313332, 32313333, 32332133, 32332223, 32332231, 32332232, 32332322, 32332323, 32332331, 32332332, 32332333, 32333223, 32333232, 32333233, 32333312, 32333323, 32333333, 33113111, 33113211, 33113212, 33113233, 33131333, 33133131, 33133333, 33212213, 33212311, 33212333, 33213211, 33213232, 33213333, 33232233, 33232312, 33232333, 33233131, 33233233, 33233333, 33311231, 33312133, 33312322, 33312333, 33313223, 33313233, 33313323, 33313333, 33331232, 33331233, 33331333, 33332131, 33332133, 33332221, 33332232, 33332233, 33332323, 33332333, 33333123, 33333231, 33333232, 33333233, 33333321, and 33333323,

wherein the reductase domain comprises at least 50% identity to the reductase domain of SEQ ID NO:1, 2 or 3, and wherein the polypeptide has monooxygenase activity.

In some embodiments, the heme domain for the substitution mutations is selected from the group consisting of:

21112233, 21112331, 21112333, 21113333, 21212233, 21212333, 21311231, 21311233, 21311311, 21311313, 21311331, 21311333, 21312133, 21312211, 21312213, 21312231, 21312311, 21312313, 21312331, 21312332, 21312333, 21313231, 21313233, 21313313, 21313331, 21313333, 22112233, 22112333, 22212333, 22311233, 22311331, 22311333, 22312231, 22312233, 22312331, 22312333, 22313231, 22313233, 22313331, and 22313333.

As above, the heme domain in these mutated variants, individually or as a holoenzyme (i.e., linked to a reductase domain), can have a CO-binding peak at 450 nm.

In some embodiments, the number of substitutions can be 2, 3, 4, 5, 6, 8, 9, or 10, or more amino acid substitutions. In some embodiments, the amino acid residues for substitution are selected from those described below.

In some embodiments, the conservative amino acid substitutions exclude substitutions at residues: (a) 47, 78, 82, 94, 142, 175, 184, 205, 226, 236, 252, 255, 290, 328, and 353 of SEQ ID NO:1; and (b) 48, 79, 83, 95, 143, 176, 185, 206, 227, 238, 254, 257, 292, 330, and 355 of SEQ ID NO:2 or SEQ ID NO:3.

In some embodiments, the polypeptide comprises (1) a Z1 amino acid residue at positions: (a) 47, 82, 142, 205, 236, 252, and 255 of SEQ ID NO:1; (b) 48, 83, 143, 206, 238, 254, and 257 of SEQ ID NO:2 or SEQ ID NO:3; (2) a Z2 amino acid residue at positions: (a) 94, 175, 184, 290, and 353 of SEQ ID NO:1; (b) 95, 176, 185, 292, and 355 of SEQ ID NO:2 or SEQ ID NO:3; (3) a Z3 amino acid residue at position: (a) 226 of SEQ ID NO:1; (b) 227 of SEQ ID NO:2 or SEQ ID NO:3; and (4) a Z4 amino acid residue at positions: (a) 78 and 328 of SEQ ID NO:1; (b) 79 and 330 of SEQ ID NO:2 or SEQ ID NO:3, wherein a Z1 amino acid residue includes glycine (G), asparagine (N), glutamine (Q), serine (S), threonine (T), tyrosine (Y), or cysteine (C). A Z2 amino acid residue includes alanine (A), valine (V), leucine (L), isoleucine (I), proline (P), or methionine (M). A Z3 amino acid residue includes lysine (K), or arginine (R). A Z4 amino acid residue includes tyrosine (Y), phenylalanine (F), tryptophan (W), or histidine (H).

In some embodiments, the functional cytochrome p450 polypeptides can have monooxygenase activity, such as for a defined substrate discussed in the Examples, and also have a level of amino acid sequence identity to a reference cytochrome p450 enzyme, or segments thereof. The reference enzyme or segment, can be that of a wild-type (e.g., naturally occurring) or an engineered enzyme. Thus, in some embodiments, the polypeptides of the disclosure can comprise a general structure from N-terminus to C-terminus:

(segment 1)-(segment 2)-(segment 3)-(segment 4)-(segment 5)(segment 6)-(segment 7)-(segment 8)-reductase domain, wherein segment 1 comprises at least 50-100% identity to the sequence of SEQ ID NO:4, 5, or 6; wherein segment 2 comprises at least 50-100% identity to the sequence of SEQ ID NO:7, 8, or 9; wherein segment 3 comprises at least 50-100% identity to the sequence of SEQ ID NO:10, 11 or 12; segment 4 comprises at least 50-100% identity to the sequence of SEQ ID NO:13, 14, or 15; segment 5 comprises at least 50-100% identity to the sequence of SEQ ID NO:16, 17, or 18; segment 6 comprises at least 50-100% identity to the sequence of SEQ ID NO:19, 20, or 21; segment 7 comprises at least 50-100% identity to the sequence of SEQ ID NO:22, 23, or 24; and segment 8 comprises at least 50-100% identity to a sequence of SEQ ID NO:25, 26, or 27,

wherein the reductase domain comprises at least 50-100% identity to SEQ ID NO:35, and wherein the polypeptide has monooxygenase activity.

As noted above, the reference chimeric heme domain can be a chimeric structure selected from:

11112212, 11113233, 11113311, 11131313, 11132223, 11132232, 11133231, 11212112, 11212333, 11213133, 11213231, 11232111, 11232232, 11232333, 11311233, 11312233, 11313233, 11313333, 11331312, 11331333, 11332212, 11332233, 11332333, 11333212, 12112333, 12113221, 12211232, 12211333, 12212112, 12212211, 12212212, 12212223, 12212332, 12213212, 12232111, 12232112, 12232232, 12232233, 12232332, 12233112, 12233212, 12313331, 12322333, 12331123, 12331333, 12332223, 12332333, 12333331, 12333333, 13113311, 13213131, 13221231, 13222212, 13233212, 13332333, 13333122, 13333132, 13333211, 13333233, 21111321, 21111323, 21111333, 21112122, 21112123, 21112132, 21112212, 21112222, 21112232, 21112233, 21112311, 21112312, 21112331, 21112332, 21112333, 21113111, 21113112, 21113122, 21113133, 21113211, 21113212, 21113221, 21113223, 21113312, 21113321, 21113322, 21113333, 21131121, 21132112, 21132113, 21132212, 21132222, 21132311, 21132313, 21132321, 21132323, 21133112, 21133113, 21133131, 21133211, 21133222, 21133223, 21133232, 21133233, 21133312, 21133313, 21133321, 21133322, 21133331, 21133332, 21211223, 21211321, 21212111, 21212112, 21212122, 21212123, 21212133, 21212212, 21212213, 21212231, 21212233, 21212321, 21212332, 21212333, 21213121, 21213212, 21213223, 21213231, 21213321, 21213332, 21222112, 21231232, 21231233, 21232112, 21232122, 21232132, 21232212, 21232222, 21232231, 21232232, 21232233, 21232321, 21232322, 21232323, 21232332, 21233111, 21233132, 21233212, 21233221, 21233233, 21233312, 21233321, 21311122, 21311223, 21311231, 21311233, 21311311, 21311313, 21311331, 21311333, 21312111, 21312112, 21312122, 21312123, 21312133, 21312211, 21312213, 21312222, 21312223, 21312231, 21312233, 21312311, 21312313, 21312321, 21312322, 21312323, 21312331, 21312332, 21312333, 21313111, 21313112, 21313122, 21313221, 21313231, 21313233, 21313311, 21313312, 21313313, 21313322, 21313331, 21313333, 21331223, 21331332, 21331333, 21332111, 21332112, 21332113, 21332122, 21332131, 21332212, 21332221, 21332223, 21332231, 21332233, 21332312, 21332322, 21332323, 21332331, 21332332, 21332333, 21333111, 21333122, 21333131, 21333132, 21333211, 21333212, 21333221, 21333223, 21333233, 21333312, 21333321, 22313333, 21333333, 22111223, 22111332, 22112111, 22112131, 22112211, 22112223, 22112233, 22112321, 22112323, 22112331, 22112333, 22113111, 22113211, 22113223, 22113232, 22113233, 22113313, 22113323, 22113332, 22131221, 22132112, 22132113, 22132212, 22132231, 22132233, 22132312, 22132323, 22132331, 22133112, 22133211, 22133212, 22133232, 22133312, 22133322, 22133323, 22212111, 22212123, 22212131, 22212212, 22212232, 22212312, 22212321, 22212322, 22212333, 22213111, 22213112, 22213132, 22213212, 22213222, 22213223, 22213312, 22213321, 22222121, 22231221, 22231223, 22231312, 22231322, 22232111, 22232112, 22232121, 22232122, 22232123, 22232212, 22232222, 22232223, 22232232, 22232233, 22232311, 22232312, 22232322, 22232323, 22232331, 22232333, 22233112, 22233211, 22233212, 22233221, 22233222, 22233223, 22233312, 22233323, 22233332, 22311123, 22311212, 22311231, 22311233, 22311331, 22311333, 22312111, 22312123, 22312132, 22312133, 22312211, 22312221, 22312222, 22312223, 22312231, 22312232, 22312233, 22312311, 22312312, 22312322, 22312331, 22312332, 22312333, 22313122, 22313212, 22313221, 22313222, 22313231, 22313232, 22313233, 22313323, 22313331, 22313332, 22323313, 22331123, 22331133, 22331221, 22331223, 22331323, 22331332, 22332112, 22332113, 22332121, 22332123, 22332132, 22332211, 22332221, 22332222, 22332223, 22332232, 22332233, 22332312, 22332321, 22332322, 22332332, 22333112, 22333122, 22333131, 22333132, 22333133, 22333211, 22333212, 22333221, 22333222, 22333223, 22333231, 22333311, 22333313, 22333321, 22333323, 22333332, 23112213, 23112221, 23112223, 23112233, 23112323, 23112333, 23113111, 23113112, 23113121, 23113131, 23113212, 23113311, 23113312, 23113323, 23113332, 23122212, 23131323, 23132111, 23132121, 23132212, 23132221, 23132232, 23132233, 23132311, 23132322, 23132323, 23133112, 23133113, 23133121, 23133233, 23133311, 23133321, 23133331, 23133333, 23211132, 23212112, 23212211, 23212212, 23212221, 23212222, 23212231, 23212332, 23212333, 23213112, 23213121, 23213123, 23213211, 23213212, 23213223, 23213232, 23213311, 23213322, 23213333, 23231233, 23232113, 23232131, 23232211, 23232212, 23232311, 23232323, 23233212, 23233221, 23233231, 23233232, 23233312, 23233333, 23311233, 23311323, 23312112, 23312121, 23312122, 23312123, 23312131, 23312223, 23312311, 23312312, 23312323, 23313111, 23313133, 23313212, 23313222, 23313232, 23313233, 23313323, 23313333, 23331233, 23331323, 23332112, 23332221, 23332222, 23332223, 23332231, 23332311, 23332323, 23332331, 23333111, 23333123, 23333131, 23333211, 23333212, 23333213, 23333222, 23333223, 23333232, 23333233, 23333311, 23333312, 23333323, 31111233, 31112231, 31112333, 31113131, 31113132, 31113222, 31113323, 31113331, 31113332, 31131233, 31132231, 31132232, 31132333, 31133233, 31133331, 31211131, 31211232, 31212112, 31212212, 31212232, 31212321, 31212323, 31212331, 31212332, 31212333, 31213232, 31213233, 31213323, 31213331, 31213332, 31232231, 31232312, 31232333, 31233221, 31233222, 31233233, 31311231, 31311233, 31311332, 31312113, 31312133, 31312212, 31312222, 31312231, 31312233, 31312323, 31312332, 31312333, 31313111, 31313131, 31313132, 31313133, 31313223, 31313232, 31313233, 31313333, 31331331, 31331333, 31332131, 31332133, 31332232, 31332233, 31332312, 31332322, 31332323, 31332333, 31333233, 31333322, 31333332, 31333333, 32111333, 32112212, 32112313, 32112321, 32113131, 32113232, 32113233, 32131133, 32132232, 32132233, 32132331, 32133111, 32133232, 32133233, 32133331, 32211323, 32212133, 32212231, 32212232, 32212233, 32212321, 32212323, 32212332, 32212333, 32213123, 32213132, 32213231, 32213333, 32232131, 32232322, 32232331, 32232333, 32233222, 32233332, 32311131, 32311323, 32312212, 32312231, 32312233, 32312311, 32312322, 32312323, 32312331, 32312332, 32312333, 32313133, 32313231, 32313232, 32313233, 32313313, 32313332, 32313333, 32332133, 32332223, 32332231, 32332232, 32332322, 32332323, 32332331, 32332332, 32332333, 32333223, 32333232, 32333233, 32333312, 32333323, 32333333, 33113111, 33113211, 33113212, 33113233, 33131333, 33133131, 33133333, 33212213, 33212311, 33212333, 33213211, 33213232, 33213333, 33232233, 33232312, 33232333, 33233131, 33233233, 33233333, 33311231, 33312133, 33312322, 33312333, 33313223, 33313233, 33313323, 33313333, 33331232, 33331233, 33331333, 33332131, 33332133, 33332221, 33332232, 33332233, 33332323, 33332333, 33333123, 33333231, 33333232, 33333233, 33333321, and 33333323.

In some embodiments, each segment of the heme domain can have at least 60%, 70%, 80%, 90%, 95%, 96%, 97%, 98%, or 99% or more sequence identity as compared to the reference segment indicated for each of the (segment 1), (segment 2), (segment 3), (segment 4)(segment 5), (segment 6), (segment 7), and (segment 8) of SEQ ID NO:1, SEQ ID NO:2, or SEQ ID NO:3. As discussed herein, the chimeric heme domain is functional when fused to the reductase domain.

In some embodiments, the polypeptide variants can have improved monooxygenase activity compared to the enzyme activity of the wild-type polypeptide of SEQ ID NO:1, 2, or 3.

In some embodiments, the substrate specificity of the polypeptide variants is different as compared to the enzyme activity of the wild-type polypeptide of SEQ ID NO:1, 2, or 3.

In some embodiments, the reference chimeric heme domain can be a chimeric structure selected from:

21112233, 21112331, 21112333, 21113333, 21212233, 21212333, 21311231, 21311233, 21311311, 21311313, 21311331, 21311333, 21312133, 21312211, 21312213, 21312231, 21312311, 21312313, 21312331, 21312332, 21312333, 21313231, 21313233, 21313313, 21313331, 21313333, 22112233, 22112333, 22212333, 22311233, 22311331, 22311333, 22312231, 22312233, 22312331, 22312333, 22313231, 22313233, 22313331, and 22313333.

The cytochrome p450 enzymes described herein may be prepared in various forms, such as lysates, crude extracts, or isolated preparations. The polypeptides can be dissolved in suitable solutions; formulated as powders, such as an acetone powder (with or without stabilizers); or be prepared as lyophilizates. In some embodiments, the cytochrome 0p450 polypeptide can be an isolated polypeptide.

In some embodiments, the isolated cytochrome p450 polypeptide is a substantially pure polypeptide composition. A “substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure polypeptide composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species.

In some embodiments, the fusion polypeptides can be in the form of arrays. The enzymes may be in a soluble form, for example as solutions in the wells of mircotitre plates, or immobilized onto a substrate. The substrate can be a solid substrate or a porous substrate (e.g., membrane), which can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled pore glass (CPG), reverse phase silica or metal, such as gold or platinum. The configuration of a substrate can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location. A plurality of supports can be configured on an array at various locations, addressable for robotic delivery of reagents, or by detection methods and/or instruments.

The present disclosure also provides polynucleotides encoding the engineered cytochrome p450 polypeptides disclosed herein. The polynucleotides may be operatively linked to one or more heterologous regulatory or control sequences that control gene expression to create a recombinant polynucleotide capable of expressing the polypeptide. Expression constructs containing a heterologous polynucleotide encoding the fusion cytochrome p450 enzymes can be introduced into appropriate host cells to express the polypeptide.

Given the knowledge of specific sequences of the cytochrome p450 enzymes, and the specific descriptions of the fusion constructs (e.g., the segment structure of the chimeric heme domains and its fusion to the reductase domains), the amino acid sequence of the engineered cytochrome p450 enzymes will be apparent to the skilled artisan. The knowledge of the codons corresponding to various amino acids coupled with the knowledge of the amino acid sequence of the polypeptides allows those skilled in the art to make different polynucleotides encoding the polypeptides of the disclosure. Thus, the present disclosure contemplates each and every possible variation of the polynucleotides that could be made by selecting combinations based on possible codon choices, and all such variations are to be considered specifically disclosed for any of the polypeptides described herein.

In some embodiments, the polynucleotides comprise polynucleotides that encode the polypeptides described herein but have about 80% or more sequence identity, about 85% or more sequence identity, about 90% or more sequence identity, about 91% or more sequence identity, about 92% or more sequence identity, about 93% or more sequence identity, about 94% or more sequence identity, about 95% or more sequence identity, about 96% or more sequence identity, about 97% or more sequence identity, about 98% or more sequence identity, or about 99% or more sequence identity at the nucleotide level to a reference polynucleotide encoding the cytochrome p450 polypeptides.

In some embodiments, the isolated polynucleotides encoding the polypeptides may be manipulated in a variety of ways to provide for expression of the polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., 2001, Molecular Cloning: A Laboratory Manual, 3rd Ed., Cold Spring Harbor Laboratory Press; and Current Protocols in Molecular Biology, Ausubel. F. ed., Greene Pub. Associates, 1998, updates to 2007.

In some embodiments, the polynucleotides are operatively linked to control sequences for the expression of the polynucleotides and/or polypeptides. In some embodiments, the control sequence may be an appropriate promoter sequence, which can be obtained from genes encoding extracellular or intracellular polypeptides, either homologous or heterologous to the host cell. For bacterial host cells, suitable promoters for directing transcription of the nucleic acid constructs of the present disclosure, include the promoters obtained from the E. coli lac operon, Bacillus subtilis xylA and xylB genes, Bacillus megatarium xylose utilization genes (e.g., Rygus et al., (1991) Appl. Microbiol. Biotechnol. 35:594-599; Meinhardt et al., (1989) Appl. Microbiol. Biotechnol. 30:343-350), prokaryotic beta-lactamase gene (Villa-Kamaroff et al., (1978) Proc. Natl. Acad. Sci. USA 75: 3727-3731), as well as the tac promoter (DeBoer et al., (1983) Proc. Natl. Acad. Sci. USA 80: 21-25). Various suitable promoters are described in “Useful proteins from recombinant bacteria” in Scientific American, 1980, 242:74-94; and in Sambrook et al., supra.

In some embodiments, the control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used.

In some embodiments, the control sequence may also be a suitable leader sequence, a nontranslated region of an mRNA that is important for translation by the host cell. The leader sequence is operably linked to the 5′ terminus of the nucleic acid sequence encoding the polypeptide. Any leader sequence that is functional in the host cell of choice may be used.

In some embodiments, the control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. The foreign signal peptide coding region may be required where the coding sequence does not naturally contain a signal peptide coding region. Effective signal peptide coding regions for bacterial host cells can be the signal peptide coding regions obtained from the genes for Bacillus NClB 11837 maltogenic amylase, Bacillus stearothermophilus alpha-amylase, Bacillus lichenifonnis subtilisin, Bacillus lichenifonnis beta-lactamase, Bacillus stearothermophilus neutral proteases (nprT, nprS, nprM), and Bacillus subtilis prsA. Further signal peptides are described by Simonen and Palva, (1993) Microbiol Rev 57: 109-137.

The present disclosure is further directed to a recombinant expression vector comprising a polynucleotide encoding the engineered cytochrome p450 polypeptides, and one or more expression regulating regions such as a promoter and a terminator, a replication origin, etc., depending on the type of hosts into which they are to be introduced. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon, may be used.

In some embodiments, the expression vector of the present disclosure preferably contains one or more selectable markers, which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus lichenifonnis, or markers, which confer antibiotic resistance such as ampicillin, kanamycin, chloramphenicol (Example 1) or tetracycline resistance. Other useful markers will be apparent to the skilled artisan.

In another aspect, the present disclosure provides a host cell comprising a polynucleotide encoding the fusion cytochrome p450 polypeptides, the polynucleotide being operatively linked to one or more control sequences for expression of the fusion polypeptide in the host cell. Host cells for use in expressing the fusion polypeptides encoded by the expression vectors of the present disclosure are well known in the art and include but are not limited to, bacterial cells, such as E. coli and Bacillus megaterium; insect cells such as Drosophila S2 and Spodoptera Sf9 cells; animal cells such as CHO, COS, BHK, 293, and Bowes melanoma cells; and plant cells. Other suitable host cells will be apparent to the skilled artisan. Appropriate culture mediums and growth conditions for the above-described host cells are well known in the art.

The cytochrome p450 polypeptides of the present disclosure can be made by using methods well known in the art. Polynucleotides can be synthesized by recombinant techniques, such as that provided in Sambrook et al., 2001, Molecular Cloning: A Laboratory Manual, 3rd Ed., Cold Spring Harbor Laboratory Press; and Current Protocols in Molecular Biology, Ausubel. F. ed., Greene Pub. Associates, 1998, updates to 2007. Polynucleotides encoding the enzymes, or the primers for amplification can also be prepared by standard solid-phase methods, according to known synthetic methods, for example using phosphoramidite method described by Beaucage et al., (1981) Tet Lett 22:1859-69, or the method described by Matthes et al., (1984) EMBO J. 3:801-05, e.g., as it is typically practiced in automated synthetic methods. In addition, essentially any nucleic acid can be obtained from any of a variety of commercial sources, such as The Midland Certified Reagent Company, Midland, Tex., The Great American Gene Company, Ramona, Calif., ExpressGen Inc. Chicago, Ill., Operon Technologies Inc., Alameda, Calif., and many others.

Engineered enzymes expressed in a host cell can be recovered from the cells and or the culture medium using any one or more of the well known techniques for protein purification, including, among others, lysozyme treatment, sonication, filtration, salting-out, ultra-centrifugation, chromatography, and affinity separation (e.g., substrate bound antibodies). Suitable solutions for lysing and the high efficiency extraction of proteins from bacteria, such as E. coli, are commercially available under the trade name CelLytic BTM from Sigma-Aldrich of St. Louis Mo.

Chromatographic techniques for isolation of the polypeptides include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

Descriptions of SCHEMA directed recombination and synthesis of chimeric heme domains and reductase domains are described in the examples herein, as well as in Otey et al., (2006), PLoS Biol. 4(5):e112; Meyer et al., (2003) Protein Sci., 12:1686-1693; U.S. patent application Ser. No. 12/024,515, filed Feb. 1, 2008; and U.S. patent application Ser. No. 12/027,885, filed Feb. 7, 2008; all publications incorporated herein by reference in their entirety.

As discussed above, the fusion polypeptide can be used in a variety of applications, such as, among others, transformation of pharmaceutical compounds to generate active metabolites, conversion of alkyl substrates to their corresponding alcohols, and conversion of compounds to generate intermediates for the synthesis of pharmaceutical compounds. In these methods, the fusion polypeptide is contacted with the substrate compound, or candidate substrate, under suitable conditions, such as in the presence of a cofactor (e.g., NADH or NADPH, as provided in the examples) to cause insertion of one atom of oxygen into an organic substrate.

The following examples are meant to further explain, but not limited the foregoing disclosure or the appended claims.

### EXAMPLES

Thermostability measurements. Cell extracts were prepared and P450 concentrations were determined as reported previously. Cell extract samples containing 4 μM of P450 were heated in a thermocycler over a range of temperatures for 10 minutes followed by rapid cooling to 4° C. for 1 minute. The precipitate was removed by centrifugation. The P450 remaining in the supernatant was measured by CO-difference spectroscopy. T₅₀, the temperature at which 50 percent of protein irreversibly denatured after a 10-min incubation, was determined by fitting the data to a two-state denaturation model. To check the variability and reproducibility of the measurement, four parallel independent experiments (from cell culture to T₅₀ measurement) were conducted on A2, which yielded an average T₅₀ of 43.6° C. and a standard deviation (σ_{M}) of 1.0° C. For some sequences, T₅₀ s were measured twice, and the average of all the measurements was used in the analysis.

Properly folded heme domains were identified based upon CO-binding. Polypeptides were incubated in a CO tank for 10 minutes and the light absorbance between 400 and 500 nm was measured. The presence of a feature peak at 450 nm indicates correct heme binding and thus a properly folded P450 heme protein.

Linear regression. The linear model

$${T}_{50} = {a}_{0} + {\sum }_{i} {\sum }_{j} {a}_{ij} {x}_{ij}$$

was used for regression, where T₅₀ is the dependent variable and fragments xᵢⱼ (from the i^{th} position and j^{th} parent, where i=1, 2, . . . 8 and j=1 or 3) are the independent variables. The were dummy-coded, such that if a chimera took fragment 1 from parent 1, x₁₁=1 and x₁₃=0. Parent A2 was used as the reference for all eight fragments, so the constant term (a₀) is the predicted T₅₀ of A2. The thermostability contribution of each fragment relative to the corresponding A2 fragment is given by the regression coefficient aᵢⱼ. Regression was performed using SPSS (SPSS for Windows, Rel. 11.0.1. 2001. Chicago: SPSS Inc.).

Construction of chimeric cytochrome P450s. To generate a library of CYP102A sequences for these applications, a structure-guided SCHEMA recombination of the heme domains of CYP102A1 and its homologs CYP102A2 (A2) and CYP102A3 (A3) was used to create an extensive library of properly folded and catalytically active enzymes. The folded chimeras exhibit a great deal of sequence diversity, differing from the closest parent sequence by an average of 72 amino acid substitutions. Some of these chimeric P450s were shown to be more stable than any of the parents.

The SCHEMA library was constructed by site-directed recombination at seven crossover sites, so that a chimeric P450 sequence is made up of eight fragments, each chosen from one of the three parents. As such, the chimeria are presented herein as an 8-digit number, where each digit indicates the parent from which each of the eight blocks was inherited. The thermostabilities of a subset of the folded chimeras were measured and analyzed the relationship between sequence and stability. Based on these analyses, chimeras were predicted, constructed and characterized.

To construct a given stable chimera, two chimeras having parts of the targeted gene (e.g. 21311212 and 11312333 for the target chimera 21312333) were selected as templates. The target gene was constructed by overlap extension PCR, cloned into the pCWori expression vector, and transformed into the catalase-free E. coli strain SN0037. All constructs were confirmed by sequencing.

Enzyme activity assay. Activity on 2-phenoxyethanol was analyzed in 96-well plates using the 4-aminoantipyrine (4-AAP) assay. 80 μl of P450 chimera (4 μM) was mixed 20 μl of 2-phenoxyethanol (3 M) in each well. The reaction was initiated by adding 20 μl of 120 mM hydrogen peroxide. The reaction mixture was incubated at room temperature for two hours. Then 50 μl of basic buffer (0.2 M NaOH and 4 M Urea) was added into the reaction mixture to raise the pH for the 4-AAP assay. 25 μl of 0.6% 4-AAP was added, the reading at 500 nm was taken for zeroing, and then 25 μl of 0.6% potassium persulfate was added. After incubation of 10 minutes at room temperature, the absorbance at 500 nm was recorded. The total turnover number (TTN) was calculated and then normalized to the most active parent, A1.

Protein stabilization by additivity of fragment contributions. Linear regression model parameters obtained from 205 T₅₀ measurements were used to predict T₅₀ values for 6,561 chimeras in the SCHEMA P450 library. A significant number (˜300) of chimeras are predicted to be more stable than the most stable parent. Those with predicted T₅₀ values greater or equal to 60° C. (total of 31) were stable, with a T₅₀ between 58.5° C. and 64.4° C. (Table 1).

TABLE 1
A stabilized cytochrome P450 heme domain family.

| | Predicted | Measured | | | Predicted | Measured | |
|---|---|---|---|---|---|---|---|
| Sequence | T₅₀(C.) | T₅₀(C.) | Activity³ | Sequence | T₅₀(C.) | T₅₀(C.) | Activity |
| 21312333^{1,2} | 63.8 | 64.4 | 1.0 | 21311231¹ | 60.7 | 63.2 | 0.8 |
| 21312331^{1,2} | 62.8 | 60.6 | 3.1 | 22312313¹ | 60.6 | 61.0 | 2.5 |
| 21311333¹ | 62.8 | 59.2 | 2.5 | 21313313¹ | 60.6 | 61.9 | 4.7 |
| 21312233^{1,2} | 62.7 | 63.1 | 0.6 | 22311331¹ | 60.4 | 58.9 | 5.1 |
| 22312333^{1,2} | 62.4 | 63.5 | 1.9 | 21312133¹ | 60.4 | 60.1 | 2.8 |
| 21313333^{1,2} | 62.4 | 62.9 | 3.8 | 22312231¹ | 60.3 | 61.4 | 2.3 |
| 21312313¹ | 62.0 | 62.2 | 2.8 | 21313231¹ | 60.3 | 61.0 | 1.8 |
| 21311331¹ | 61.8 | 62.9 | 1.0 | 22311233¹ | 60.3 | 60.9 | 3.1 |
| 21312231^{1,2} | 61.7 | 62.8 | 1.0 | 21311311¹ | 60.1 | 61.0 | 3.2 |
| 21311233¹ | 61.7 | 62.7 | 0.7 | 22313331¹ | 60.0 | 58.5 | 7.2 |
| 21313331¹ | 61.4 | 62.2 | 5.5 | 21312211¹ | 60.0 | 59.3 | 2.8 |
| 22312331¹ | 61.4 | 59.3 | 5.1 | 21212333² | 59.6 | 63.2 | 0.4 |
| 22311333¹ | 61.4 | 60.1 | 4.7 | 21112333² | 59.5 | 61.6 | 1.1 |
| 22312233^{1,2} | 61.3 | 61.0 | 2.7 | 21212233² | 58.5 | 60.0 | 1.3 |
| 21313233^{1,2} | 61.2 | 60.0 | 3.3 | 21112331² | 58.5 | 61.6 | 0.6 |
| 21312311¹ | 61.1 | 59.1 | 3.0 | 21112233² | 58.4 | 58.7 | 0.7 |
| 22313333¹ | 61.0 | 64.3 | 9.0 | 22212333² | 58.2 | 58.2 | 3.2 |
| 21311313¹ | 61.0 | 61.2 | 2.7 | 22112333² | 58.1 | 58.0 | 4.2 |
| 21312213¹ | 60.9 | 60.6 | 1.1 | 21113333² | 58.1 | 61.0 | 4.1 |
| 21312332¹ | 60.8 | 59.9 | 1.3 | 22112233² | 57.0 | 58.7 | 5.2 |

¹predicted to be highly stable by linear regression;
²predicted to be stable by consensus analysis;
³activity on 2-phenoxyethanol is reported as total turnover number normalized to the most active parent protein, A1.

Protein stabilization by consensus. Most stable chimeras were predicted based on consensus energies for 6,561 chimeras in the library; the 20 with the lowest consensus energies are listed in Table 2. Due to bias in the library construction, the data set of 955 chimeras has very few representatives of A2 at position 4, preventing accurate assessment of this fragment's thermostability contribution. Three sequences with this fragment were not constructed; the remaining seventeen were constructed. The sequence with consensus fragments at all eight positions (21312333) and therefore the lowest consensus energy is the “consensus sequence”, and should be the most stable chimera. Indeed, the consensus sequence has the highest measured stability among all 239 chimeras with known T₅₀ and is also the MTP predicted by the linear regression model.

TABLE 2
The 20 chimeras with lowest total consensus energies.

| | | | Consensus |
|---|---|---|---|
| Sequence | Consensus energy | Sequence | energy |
| 21312333 | −3.40 | 22312233 | −3.10 |
| 21312233 | −3.35 | 21322233 | −3.07 |
| 21112333 | −3.29 | 21313233 | −3.06 |
| 21212333 | −3.24 | 21312231 | −3.04 |
| 21112233 | −3.24 | 22112333 | −3.04 |
| 21212233 | −3.18 | 21122333 | −3.01 |
| 22312333 | −3.15 | 21113333 | −3.00 |
| 21322333 | −3.13 | 21112331 | −2.99 |
| 21313333 | −3.12 | 22212333 | −2.98 |
| 21312331 | −3.10 | 22112233 | −2.98 |

The protein expression levels of most of the thermostable chimeras were higher than those of the parent proteins. Most thermostable chimeras expressed well even without the inducing agent isopropyl-beta-D-thiogalactopyranoside (IPTG).

Substrate specificity of heme-reductase fusion polypeptides: To explore further the activity of chimeric heme domains, seventeen proteins, including the three parent heme domains, were chosen for holoenzyme construction by fusion to a wildtype CYP102A reductase domain. For each sequence, four proteins were examined—the heme domain and its fusion to each of the three reductase domains—for a total of 68 constructs. Heme domains contain the first 463 amino acids for A1 and the first 466 amino acids for A2 and A3. The reductase domains start at amino acid E464 for R1, K467 for R2 and D467 for R3 and encode the linker region of the corresponding reductase.

The chimeric sequences are reported in terms of the parent from which each of the eight sequence blocks is inherited (Table 3). Twelve of the fourteen chimeras were selected because they displayed relatively high activities on substrates in preliminary studies. Chimera 23132233 was chosen because it displayed low peroxygenase activity, while 22312333 was selected because it is more thermostable than any of the parents (T₅₀=62° C.). For the constructs studied here, the reductase identity is indicated as the ninth sequence element, with R0 referring to no reductase (i.e., heme domain peroxygenase).

TABLE 3
Pairwise correlations of normalized activities for
monooxygenases (R1, R2, R3) and peroxygenases (R0) of fourteen
chimeras and the A1 and A2 parents. R₂ values are reported. Bold
and underlined = 0.7-1.0; Underlined = 0.4-0.7; Regular = 0.0-0.4.

| Heme sequence | R0/R1 | R0/R2 | R0/R3 | R1/R2 | R1/R3 | R2/R3 |
|---|---|---|---|---|---|---|
| 11111111 | 0.49 | 0.00 | 0.53 | 0.21 | 0.66 | 0.11 |
| 22222222 | 0.70 | 0.53 | 0.49 | 0.75 | 0.83 | 0.66 |
| 11113311 | 0.61 | 0.65 | 0.49 | 0.90 | 0.59 | 0.78 |
| 12112333 | 0.11 | 0.04 | 0.00 | 0.91 | 0.11 | 0.10 |
| 21113312 | 0.14 | 0.01 | 0.00 | 0.73 | 0.76 | 0.77 |
| 21313111 | 0.24 | 0.19 | 0.05 | 0.84 | 0.15 | 0.39 |
| 21313311 | 0.25 | 0.28 | 0.00 | 0.41 | 0.01 | 0.34 |
| 21333233 | 0.90 | 0.64 | 0.87 | 0.72 | 0.95 | 0.66 |
| 22132231 | 0.80 | 0.85 | 0.56 | 0.98 | 0.64 | 0.60 |
| 22213132 | 0.46 | 0.08 | 0.37 | 0.11 | 0.01 | 0.54 |
| 22312333 | 0.01 | 0.02 | 0.00 | 0.69 | 0.69 | 0.25 |
| 22313233 | 0.17 | 0.01 | 0.08 | 0.02 | 0.85 | 0.07 |
| 23132233 | 0.96 | 0.89 | 0.97 | 0.90 | 0.99 | 0.90 |
| 32312231 | 0.14 | 0.06 | 0.02 | 0.07 | 0.04 | 0.21 |
| 32312333 | 0.33 | 0.41 | 0.02 | 0.97 | 0.40 | 0.33 |
| 32313233 | 0.15 | 0.44 | 0.09 | 0.74 | 0.60 | 0.38 |

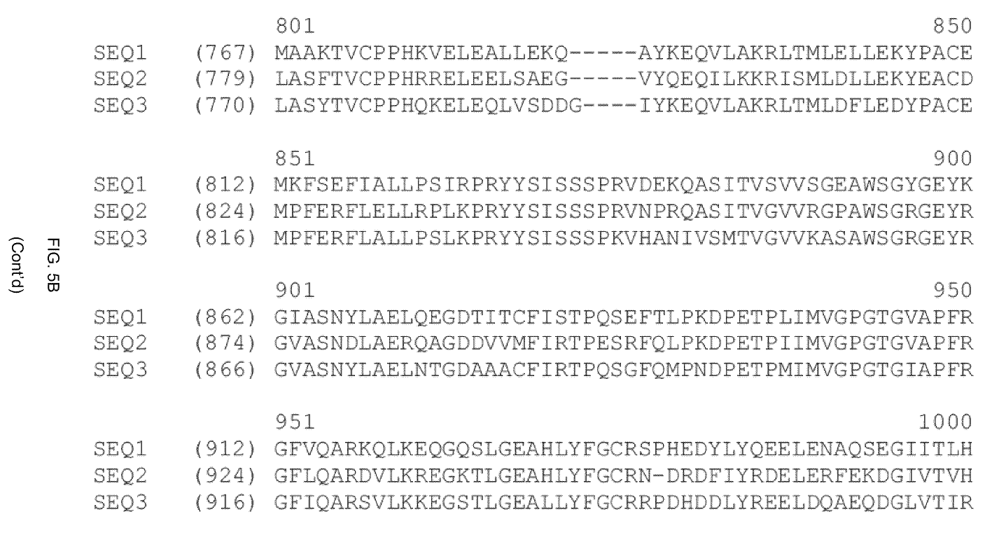

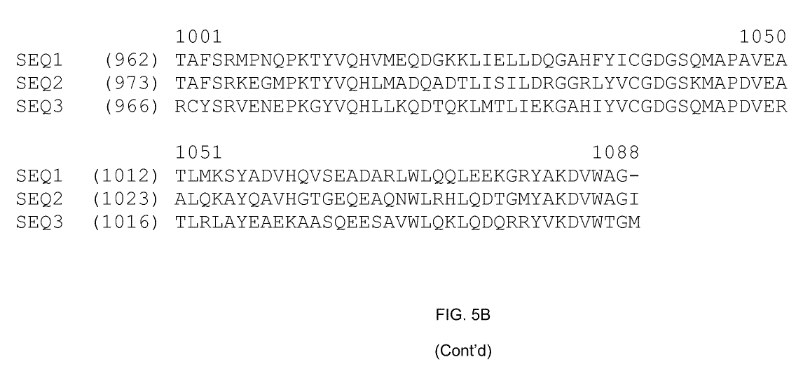

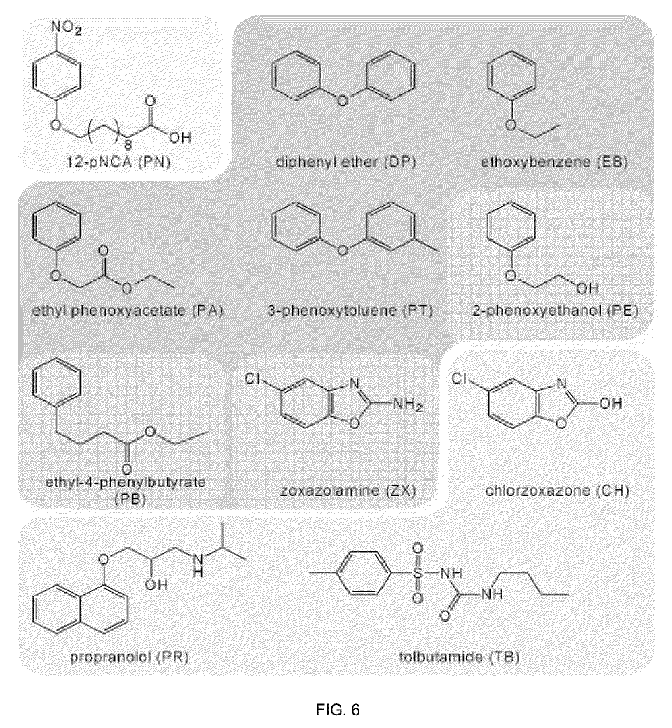

To assess the functional diversity of the chimeric P450s, their activities were measured on the eleven substrates shown in FIG. 6. Propranolol (PR), tolbutamide (TB) and chlorzoxazone (CH) are drugs that are metabolized by human P450s. 12-p-nitrophenoxycarboxylic acid (PN) is a long-chain fatty acid surrogate; parent A1-R1 holoenzyme and the A1 heme domain (with the F87A mutation) both show high activity on this substrate. Previous work showed that A1 has weak peroxygenase activity on some of the aromatic substrates. Aromatic hydroxylation products of all substrates can be detected quantitatively using the 4-amino antipyrine assay. PN hydroxylation can be monitored spectrophometrically.

Peroxygenase activities of the 16 heme domains (all except A3) were determined by assaying for product formation after a fixed reaction time in 96-well plates. Similar assays were used to determine monooxygenase activities for each of the fusion proteins. Final enzyme concentrations were fixed to 1 μM in order to reduce large errors associated with low expression and to allow us to compare chimera activities using absorbance values directly. Protein concentrations were re-assayed in 96-well format and determined to be 0.88 μM+/−13% (SD/average). All samples were prepared and analyzed in triplicate, and outlier data points were eliminated. Tables 4 and Table 5 report the averages and standard deviations for each of the assays. More than 85% of the data for each substrate was retained, and more than 95% was retained for 6 of the 11 substrates (Table 10).

TABLE 4
Average activity in absorbance units for each substrate-construct
pair (maximal value for each substrate in bold/italic).

| | 2-phenoxyethanol | ethoxybenzene | ethyl phenoxyacetate | 3-phenoxytoluene | ethyl 4-phenylbutyrate |
|---|---|---|---|---|---|
| 11111111-R0 | 0.105 | 0.000 | 0.000 | 0.000 | 0.013 |
| 11111111-R1 | 0.152 | 0.115 | 0.136 | 0.053 | 0.202 |
| 11111111-R2 | 0.434 | 0.179 | 0.157 | 0.113 | 0.200 |
| 11111111-R3 | 0.048 | 0.000 | 0.038 | 0.000 | 0.059 |
| 22222222-R0 | 0.054 | 0.000 | 0.000 | 0.000 | 0.013 |
| 22222222-R1 | 0.042 | 0.000 | 0.038 | 0.000 | 0.027 |
| 22222222-R2 | 0.039 | 0.000 | 0.045 | 0.000 | 0.027 |
| 22222222-R3 | 0.065 | 0.000 | 0.040 | 0.000 | 0.048 |
| 33333333-R3 | 0.049 | 0.000 | 0.033 | 0.000 | 0.046 |
| 11113311-R0 | 0.463 | 0.000 | 0.046 | 0.000 | 0.011 |
| 11113311-R1 | 0.448 | 0.236 | 0.160 | 0.072 | 0.135 |
| 11113311-R2 | 0.329 | 0.145 | 0.087 | 0.000 | 0.091 |
| 11113311-R3 | 0.118 | 0.000 | 0.033 | 0.000 | 0.032 |
| 12112333-R0 | 0.544 | 0.053 | 0.048 | 0.000 | 0.013 |
| 12112333-R1 | 0.513 | 0.262 | 0.163 | 0.091 | 0.124 |
| 12112333-R2 | 0.511 | 0.334 | 0.163 | 0.116 | 0.135 |
| 12112333-R3 | 0.129 | 0.044 | 0.039 | 0.000 | 0.043 |
| 21113312-R0 | 0.522 | 0.135 | 0.078 | 0.000 | 0.017 |
| 21113312-R1 | 0.269 | 0.107 | 0.084 | 0.000 | 0.063 |
| 21113312-R2 | 0.213 | 0.085 | 0.073 | 0.046 | 0.066 |
| 21113312-R3 | 0.179 | 0.063 | 0.058 | 0.000 | 0.049 |
| 21313111-R0 | 0.731 | 0.105 | 0.073 | 0.000 | 0.016 |
| 21313111-R1 | 0.617 | 0.313 | 0.173 | 0.167 | 0.059 |
| 21313111-R2 | 0.660 | 0.282 | 0.139 | 0.162 | 0.102 |
| 21313111-R3 | 0.767 | 0.256 | 0.258 | 0.207 | [symbol] |
| 21313311-R0 | 0.365 | 0.000 | 0.046 | 0.000 | 0.009 |
| 21313311-R1 | 0.343 | 0.002 | 0.109 | 0.061 | 0.089 |
| 21313311-R2 | 0.305 | 0.074 | 0.092 | 0.000 | 0.086 |
| 21313311-R3 | 0.190 | 0.109 | 0.096 | 0.097 | 0.115 |
| 21333233-R0 | 0.113 | 0.000 | 0.036 | 0.000 | 0.020 |
| 21333233-R1 | 0.046 | 0.000 | 0.035 | 0.000 | 0.029 |
| 21333233-R2 | 0.180 | 0.104 | 0.119 | 0.000 | 0.070 |
| 21333233-R3 | 0.057 | 0.000 | 0.035 | 0.000 | 0.036 |
| 22132231-R0 | 0.034 | 0.000 | 0.000 | 0.000 | 0.009 |
| 22132231-R1 | 0.025 | 0.000 | 0.024 | 0.000 | 0.023 |
| 22132231-R2 | 0.045 | 0.000 | 0.035 | 0.000 | 0.026 |
| 22132231-R3 | 0.022 | 0.000 | 0.000 | 0.000 | 0.016 |
| 22213132-R0 | 0.259 | 0.051 | 0.061 | 0.000 | 0.010 |
| 22213132-R1 | 0.584 | 0.217 | 0.236 | 0.076 | 0.061 |
| 22213132-R2 | 0.277 | 0.289 | 0.253 | 0.169 | 0.153 |
| 22213132-R3 | 0.172 | 0.070 | 0.077 | 0.000 | 0.038 |
| 22312333-R0 | 0.103 | 0.000 | 0.024 | 0.000 | 0.008 |
| 22312333-R1 | 0.080 | 0.000 | 0.044 | 0.000 | 0.056 |
| 22312333-R2 | 0.172 | 0.067 | 0.064 | 0.049 | 0.121 |
| 22312333-R3 | 0.034 | 0.000 | 0.000 | 0.000 | 0.022 |
| 22313233-R0 | 0.185 | 0.000 | 0.050 | 0.000 | 0.011 |
| 22313233-R1 | 0.064 | 0.000 | 0.036 | 0.000 | 0.033 |
| 22313233-R2 | 0.260 | 0.204 | 0.150 | 0.187 | 0.089 |
| 22313233-R3 | 0.077 | 0.000 | 0.041 | 0.000 | 0.034 |
| 23132233-R0 | 0.024 | 0.000 | 0.000 | 0.000 | 0.019 |
| 23132233-R1 | 0.044 | 0.000 | 0.049 | 0.000 | 0.051 |
| 23132233-R2 | 0.049 | 0.000 | 0.055 | 0.046 | 0.054 |
| 23132233-R3 | 0.030 | 0.000 | 0.031 | 0.000 | 0.034 |
| 32312231-R0 | 0.354 | 0.065 | 0.065 | 0.000 | 0.016 |
| 32312231-R1 | 0.067 | 0.053 | 0.055 | 0.000 | 0.051 |
| 32312231-R2 | 0.204 | 0.245 | 0.277 | 0.154 | 0.090 |
| 32312231-R3 | 0.064 | 0.000 | 0.035 | 0.000 | 0.025 |
| 32312333-R0 | [symbol] | 0.338 | 0.236 | 0.076 | 0.025 |
| 32312333-R1 | 1.000 | [symbol] | [symbol] | [symbol] | 0.167 |
| 32312333-R2 | 0.907 | 0.712 | 0.553 | 0.245 | 0.133 |
| 32312333-R3 | 0.212 | 0.189 | 0.264 | 0.178 | 0.066 |
| 32313233-R0 | 0.796 | 0.363 | 0.276 | 0.095 | 0.036 |
| 32313233-R1 | 0.249 | 0.471 | 0.476 | 0.280 | 0.163 |
| 32313233-R2 | 0.585 | 0.566 | 0.454 | 0.197 | 0.153 |
| 32313233-R3 | 0.147 | 0.123 | 0.125 | 0.081 | 0.056 |

| | diphenyl ether | 2-amino-5-chloro-benzoxazole | propranolol | chloroxazone | tolbutamide | 12-pNCA |
|---|---|---|---|---|---|---|
| 11111111-R0 | 0.027 | 0.000 | 0.011 | 0.013 | 0.011 | 0.170 |
| 11111111-R1 | 0.177 | 0.055 | 0.037 | 0.032 | 0.033 | 0.302 |
| 11111111-R2 | 0.114 | 0.146 | 0.029 | 0.025 | 0.029 | 0.114 |
| 11111111-R3 | 0.030 | 0.054 | 0.023 | 0.019 | 0.022 | 0.132 |
| 22222222-R0 | 0.009 | 0.000 | 0.010 | 0.014 | 0.011 | 0.026 |
| 22222222-R1 | 0.031 | 0.020 | 0.021 | 0.015 | 0.028 | 0.064 |
| 22222222-R2 | 0.083 | 0.022 | 0.020 | 0.016 | 0.018 | 0.037 |
| 22222222-R3 | 0.031 | 0.055 | 0.028 | 0.024 | 0.024 | 0.079 |
| 33333333-R3 | 0.026 | 0.066 | 0.030 | 0.022 | 0.024 | 0.069 |
| 11113311-R0 | 0.031 | 0.000 | 0.013 | 0.012 | 0.009 | 0.190 |
| 11113311-R1 | 0.225 | 0.061 | 0.029 | 0.028 | 0.027 | [symbol] |
| 11113311-R2 | 0.159 | 0.051 | 0.030 | 0.024 | 0.024 | 0.277 |
| 11113311-R3 | 0.028 | 0.047 | 0.022 | 0.017 | 0.019 | 0.155 |
| 12112333-R0 | 0.036 | 0.000 | 0.012 | 0.014 | 0.013 | 0.056 |
| 12112333-R1 | 0.414 | 0.038 | 0.020 | 0.017 | 0.019 | 0.170 |
| 12112333-R2 | 0.462 | 0.063 | 0.025 | 0.024 | 0.025 | 0.143 |
| 12112333-R3 | 0.058 | 0.080 | 0.025 | 0.019 | 0.022 | 0.053 |
| 21113312-R0 | 0.034 | 0.000 | 0.017 | 0.017 | 0.013 | 0.069 |
| 21113312-R1 | 0.056 | 0.045 | 0.038 | 0.045 | 0.034 | 0.065 |
| 21113312-R2 | 0.047 | 0.055 | 0.033 | 0.038 | 0.031 | 0.050 |
| 21113312-R3 | 0.034 | 0.075 | 0.034 | 0.037 | 0.033 | 0.031 |
| 21313111-R0 | 0.056 | 0.000 | 0.018 | 0.012 | 0.013 | 0.000 |
| 21313111-R1 | 0.370 | 0.044 | 0.024 | 0.024 | 0.024 | 0.033 |
| 21313111-R2 | 0.332 | 0.079 | 0.029 | 0.027 | 0.028 | 0.000 |
| 21313111-R3 | 0.516 | 0.137 | 0.102 | 0.039 | 0.076 | 0.000 |
| 21313311-R0 | 0.036 | 0.000 | 0.012 | 0.011 | 0.012 | 0.000 |
| 21313311-R1 | 0.202 | 0.017 | 0.019 | 0.015 | 0.019 | 0.000 |
| 21313311-R2 | 0.149 | 0.050 | 0.030 | 0.029 | 0.029 | 0.000 |
| 21313311-R3 | 0.150 | 0.135 | 0.072 | 0.071 | 0.060 | 0.000 |
| 21333233-R0 | 0.016 | 0.023 | 0.025 | 0.020 | 0.020 | 0.000 |
| 21333233-R1 | 0.026 | 0.022 | 0.024 | 0.019 | 0.022 | 0.000 |
| 21333233-R2 | 0.090 | 0.039 | 0.035 | 0.034 | 0.031 | 0.062 |
| 21333233-R3 | 0.025 | 0.040 | 0.026 | 0.025 | 0.024 | 0.000 |
| 22132231-R0 | 0.006 | 0.000 | 0.005 | 0.006 | 0.007 | 0.000 |
| 22132231-R1 | 0.016 | 0.000 | 0.018 | 0.014 | 0.018 | 0.000 |
| 22132231-R2 | 0.033 | 0.000 | 0.018 | 0.015 | 0.020 | 0.000 |
| 22132231-R3 | 0.015 | 0.025 | 0.014 | 0.012 | 0.015 | 0.000 |
| 22213132-R0 | 0.017 | 0.020 | 0.010 | 0.019 | 0.013 | 0.000 |
| 22213132-R1 | 0.172 | 0.068 | 0.031 | 0.040 | 0.030 | 0.133 |
| 22213132-R2 | 0.206 | 0.152 | [symbol] | [symbol] | [symbol] | 0.000 |
| 22213132-R3 | 0.043 | 0.051 | 0.026 | 0.025 | 0.024 | 0.015 |
| 22312333-R0 | 0.017 | 0.000 | 0.009 | 0.006 | 0.009 | 0.000 |
| 22312333-R1 | 0.132 | 0.002 | 0.015 | 0.015 | 0.018 | 0.000 |
| 22312333-R2 | 0.356 | 0.117 | 0.019 | 0.012 | 0.017 | 0.000 |
| 22312333-R3 | 0.019 | 0.000 | 0.012 | 0.011 | 0.015 | 0.000 |
| 22313233-R0 | 0.029 | 0.000 | 0.000 | 0.009 | 0.010 | 0.000 |
| 22313233-R1 | 0.044 | 0.023 | 0.021 | 0.016 | 0.021 | 0.000 |
| 22313233-R2 | 0.415 | 0.049 | 0.022 | 0.016 | 0.019 | 0.000 |
| 22313233-R3 | 0.031 | 0.053 | 0.026 | 0.020 | 0.023 | 0.000 |
| 23132233-R0 | 0.019 | 0.022 | 0.025 | 0.021 | 0.021 | 0.000 |
| 23132233-R1 | 0.037 | 0.035 | 0.042 | 0.039 | 0.036 | 0.000 |
| 23132233-R2 | 0.044 | 0.043 | 0.043 | 0.041 | 0.030 | 0.000 |
| 23132233-R3 | 0.024 | 0.025 | 0.031 | 0.026 | 0.020 | 0.000 |
| 32312231-R0 | 0.057 | 0.000 | 0.015 | 0.013 | 0.010 | 0.000 |
| 32312231-R1 | 0.156 | 0.063 | 0.021 | 0.016 | 0.021 | 0.000 |
| 32312231-R2 | 0.448 | 0.063 | 0.019 | 0.016 | 0.020 | 0.139 |
| 32312231-R3 | 0.024 | 0.044 | 0.018 | 0.015 | 0.016 | 0.048 |
| 32312333-R0 | 0.297 | 0.067 | 0.018 | 0.019 | 0.019 | 0.000 |
| 32312333-R1 | 0.664 | 0.233 | 0.022 | 0.046 | 0.023 | 0.034 |
| 32312333-R2 | 0.538 | 0.174 | 0.018 | 0.023 | 0.022 | 0.044 |
| 32312333-R3 | 0.561 | 0.145 | 0.023 | 0.023 | 0.023 | 0.000 |
| 32313233-R0 | 0.389 | 0.121 | 0.009 | 0.023 | 0.023 | 0.000 |
| 32313233-R1 | [symbol] | [symbol] | 0.044 | 0.048 | 0.039 | 0.018 |
| 32313233-R2 | 0.465 | 0.229 | 0.029 | 0.037 | 0.029 | 0.017 |
| 32313233-R3 | 0.304 | 0.153 | 0.034 | 0.032 | 0.031 | 0.000 |

TABLE 5
Standard deviations/average of absorbance for each substrate construct
pair. Blanks indicate where the average absorbance equals zero.

| | 2-phenoxyethanol | ethoxybenzene | ethyl phenoxyacetate | 3-phenoxytoluene | ethyl 4-phenylbutyrate |
|---|---|---|---|---|---|
| 11111111-R0 | 0.091 | | | | 0.233 |
| 11111111-R1 | 0.093 | 0.163 | 0.058 | 0.128 | 0.033 |
| 11111111-R2 | 0.039 | 0.020 | 0.118 | 0.135 | 0.041 |
| 11111111-R3 | 0.054 | | 0.031 | | 0.029 |
| 22222222-R0 | 0.089 | | | | 0.156 |
| 22222222-R1 | 0.128 | | 0.074 | | 0.077 |
| 22222222-R2 | 0.071 | | 0.054 | | 0.113 |
| 22222222-R3 | 0.053 | | 0.111 | | 0.084 |
| 33333333-R3 | 0.134 | | 0.126 | | 0.017 |
| 11113311-R0 | 0.092 | | 0.097 | | 0.086 |
| 11113311-R1 | 0.045 | 0.158 | 0.124 | 0.092 | 0.159 |
| 11113311-R2 | 0.045 | 0.018 | 0.113 | | 0.035 |
| 11113311-R3 | 0.105 | | 0.093 | | 0.033 |
| 12112333-R0 | 0.012 | 0.046 | 0.045 | | 0.159 |
| 12112333-R1 | 0.092 | 0.014 | 0.114 | 0.107 | 0.029 |
| 12112333-R2 | 0.054 | 0.118 | 0.094 | 0.021 | 0.024 |
| 12112333-R3 | 0.039 | 0.016 | 0.057 | | 0.020 |
| 21113312-R0 | 0.129 | 0.076 | 0.126 | | 0.074 |
| 21113312-R1 | 0.065 | 0.049 | 0.060 | | 0.045 |
| 21113312-R2 | 0.024 | 0.190 | 0.114 | 0.150 | 0.064 |
| 21113312-R3 | 0.094 | 0.147 | 0.067 | | 0.051 |
| 21313111-R0 | 0.078 | 0.177 | 0.142 | | 0.038 |
| 21313111-R1 | 0.116 | 0.046 | 0.019 | 0.088 | 0.055 |
| 21313111-R2 | 0.012 | 0.084 | 0.076 | 0.039 | 0.037 |
| 21313111-R3 | 0.038 | 0.200 | 0.092 | 0.034 | 0.034 |
| 21313311-R0 | 0.065 | | 0.143 | | 0.162 |
| 21313311-R1 | 0.026 | 0.051 | 0.166 | 0.178 | 0.086 |
| 21313311-R2 | 0.137 | 0.141 | 0.169 | | 0.018 |
| 21313311-R3 | 0.012 | 0.053 | 0.038 | 0.075 | 0.010 |
| 21333233-R0 | 0.062 | | 0.242 | | 0.110 |
| 21333233-R1 | 0.095 | | 0.049 | | 0.038 |
| 21333233-R2 | 0.036 | 0.183 | 0.135 | | 0.016 |
| 21333233-R3 | 0.043 | | 0.044 | | 0.044 |
| 22132231-R0 | 0.002 | | | | 0.180 |
| 22132231-R1 | 0.052 | | 0.041 | | 0.051 |
| 22132231-R2 | 0.063 | | 0.067 | | 0.019 |
| 22132231-R3 | 0.080 | | | | 0.061 |
| 22213132-R0 | 0.153 | 0.128 | 0.058 | | 0.081 |
| 22213132-R1 | 0.077 | 0.118 | 0.104 | 0.053 | 0.066 |
| 22213132-R2 | 0.065 | 0.091 | 0.059 | 0.075 | 0.050 |
| 22213132-R3 | 0.097 | 0.061 | 0.116 | | 0.061 |
| 22312333-R0 | 0.023 | | 0.173 | | 0.181 |
| 22312333-R1 | 0.103 | | 0.110 | | 0.046 |
| 22312333-R2 | 0.060 | 0.191 | 0.108 | 0.050 | 0.047 |
| 22312333-R3 | 0.101 | | | | 0.077 |
| 22313233-R0 | 0.100 | | 0.158 | | 0.080 |
| 22313233-R1 | 0.055 | | 0.023 | | 0.158 |
| 22313233-R2 | 0.076 | 0.245 | 0.144 | 0.062 | 0.079 |
| 22313233-R3 | 0.028 | | 0.005 | | 0.036 |
| 23132233-R0 | 0.056 | | | | 0.013 |
| 23132233-R1 | 0.050 | | 0.109 | | 0.045 |
| 23132233-R2 | 0.042 | | 0.009 | 0.178 | 0.076 |
| 23132233-R3 | 0.061 | | 0.052 | | 0.028 |
| 32312231-R0 | 0.119 | 0.119 | 0.019 | | 0.085 |
| 32312231-R1 | 0.114 | 0.046 | 0.133 | | 0.108 |
| 32312231-R2 | 0.088 | 0.061 | 0.062 | 0.146 | 0.107 |
| 32312231-R3 | 0.036 | | 0.014 | | 0.031 |
| 32312333-R0 | 0.081 | 0.074 | 0.089 | 0.034 | 0.071 |
| 32312333-R1 | 0.068 | 0.111 | 0.045 | 0.020 | 0.056 |
| 32312333-R2 | 0.051 | 0.107 | 0.035 | 0.019 | 0.049 |
| 32312333-R3 | 0.107 | 0.070 | 0.079 | 0.133 | 0.030 |
| 32313233-R0 | 0.090 | 0.149 | 0.049 | 0.120 | 0.031 |
| 32313233-R1 | 0.143 | 0.105 | 0.036 | 0.011 | 0.063 |
| 32313233-R2 | 0.064 | 0.053 | 0.033 | 0.020 | 0.083 |
| 32313233-R3 | 0.064 | 0.093 | 0.073 | 0.034 | 0.013 |

| | diphenyl ether | 2-amino-5-chloro-benzoxazole | propranolol | chloroxazone | tolbutamide | 12-pNCA |
|---|---|---|---|---|---|---|
| 11111111-R0 | 0.735 | | 0.162 | 0.148 | 0.096 | 0.052 |
| 11111111-R1 | 0.118 | 0.364 | 0.054 | 0.128 | 0.106 | 0.076 |
| 11111111-R2 | 0.030 | 0.112 | 0.113 | 0.120 | 0.067 | 0.159 |
| 11111111-R3 | 0.066 | 0.189 | 0.092 | 0.082 | 0.118 | 0.083 |
| 22222222-R0 | 0.264 | | 0.261 | 0.005 | 0.159 | 0.125 |
| 22222222-R1 | 0.119 | 0.255 | 0.076 | 0.144 | 0.144 | 0.040 |
| 22222222-R2 | 0.081 | 0.251 | 0.085 | 0.108 | 0.099 | 0.011 |
| 22222222-R3 | 0.070 | 0.058 | 0.155 | 0.123 | 0.086 | 0.096 |
| 33333333-R3 | 0.094 | 0.082 | 0.110 | 0.155 | 0.088 | 0.068 |
| 11113311-R0 | 0.370 | | 0.117 | 0.083 | 0.000 | 0.058 |
| 11113311-R1 | 0.032 | 0.622 | 0.084 | 0.127 | 0.079 | 0.007 |
| 11113311-R2 | 0.079 | 0.177 | 0.130 | 0.102 | 0.038 | 0.012 |
| 11113311-R3 | 0.065 | 0.110 | 0.110 | 0.176 | 0.022 | 0.102 |
| 12112333-R0 | 0.034 | | 0.193 | 0.114 | 0.067 | 0.073 |
| 12112333-R1 | 0.104 | 0.065 | 0.177 | 0.137 | 0.069 | 0.075 |
| 12112333-R2 | 0.081 | 0.115 | 0.160 | 0.019 | 0.073 | 0.129 |
| 12112333-R3 | 0.035 | 0.064 | 0.082 | 0.066 | 0.115 | 0.133 |
| 21113312-R0 | 0.176 | | 0.156 | 0.053 | 0.156 | 0.118 |
| 21113312-R1 | 0.046 | 0.075 | 0.156 | 0.051 | 0.058 | 0.250 |
| 21113312-R2 | 0.182 | 0.183 | 0.182 | 0.088 | 0.051 | 0.379 |
| 21113312-R3 | 0.044 | 0.005 | 0.350 | 0.121 | 0.110 | 0.080 |
| 21313111-R0 | 0.092 | | 0.138 | 0.167 | 0.107 | |
| 21313111-R1 | 0.032 | 0.239 | 0.135 | 0.107 | 0.083 | 0.095 |
| 21313111-R2 | 0.069 | 0.424 | 0.083 | 0.106 | 0.088 | |
| 21313111-R3 | 0.107 | 0.195 | 0.035 | 0.145 | 0.127 | |
| 21313311-R0 | 0.078 | | 0.041 | 0.168 | 0.105 | |
| 21313311-R1 | 0.024 | 0.448 | 0.029 | 0.097 | 0.072 | |
| 21313311-R2 | 0.049 | 0.020 | 0.183 | 0.084 | 0.049 | |
| 21313311-R3 | 0.111 | 0.131 | 0.148 | 0.091 | 0.040 | |
| 21333233-R0 | 0.188 | 0.377 | 0.159 | 0.133 | 0.128 | |
| 21333233-R1 | 0.192 | 0.189 | 0.085 | 0.074 | 0.120 | |
| 21333233-R2 | 0.044 | 0.026 | 0.119 | 0.117 | 0.062 | 0.105 |
| 21333233-R3 | 0.182 | 0.067 | 0.043 | 0.082 | 0.041 | |
| 22132231-R0 | 0.398 | | 0.677 | 0.060 | 0.189 | |
| 22132231-R1 | 0.077 | | 0.183 | 0.166 | 0.110 | |
| 22132231-R2 | 0.092 | | 0.063 | 0.148 | 0.073 | |
| 22132231-R3 | 0.014 | 0.137 | 0.142 | 0.160 | 0.044 | |
| 22213132-R0 | 0.147 | 0.156 | 0.166 | 0.073 | 0.137 | |
| 22213132-R1 | 0.058 | 0.339 | 0.098 | 0.147 | 0.030 | 0.048 |
| 22213132-R2 | 0.039 | 0.070 | 0.124 | 0.120 | 0.005 | |
| 22213132-R3 | 0.052 | 0.119 | 0.144 | 0.111 | 0.114 | 0.000 |
| 22312333-R0 | 0.367 | | 0.151 | 0.132 | 0.170 | |
| 22312333-R1 | 0.068 | 0.266 | 0.098 | 0.085 | 0.076 | |
| 22312333-R2 | 0.059 | 0.042 | 0.150 | 0.091 | 0.016 | |
| 22312333-R3 | 0.127 | 0.153 | 0.121 | 0.264 | 0.038 | |
| 22313233-R0 | 0.134 | | 0.334 | 0.246 | 0.127 | |
| 22313233-R1 | 0.034 | 0.154 | 0.101 | 0.079 | 0.104 | |
| 22313233-R2 | 0.019 | 0.110 | 0.006 | 0.134 | 0.106 | |
| 22313233-R3 | 0.141 | 0.155 | 0.040 | 0.081 | 0.104 | |
| 23132233-R0 | 0.095 | 0.058 | 0.092 | 0.182 | 0.086 | |
| 23132233-R1 | 0.050 | 0.060 | 0.012 | 0.116 | 0.078 | |
| 23132233-R2 | 0.067 | 0.078 | 0.122 | 0.091 | 0.118 | |
| 23132233-R3 | 0.047 | 0.146 | 0.053 | 0.089 | 0.098 | |
| 32312231-R0 | 0.034 | | 0.167 | 0.105 | 0.177 | |
| 32312231-R1 | 0.074 | 0.531 | 0.050 | 0.102 | 0.054 | 0.190 |
| 32312231-R2 | 0.058 | 0.174 | 0.096 | 0.191 | 0.088 | 0.085 |
| 32312231-R3 | 0.118 | 0.054 | 0.055 | 0.117 | 0.051 | |
| 32312333-R0 | 0.015 | 0.056 | 0.137 | 0.077 | 0.125 | |
| 32312333-R1 | 0.113 | 0.014 | 0.052 | 0.102 | 0.042 | 0.457 |
| 32312333-R2 | 0.097 | 0.150 | 0.173 | 0.023 | 0.068 | 0.139 |
| 32312333-R3 | 0.075 | 0.095 | 0.050 | 0.078 | 0.069 | |
| 32313233-R0 | 0.140 | 0.050 | 1.863 | 0.074 | 0.067 | |
| 32313233-R1 | 0.089 | 0.184 | 0.147 | 0.078 | 0.044 | 0.062 |
| 32313233-R2 | 0.113 | 0.102 | 0.122 | 0.072 | 0.035 | 0.346 |
| 32313233-R3 | 0.034 | 0.005 | 0.132 | 0.133 | 0.039 | |

TABLE 6
Summary of error statistics for collected
absorbance data sorted by substrates. The percent of the standard
deviation divided by the average value and the percentage of data
points retained for the analysis are measures of data quality. For
each substrate, 65 data points were collected. The
Triplicates/Duplicates column indicates how many of those data
points were used for the analysis performed here.

| | % SD/ | % | |
|---|---|---|---|
| | avg | points | Triplicates/ |
| Substrate | (mean) | retained | Duplicates |
| 2-phenoxyethanol (PE) | 7.1 | 99 | 63/2 |
| ethoxybenzene (EB) | 10.2 | 87 | 39/26 |
| ethyl phenoxyacetate (PA) | 8.5 | 95 | 56/9 |
| 3-phenoxytoluene (PT) | 8.0 | 94 | 53/12 |
| ethyl 4-phenylbutyrate (PB) | 6.7 | 100 | 65/0 |
| diphenyl ether (DP) | 10.9 | 95 | 56/9 |
| zoxazolamine (ZX) | 16.0 | 87 | 40/25 |
| propranolol (PR) | 15.6 | 90 | 45/20 |
| chlorzoxazone (CH) | 11.2 | 99 | 63/2 |
| tolbutamide (TB) | 8.5 | 99 | 63/2 |
| 12-p-nitrophenoxycarboxylic acid (PN) | 11.8 | 87 | 40/25 |

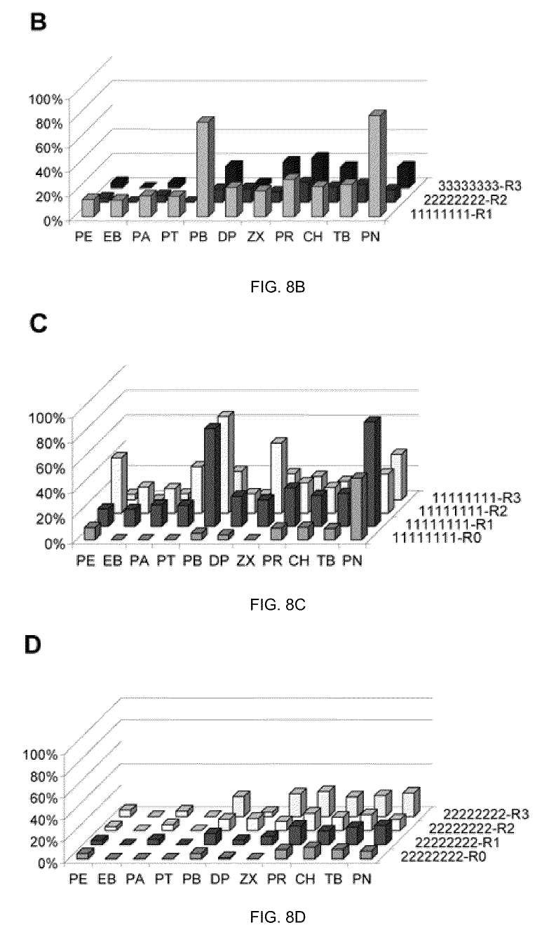

The data compare the chimeras with respect to their activities on a given substrate and also to compare their activity profiles and therefore their specificities. Chimeras having a similar profile form the same relative amounts of products from all substrates and are therefore likely to have similar specificities. To better visualize differences among chimeras, the highest average absorbance value for a given substrate was set to 100%, and all other absorbances for the same substrate, but different chimeras, were normalized to this. FIG. 8 shows the substrate-activity profiles in the form of bar plots.

FIG. 8A shows the normalized substrate-activity profiles of the A1 and A2 peroxygenases. Both have relatively low or no activity on any of the substrates except PN, where A1 makes about an order of magnitude more product than does A2. Profiles for the reconstituted parent holoenzymes are shown in FIG. 8B. Fusion of A1 and R1 generated an enzyme with profile peaks on ethyl 4-phenylbutyrate (PB) and PN. A1 is in fact the second-best-performing enzyme on PB. The A1 peroxygenase activity on this substrate, however, is among the worst, showing that peroxygenase specificity does not necessarily predict that of the monooxygenase. Fusion of A2 to R2 slightly increased activity relative to A2, but did not alter the profile. The A3-R3 holoenzyme exhibits some activity on the drug-like substrates (PR, TB, CH) as well as PN and PB.

Fusion of the A1 and A2 heme domains to other reductase domains yields holoenzymes that are active on some substrates (FIGS. 8C and 8D). The A2 fusions have relatively low activities. A1 fusions with R1 and R2, on the other hand, created highly active enzymes with different specificities: the A1-R1 profile has peaks on PN and PB, while that of A1-R2 has peaks on PB, phenoxyethanol (PE) and zoxazolamine (ZX). The A1-R3 fusion is less active on nearly all substrates.

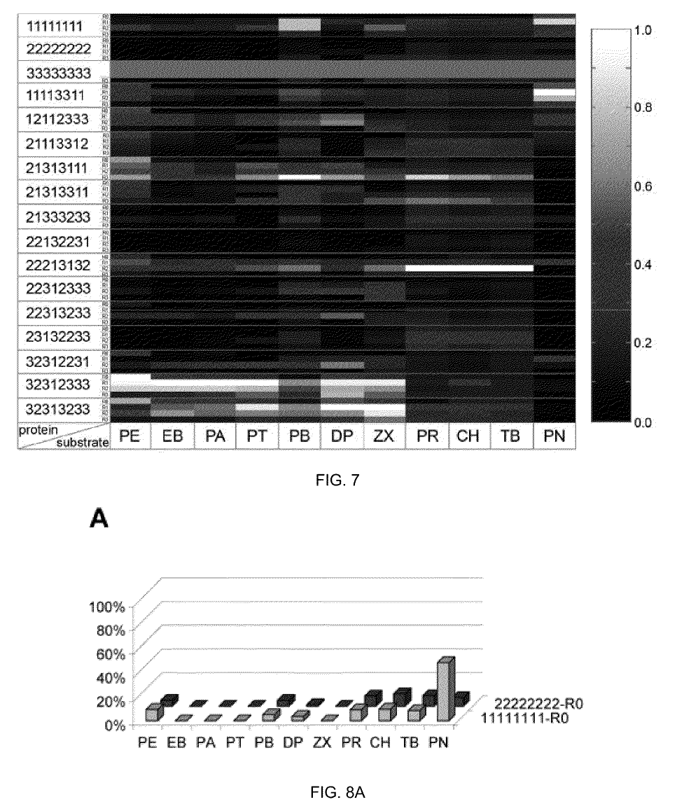

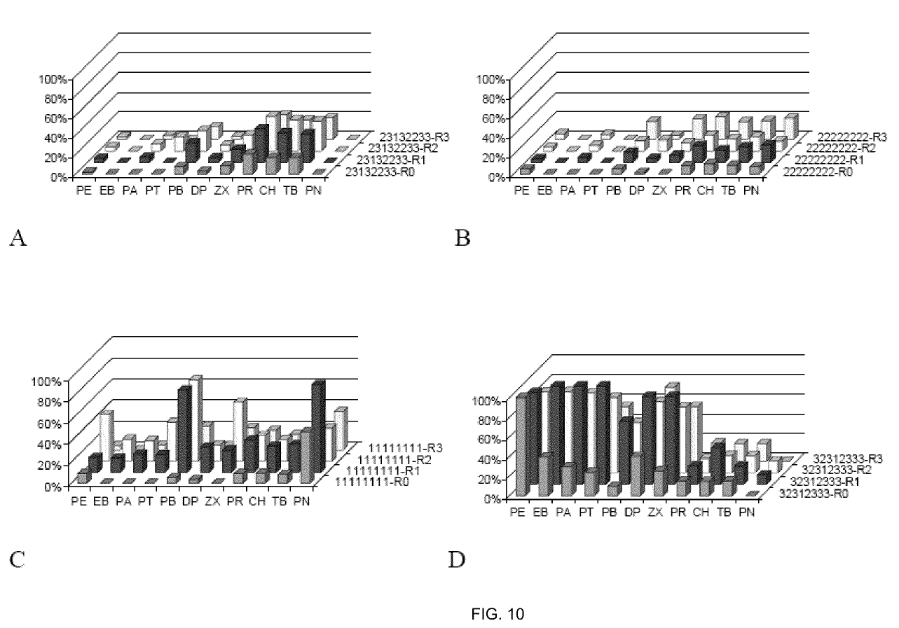

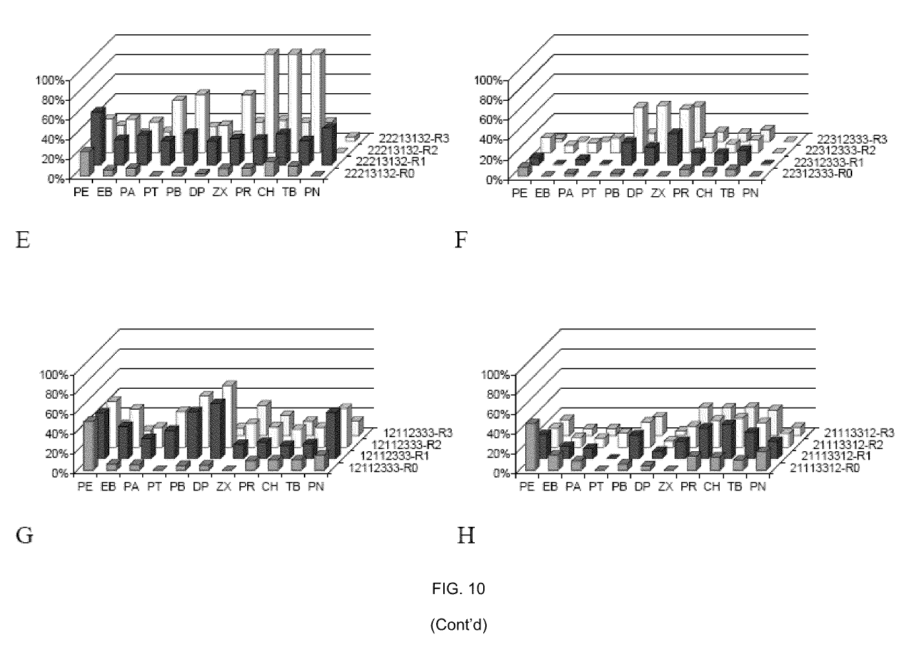

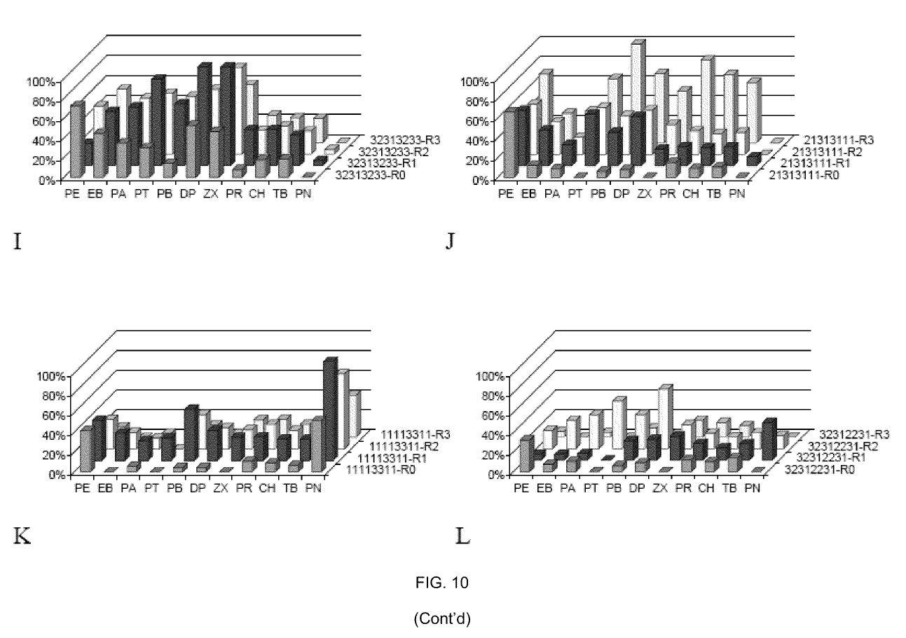

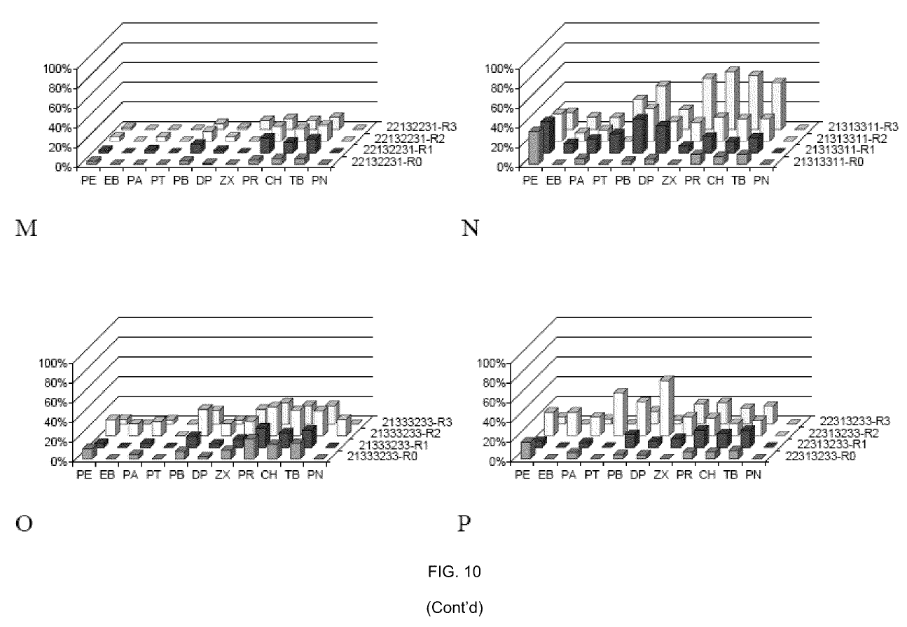

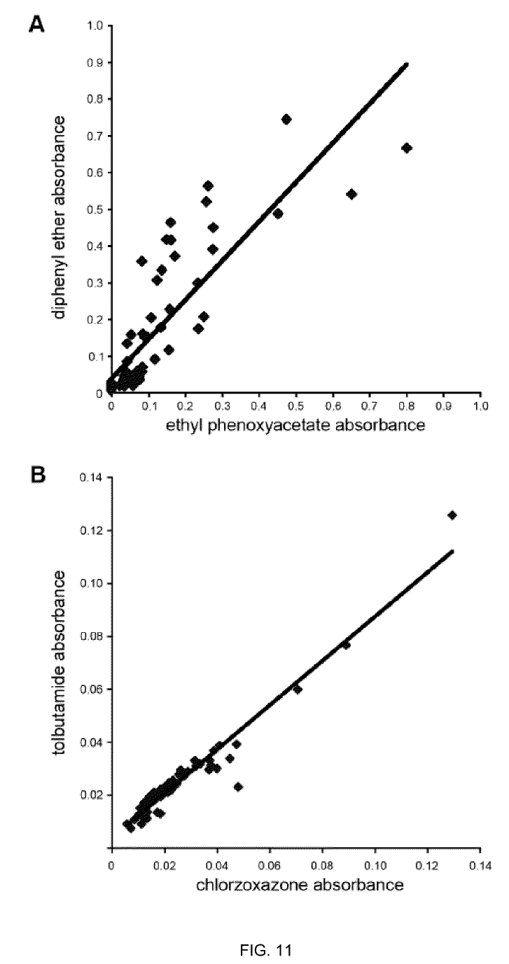

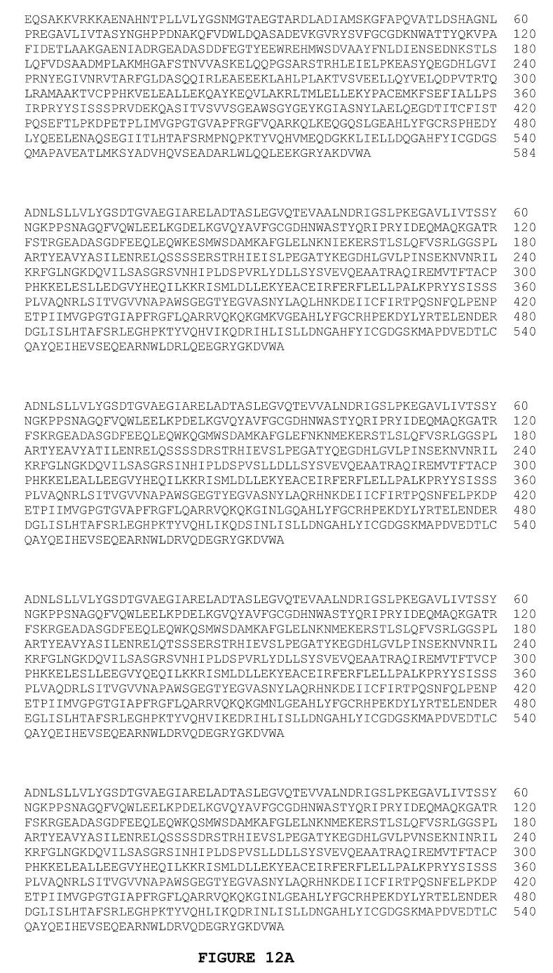

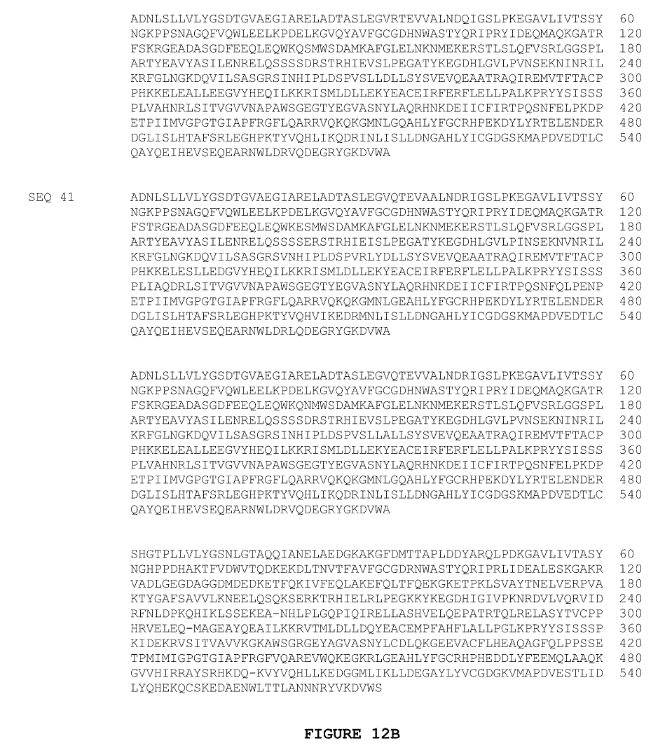

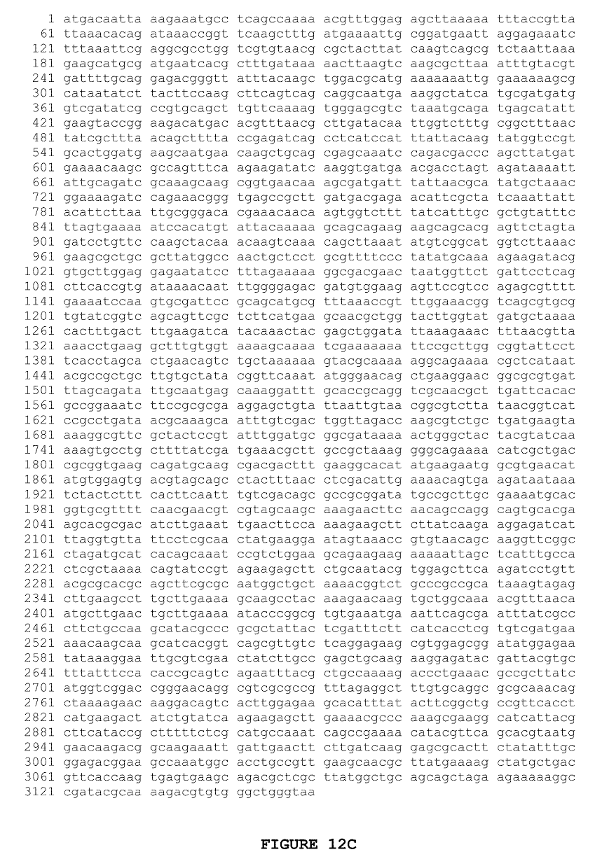

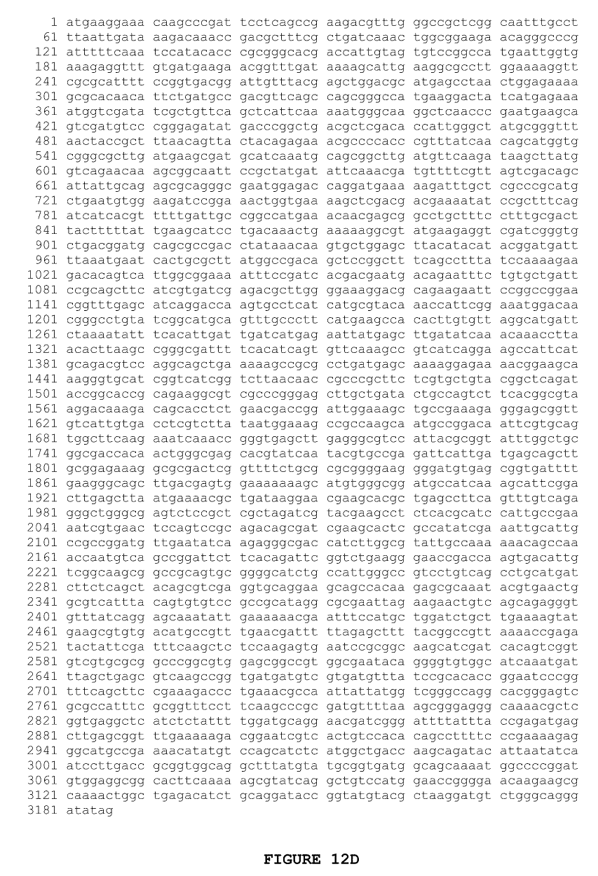

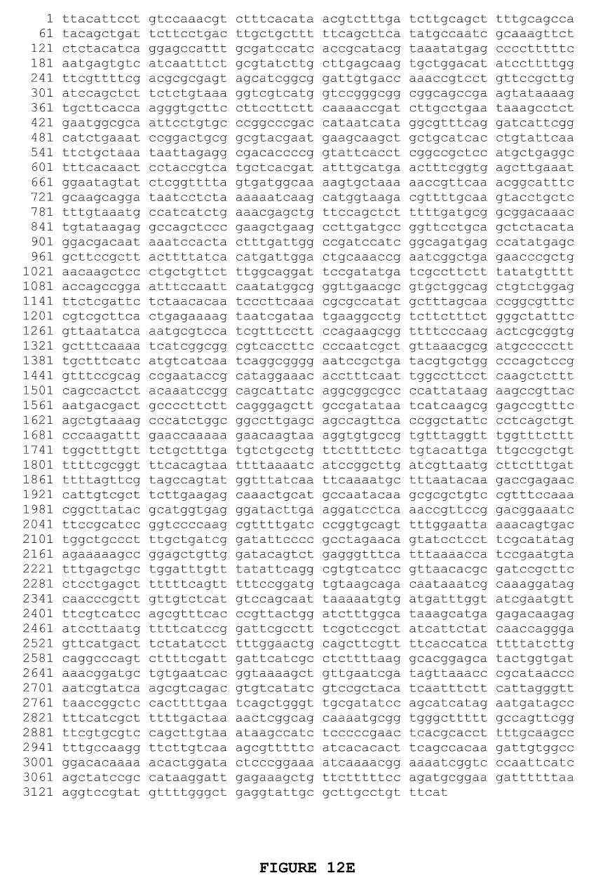

The 14 chimeric heme domains generated 56 chimeric peroxygenases and monooxygenases. Nearly all the chimera fusions outperformed even the best parent holoenzyme, and chimeric peroxygenases consistently outperformed the parent peroxygenases (FIG. 7 and FIG. 10). The best enzyme for each substrate is listed in Table 7. All the best enzymes are chimeras. Most of the best enzymes are also holoenzymes-only PE has a peroxygenase as the best catalyst.

TABLE 7
Summary of most active chimeric proteins for each substrate. Pairwise
correlation matrix of the activities on all substrates. R² values are reported.
Bold and underlined = 0.7-1.0; Underlined = 0.4-0.7; Regular = 0.0-0.4.

| Protein | | PE | EB | PA | PT | PB | DP | ZX | PR | CH | TB | PN |
|---|---|---|---|---|---|---|---|---|---|---|---|---|
| 32312231-R0 | PE | N.A. | 0.61 | 0.48 | 0.37 | 0.18 | 0.35 | 0.15 | 0.01 | 0.05 | 0.02 | 0.01 |
| 32312231-R1 | EB | | N.A. | 0.92 | 0.80 | 0.41 | 0.73 | 0.56 | 0.04 | 0.13 | 0.06 | 0.00 |
| 32312231-R1 | PA | | | N.A. | 0.81 | 0.39 | 0.71 | 0.62 | 0.04 | 0.14 | 0.06 | 0.00 |
| 32312231-R1 | PT | | | | N.A. | 0.56 | 0.85 | 0.66 | 0.14 | 0.24 | 0.16 | 0.00 |
| 21313111-R3 | PB | | | | | N.A. | 0.49 | 0.49 | 0.36 | 0.37 | 0.33 | 0.08 |
| 32313233-R1 | DP | | | | | | N.A. | 0.58 | 0.05 | 0.10 | 0.06 | 0.00 |
| 32313233-R1 | ZX | | | | | | | N.A. | 0.18 | 0.29 | 0.21 | 0.00 |
| 22213132-R2 | PR | | | | | | | | N.A. | 0.91 | 0.95 | 0.00 |
| 22213132-R2 | CH | | | | | | | | | N.A. | 0.94 | 0.00 |
| 22213132-R2 | TB | | | | | | | | | | N.A. | 0.00 |
| 11113311-R1 | PN | | | | | | | | | | | N.A. |

The data show that there exists a discrete set of characteristic substrate-activity profiles to which each chimera can be uniquely assigned. A k-means clustering analysis was applied to the normalized absorbance data to better understand the functional diversity. K-means clustering, a statistical algorithm that partitions data into clusters based on data similarity, mutants exhibiting similar substrate specificities and protein fragments (4-7 residues) of similar structure and interacting nucleotide pairs with similar 3D structures. For this analysis, the normalized data were used to ensure that each of the 11 dimensions is given equal weight by the clustering algorithm. The clustering was performed over values of k (number of clusters) ranging from k=2 to k=8. The highest silhouette value was observed at k=5.

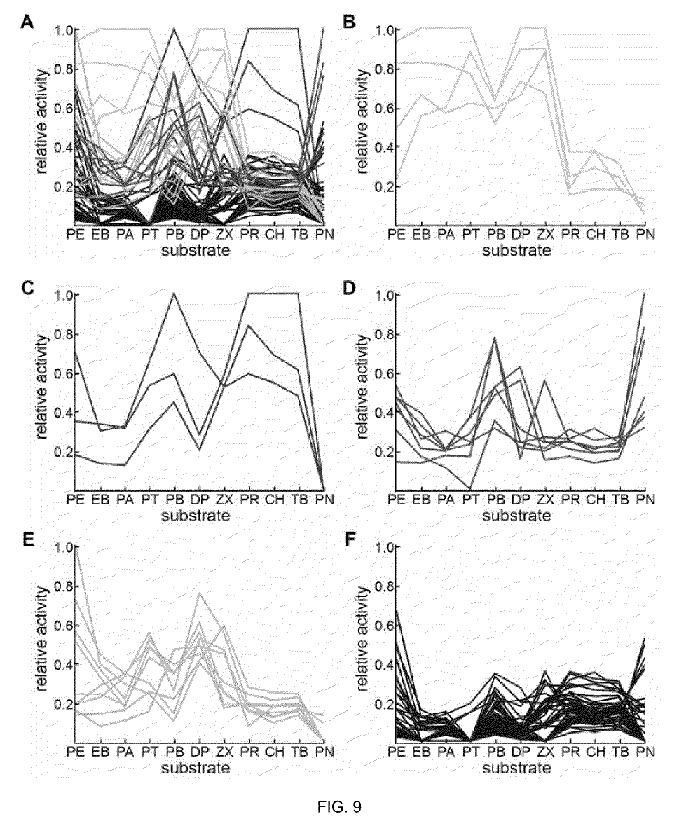

The cluster composition for k=5 is depicted in FIG. 9. Cluster 1, consisting of chimeras 32312333-R1/R2 and 32313233-R1/R2 (FIG. 9B), is characterized by low relative activities on CH, TB, PR and PN and high relative activities on all other substrates. In fact, two of these chimeras are the best enzymes on all the remaining substrates except PB and PE.

Cluster 2 is made up of 22213132-R2, 21313111-R3, 21313311-R3, which are the most active enzymes on TB, CH and PR (FIG. 9C). Cluster 2 enzymes are entirely inactive on PN and show low activity on most of the substrates that cluster 1 enzymes accept (PE, DP, PA and EB). Relative activities on the remaining substrates (i.e. PB, ZX and PT) are moderate (although lower than cluster 1 chimeras). An exception is 21313111-R3, which is the best enzyme for PB and also fairly good on PE and DP.

Cluster 3 contains chimeras A1-R1/R2, 12112333-R1/R2, 11113311-R1/R2 and 22213132-R1 (FIG. 9D). The A1-like sequences are characterized by high relative activity on PN (on which 11113311-R1/R2 and A1-R1 are the three top-ranking enzymes), and moderate to high relative activity on PB and moderate activity on PE.

Cluster 4 contains 21313111-R1/R2, 22313233-R2, 22312333-R2, 32312231-R2, 32312333-R0, 32312333-R3, 32313233-R0, and 32313233-R3 (FIG. 9E). This cluster is characterized by having the highest relative activity on PE, in addition to moderate activities on PT, DP and ZX. The remaining chimeras appear in a fifth cluster with relatively low activity on everything except PN and PE (FIG. 9F). This cluster contains parental sequences A1-R0, A1-R3, A2-R0, A2-R1/R2/R3 and A3-R3. Native sequences are thus found in two of the clusters. The remaining clusters (1, 2 and 4) are made up of highly active chimeras that have acquired novel profiles.

The partition created by a clustering algorithm shows that the presence and identity of the reductase can alter the activity profile and thus the specificity of a heme domain sequence. For example, the R1 and R2 fusions of 32312333 and 32313233 appear in cluster 1, whereas their R0 and R3 counterparts are in cluster 4. Sequences 22213132 and 21313111 also behave differently when fused to different reductases. 22213132-R2, for example, displays pronounced peaks on substrates TB, CH and PR that are not present in the corresponding peroxygenase and R1/R3 profiles (FIG. 10E) and is thus the only member with this heme domain sequence appearing in cluster 2. 21313111-R3 and 21313111-R2/R1 have nearly opposite profiles (FIG. 10J) and consequently appear in different clusters. Thus the best choice of reductase depends on both the substrate and the chimera sequence.

The observed correspondence between the three substrate groups and chimera clusters 1, 2 and 3 illustrates that each group can be associated with a cluster made up of or containing the top-performing enzymes for the substrates in that group. Some degree of correspondence can be expected, given how the partitions were constructed. However, because intra-group correlations are not one and inter-group correlations are not zero, the correspondence is not perfect. For this reason there exist chimeras whose profiles exhibit peaks on only certain members of a group (cluster 4) and others that exhibit peaks on members of different groups (cluster 2 and 3 chimeras). Cluster 4 chimeras have peaks on only certain members of group A and are thus responsible for the lower correlations among group A substrates. Some cluster 2 and cluster 3 chimeras exhibit peaks on PB (on the edge of group A) as well as group B and C, respectively. In fact although PB correlates mostly with group A core substrates it shares its top-performing enzymes with groups B and C and thus displays a hybrid behavior. This is why PB correlates less with group A than core substrates do and why it has higher correlations with group B and C members than any other substrate not belonging to these groups.

Because chimeras displaying high relative activity have more weight in determining the correlation coefficients, the top enzymes for one member of a substrate group will usually be among the top ones for all members of that group. The clearer the definition of the substrate groups, the more likely this is to hold. Given the many important applications of P450s in medicine and biocatalysis, and the lack of high-throughput screens for many compounds of interest, an approach to screening that is based on carefully chosen ‘surrogate’ substrates could significantly enhance our ability to identify useful catalysts. Clearly, any member of a well-defined substrate group can be a surrogate for other members of that group. Further analysis may also help to identify the critical physical, structural or chemical properties of substrates belonging to a known group. This will make it possible to predict which chimeras will be most active on a new, untested substrate.

Substrate specificity of heme-reductase fusion polypeptides and comparison to heme domain perooxygenase activity: Chimeric heme domains were fused to each of the three wildtype reductase domains after amino acid residue 463 when the last block originates from CYP102A1 and 466 for CYP102A2 and CYP102A3. The holoenzymes were constructed by overlap extension PCR and/or ligation and cloned into the pCWori expression vector. All constructs were confirmed by sequencing. Table 8 provides exemplary sequences associated with the chimeras described herein.

TABLE 8

| | | |
|---|---|---|
| Position | Parent | Sequence (amino acid) |

| | | |
|---|---|---|
| 1 | A1 | TIKEMPQPKTFGELKNLPLLNTDKPVQALMKIADEL |
| | | GEIFKFEAPGRVTRYLSSQRLIKFACDE |
| | | (SEQ ID NO:4) |
| 1 | A2 | KETSPIPQPKTFGPLGNLPLIDKDKPTLSLIKLAEE |
| | | QGPIFQIHTPAGTTIVVSGHELVKEVCDE |
| | | (SEQ ID NO:5) |
| 1 | A3 | KQASAIPQPKTYGPLKNLPHLEKEQLSQSLWRIADE |
| | | LGPIFRFDFPGVSSVFVSGHNLVAEVCDE |
| | | (SEQ ID NO:6) |
| 2 | A1 | SRFDKNLSQALKFVRDFAGDGLATSWTHEKNWKKAH |
| | | NILLPSFSQQAMKGYHAMMVDI |
| | | (SEQ ID NO:7) |
| 2 | A2 | ERFDKSIEGALEKVRAFSGDGLATSWTHEPNWRKAH |
| | | NILMPTFSQRAMKDYHEKMVDI |
| | | (SEQ ID NO:8) |
| 2 | A3 | KRFDKNLGKGLQKVREFGGDGLATSWTHEPNWQKAH |
| | | RILLPSFSQKAMKGYHSMMLDI |
| | | (SEQ ID NO:9) |
| 3 | A1 | AVQLVQKWERLNADEHIEVPEDMTRLTLDTIGLCGF |
| | | NYRFNSFY |
| | | (SEQ ID NO:10) |
| 3 | A2 | AVQLIQKWARLNPNEAVDVPGDMTRLTLDTIGLCGF |
| | | NYRFNSYY |
| | | (SEQ ID NO:11) |
| 3 | A3 | ATQLIQKWSRLNPNEEIDVADDMTRLTLDTIGLCGF |
| | | NYRFNSFY |
| | | (SEQ ID NO:12) |
| 4 | A1 | RDQPHPFITSMVRALDEAMNKLQRANPDDPAYDENK |
| | | RQFQEDIKVMNDLV |
| | | (SEQ ID NO:13) |
| 4 | A2 | RETPHPFINSMVRALDEAMHQMQRLDVQDKLMVRTK |
| | | RQFRYDIQTMFSLV |
| | | (SEQ ID NO:14) |
| 4 | A3 | RDSQHPFITSMLRALKEAMNQSKRLGLQDKMMVKTK |
| | | LQFQKDIEVMNSLV |
| | | (SEQ ID NO:15) |
| 5 | A1 | DKIIADRKASGEQ, SDDLLTHMLNGKDPETGEPLD |
| | | DENIRYQIITFLIAGHET |
| | | (SEQ ID NO:16) |
| 5 | A2 | DSIIAERRANGDQDEKDLLARMLNVEDPETGEKLDD |
| | | ENIRFQIITFLIAGHET |
| | | (SEQ ID NO:17) |
| 5 | A3 | DRMIAERKANPDENIKDLLSLMLYAKDPVTGETLDD |
| | | ENIRYQIITFLIAGHET |
| | | (SEQ ID NO:18) |
| 6 | A1 | TSGLLSFALYFLVKNPHVLQKAAEEAARVLVDPVPS |
| | | YKQVKQLKYVGMVLNEALRLWPTAA |
| | | (SEQ ID NO:19) |
| 6 | A2 | TSGLLSFATYFLLKHPDKLKKAYEEVDRVLTDAAPT |
| | | YKQVLELTYIRMILNESLRLWPTA |
| | | (SEQ ID NO:20) |
| 6 | A3 | TSGLLSFAIYCLLTHPEKLKKAQEEADRVLTDDTPE |
| | | YKQIQQLKYIRMVLNETLRLYPTA |
| | | (SEQ ID NO:21) |
| 7 | A1 | PAFSLYAKEDTVLGGEYPLEKGDELMVLIPQLHRDK |
| | | TIWGDDVEEFRPERFENPSAIPQHAFKPFGNGQRAC |
| | | IGQQ |
| | | (SEQ ID NO:22) |
| 7 | A2 | PAFSLYPKEDTVIGGKFPITTNDRISVLIPQLHRDR |
| | | DAWGKDAEEFRPERFEHQDQVPHHAYKPFGNGQRAC |
| | | ICMQ |
| | | (SEQ ID NO:23) |
| 7 | A3 | PAFSLYAKEDTVLGGEYPISKGQOVTVLIPKLHRDQ |
| | | NAWGPDAEDFRPERFEDPSSIPHHAYKPFGNGQRAC |
| | | IGMQ |
| | | (SEQ ID NO:24) |
| 8 | A1 | FALHEATLVLGMMLKHFDFEDHTNYELDIKETLTLK |
| | | PEGFVVKAKSKKIPLGGIPSPST |
| | | (SEQ ID NO:25) |
| 8 | A2 | FALHEATLVLGMILKYFTLIDHENYELDIKQTLTLK |
| | | PGDFHISVQSRHQEAIHADVQAAE |
| | | (SEQ ID NO:26) |
| 8 | A3 | FALQEATMVLGLVLKHFELINHTGYELKIKEALTIK |
| | | PDDFKITVKPRKTAAINVQRKEQA |
| | | (SEQ ID NO:27) |

Proteins were expressed in E. coli and purified by anion exchange on Toyopearl SuperQ-650M from Tosoh. After binding of the proteins, the matrix was washed with a 30 mM NaCl buffer, and proteins were eluted with 150 mM NaCl (all buffers used for purification contained 25 mM phosphate buffer pH 8.0). Proteins were rebuffered into 100 mM phosphate buffer and concentrated using 30,000 MWCO Amicon Ultra centrifugal filter devices (Millipore). Proteins were stored at −20° C. in 50% glycerol.

Protein concentration was measured by CO absorption at 450 nm. A protein concentration of 1 μM was chosen for the activity assays. Protein concentrations were re-assayed in 96-well format and determined to be 0.88 μM+/−13% (SD/average).

Proteins were assayed for mono- or peroxygenase activities in 96-well plates. Heme domains were assayed for peroxygenase activity using hydrogen peroxide as the oxygen and electron source. Reductase domain fusion proteins were assayed for monooxygenase activity, using molecular oxygen and NADPH. Reactions were carried out in 100 mM EPPS buffer pH 8, 1% acetone, 1% DMSO, 1 μM protein in 120 μl volumes. Substrate concentrations depended on their solubility under the assay conditions. Final concentrations were: 2-phenoxyethanol (PE), 100 mM; ethoxybenzene (EB), 50 mM; ethyl phenoxyacetate (PA), 10 mM; 3-phenoxytoluene (PT), 10 mM; ethyl 4-phenylbutyrate (PB), 5 mM; diphenyl ether (DP), 10 mM; zoxazolamine (ZX), 5 mM; propranolol (PR), 4 mM; chlorzoxazone (CH), 5 mM; tolbutamide (TB), 10 mM; 12-p-nitrophenoxycarboxylic acid (PN), 0.25 mM. The reaction was initiated by the addition of NADPH or hydrogen peroxide stock solution (final concentration of 500 μM NADPH or 2 mM hydrogen peroxide) and mixed briefly. After 2 hrs at room temperature, reactions with substrates 1-10 were quenched with 120 μl of 0.1 M NaOH and 4 M urea. Thirty-six μl of 0.6% (w/v) 4-aminoantipyrine (4-AAP) was then added. The 96-well plate reader was zeroed at 500 nm and 36 μl of 0.6% (w/v) potassium persulfate was added. After 20 min, the absorbance at 500 nm was read. Reactions on PN were monitored directly at 410 nm by the absorption of accumulated 4-nitrophenol. All experiments were performed in triplicate, and the absorption data were averaged.

The background absorbance (BG) was subtracted from the raw data. BG reactions contained buffer, cofactor and substrate in the absence of protein sample and were done in triplicates. All absorbance measurements were done once on three separate samples (triplicate sampling). Data points with a SD/average≧20% that did not lie within the average±1.1*SD were eliminated. 1.1*SD was chosen so that for each substrate at least 85% of the points were retained. This never resulted in the elimination of more than one point from each triplicate set of measurements. All points with an average absorbance<BG were set to zero, because they are assumed to belong to inactive proteins.

K-means clustering is a partitioning method that divides a set of observations into k mutually exclusive clusters. K-means treats each data point as an object having a location in m-dimensional space (m=11 in this analysis) [23]. It then finds a partition such that members of the same cluster are as close as possible to each other and as far as possible to members of other clusters. For this reason, a measure of the meaningfulness of a partition is given by the silhouette value

$$s = avg ( \frac{b ( i ) - a ( i )}{max [ a ( i ) , b ( i ) ]} ) ,$$

where a(i) is the average distance of point i to all other points in its cluster and b(i) is the average distance of point i to all points in the closest cluster. It is evident that −1≦s≦1 and the quality of the clustering increases as s->1. Distances are measured by the square of the Euclidean distance.

Table 9 below demonstrates chimeric heme domains having peroxygenase activity. Table 10 demonstrates 40 holoenzymes, which are fusion of chimeric heme domains of the disclosure and a various reductase domains. The holoenzymes of Table 10 function as monooxygenases and exhibit novel activities, not exhibited by the parental (i.e., wild-type) proteins. Activities of the holoenzymes were tested on 12-para-nitrophenoxydodecanoic acid (S1), ethoxybenzene (S2), ethyl phenoxyacetate (S3), 3-phenoxyttoluene (S4), ethyl 4-pehylbutyrate (S5), diphenyl ether (S6), propranolol (S7), chlorzoxazone (S8) and tolbutamide (S9). Final substrate concentrations were: 2-phenoxyehtanol, 10 mM; ethoxybenzene, 25 mM; ethyl phenoxyacetate, 10 mM; 3-phenoxytoluene, 10 mM; ethyl 4-phenylbutyrate, 5 mM; diphenyl ether, 10 mM; propranolol, 2 mM; chlorzoxazone, 5 mM; tolbutamide, 10 mM; 12-p-nitrophenoxycarboxylic acid (12pNCA), 0.5 mM. After 2 hours at room temperature, reactions (except 12pNCA) were quenched with 120 μl of 0.1 M NaOH and 4 M urea. Thirty-six μl of 0.6% (w/v) 4-aminoantipyrine (4-AAP) was then added. A 96-well plate reader was zeroed at 500 nm and 36 μl of 0.6% (w/v) potassium persulfate was added. After 20 minutes, the absorbance at 500 nm was read. Reactions on 12PNCA were monitored directly at 410 nm by the absorption of accumulated 4-nitrophenol.

TABLE 9
Average peroxygenase activities (in absorbance
units) and standard deviations (based on three parallel
measurements) of stable cytochrome P450 chimeras on 9 substrates.

| | S1 | | S2 | | S3 | | S4 | | S5 | |
|---|---|---|---|---|---|---|---|---|---|---|
| sequence | Activity | Std | Activity | Std | Activity | Std | Activity | Std | Activity | Std |
| 21311231 | 0.116 | 0.024 | 0.0380 | 0.0016 | 0.0369 | 0.0152 | 0.0364 | 0.0056 | −0.0084 | 0.0703 |
| 21311233 | 0.128 | 0.048 | 0.1225 | 0.0126 | 0.1756 | 0.0127 | 0.1223 | 0.0109 | 0.0978 | 0.0008 |
| 21312133 | 0.278 | 0.038 | 0.1117 | 0.0044 | 0.1470 | 0.0125 | 0.0988 | 0.0184 | 0.1003 | 0.0035 |
| 21312231 | 0.178 | 0.116 | 0.0686 | 0.0081 | 0.0837 | 0.0029 | 0.0725 | 0.0035 | 0.0577 | 0.0016 |
| 21312311 | 0.257 | 0.204 | 0.0768 | 0.0013 | 0.1231 | 0.0050 | 0.0973 | 0.0024 | 0.0697 | 0.0022 |
| 21312332 | 0.173 | 0.168 | 0.1160 | 0.0110 | 0.1066 | 0.0085 | 0.0974 | 0.0112 | 0.0931 | 0.0085 |
| 21313233 | 0.298 | 0.172 | 0.0817 | 0.0021 | 0.1136 | 0.0097 | 0.0729 | 0.0019 | 0.0731 | 0.0057 |
| 21313331 | 0.559 | 0.441 | 0.0794 | 0.0024 | 0.1380 | 0.0092 | 0.0797 | 0.0037 | 0.0640 | 0.0031 |
| 21313333 | 0.165 | 0.042 | 0.0496 | 0.0053 | 0.0687 | 0.0394 | 0.0444 | 0.0017 | 0.0294 | 0.0251 |
| 22311233 | 0.186 | 0.090 | 0.1038 | 0.0042 | 0.1405 | 0.0114 | 0.1011 | 0.0021 | 0.0895 | 0.0048 |
| 22312233 | 0.185 | 0.026 | 0.1009 | 0.0023 | 0.1204 | 0.0040 | 0.0937 | 0.0092 | 0.0837 | 0.0073 |
| 22313331 | 0.206 | 0.006 | 0.1556 | 0.0162 | 0.2816 | 0.0150 | 0.1445 | 0.0188 | 0.1068 | 0.0037 |
| 22313231 | 0.211 | 0.093 | 0.1123 | 0.0097 | 0.2193 | 0.0123 | 0.0940 | 0.0044 | 0.0705 | 0.0020 |
| 22312331 | 0.353 | 0.160 | 0.0902 | 0.0052 | 0.1546 | 0.0146 | 0.0906 | 0.0034 | 0.0662 | 0.0058 |
| 21312331 | 0.195 | 0.029 | 0.0853 | 0.0008 | 0.1066 | 0.0035 | 0.0790 | 0.0082 | 0.0698 | 0.0042 |
| 21312313 | 0.202 | 0.101 | 0.1040 | 0.0061 | 0.1213 | 0.0033 | 0.1108 | 0.0048 | 0.0912 | 0.0060 |
| 22311333 | 0.109 | 0.044 | 0.0475 | 0.0024 | 0.0452 | 0.0339 | −0.0151 | 0.1341 | 0.0325 | 0.0300 |
| 22313333 | 0.237 | 0.061 | 0.1071 | 0.0037 | 0.2162 | 0.0034 | 0.1049 | 0.0034 | 0.0770 | 0.0062 |
| 21112333 | 0.280 | 0.206 | 0.0859 | 0.0073 | 0.1004 | 0.0043 | 0.0788 | 0.0049 | 0.0665 | 0.0032 |
| 21112233 | 0.227 | 0.130 | 0.0740 | 0.0035 | 0.0895 | 0.0039 | 0.0851 | 0.0223 | 0.0606 | 0.0027 |
| 21113333 | 0.122 | 0.021 | 0.2297 | 0.0045 | 0.2172 | 0.0115 | 0.2074 | 0.0160 | 0.1842 | 0.0127 |
| 21112331 | 0.295 | 0.091 | 0.0704 | 0.0030 | 0.0830 | 0.0030 | 0.0644 | 0.0017 | 0.0566 | 0.0030 |
| 22112233 | 0.105 | 0.062 | 0.1560 | 0.0118 | 0.1798 | 0.0029 | 0.1516 | 0.0193 | 0.1158 | 0.0039 |
| 21312213 | 0.324 | 0.030 | 0.1165 | 0.0070 | 0.2865 | 0.0176 | 0.0989 | 0.0067 | 0.0735 | 0.0020 |
| 21311333 | 0.140 | 0.072 | 0.0400 | 0.0044 | 0.0563 | 0.0118 | 0.0476 | 0.0070 | 0.0205 | 0.0275 |
| 21313313 | 0.235 | 0.069 | 0.0817 | 0.0037 | 0.0992 | 0.0085 | 0.0948 | 0.0077 | 0.0708 | 0.0023 |
| 22311331 | 0.205 | 0.012 | 0.0888 | 0.0061 | 0.1450 | 0.0039 | 0.0896 | 0.0213 | 0.0840 | 0.0019 |
| 21312211 | 0.235 | 0.022 | 0.1201 | 0.0104 | 0.2282 | 0.0126 | 0.1254 | 0.0164 | 0.0899 | 0.0091 |
| 21212233 | 0.227 | 0.130 | 0.0904 | 0.0043 | 0.1176 | 0.0046 | 0.0933 | 0.0082 | 0.0775 | 0.0039 |
| 22212333 | 0.150 | 0.027 | 0.1132 | 0.0052 | 0.1230 | 0.0075 | 0.1006 | 0.0145 | 0.0963 | 0.0067 |
| 21311311 | 0.300 | 0.067 | 0.0757 | 0.0028 | 0.1252 | 0.0099 | 0.0814 | 0.0065 | 0.0673 | 0.0050 |
| 21311313 | 0.162 | 0.050 | 0.1477 | 0.0083 | 0.1839 | 0.0142 | 0.1662 | 0.0139 | 0.1424 | 0.0097 |
| 21311331 | 0.119 | 0.072 | 0.0091 | 0.0426 | 0.0570 | 0.0471 | −0.3613 | 0.5680 | −0.1345 | 0.3222 |
| 21313231 | 0.159 | 0.051 | 0.1581 | 0.0264 | 0.1713 | 0.0195 | 0.1723 | 0.0120 | 0.1314 | 0.0181 |
| 22312333 | 0.141 | 0.058 | 0.1838 | 0.0143 | 0.1959 | 0.0066 | 0.1564 | 0.0387 | 0.1196 | 0.0102 |
| 22313233 | 0.151 | 0.018 | 0.0825 | 0.0032 | 0.1305 | 0.0134 | 0.0870 | 0.0031 | 0.0695 | 0.0018 |
| 21212333 | 0.239 | 0.101 | 0.1120 | 0.0050 | 0.1321 | 0.0062 | 0.1210 | 0.0025 | 0.0995 | 0.0014 |
| 21312333 | 0.171 | 0.021 | 0.1041 | 0.0040 | 0.1268 | 0.0077 | 0.1063 | 0.0030 | 0.0880 | 0.0031 |
| 11111111 | 0.296 | 0.033 | 0.0729 | 0.0018 | 0.0938 | 0.0118 | 0.0548 | 0.0018 | 0.0524 | 0.0033 |

| | S6 | | S7 | | S8 | | S9 | |
|---|---|---|---|---|---|---|---|---|
| sequence | Activity | Std | Activity | Std | Activity | Std | Activity | Std |
| 21311231 | 0.0045 | 0.0368 | 0.0363 | 0.0234 | 0.0015 | 0.1292 | 0.0336 | 0.0063 |
| 21311233 | 0.1702 | 0.0009 | 0.1155 | 0.0108 | 0.2556 | 0.0089 | 0.0619 | 0.0019 |
| 21312133 | 0.1219 | 0.0074 | 0.1157 | 0.0081 | 0.0988 | 0.0037 | 0.0632 | 0.0028 |
| 21312231 | 0.0577 | 0.0040 | 0.0694 | 0.0029 | 0.1105 | 0.0557 | 0.0492 | 0.0031 |
| 21312311 | 0.0951 | 0.0156 | 0.0988 | 0.0027 | 0.2117 | 0.0100 | 0.0475 | 0.0014 |
| 21312332 | 0.0935 | 0.0067 | 0.0973 | 0.0097 | 0.0840 | 0.0088 | 0.0764 | 0.0053 |
| 21313233 | 0.0884 | 0.0030 | 0.0822 | 0.0055 | 0.1462 | 0.0112 | 0.0409 | 0.0063 |
| 21313331 | 0.0789 | 0.0018 | 0.0986 | 0.0057 | 0.1054 | 0.0107 | 0.0347 | 0.0027 |
| 21313333 | 0.0511 | 0.0060 | 0.0582 | 0.0049 | 0.2544 | 0.0885 | 0.0168 | 0.0145 |
| 22311233 | 0.1278 | 0.0065 | 0.0949 | 0.0075 | 0.2365 | 0.0199 | 0.0536 | 0.0034 |
| 22312233 | 0.1018 | 0.0006 | 0.0986 | 0.0078 | 0.1983 | 0.0131 | 0.0672 | 0.0016 |
| 22313331 | 0.2417 | 0.0326 | 0.1130 | 0.0045 | 0.4617 | 0.0085 | 0.0461 | 0.0057 |
| 22313231 | 0.1370 | 0.0109 | 0.0780 | 0.0056 | 0.3916 | 0.0125 | 0.0322 | 0.0021 |
| 22312331 | 0.1180 | 0.0059 | 0.0786 | 0.0049 | 0.2890 | 0.0097 | 0.0386 | 0.0031 |
| 21312331 | 0.0598 | 0.0403 | 0.0848 | 0.0047 | 0.1082 | 0.0070 | 0.0574 | 0.0045 |
| 21312313 | 0.1122 | 0.0036 | 0.1000 | 0.0046 | 0.2181 | 0.0100 | 0.0728 | 0.0013 |
| 22311333 | 0.0328 | 0.0260 | 0.0637 | 0.0064 | 0.0642 | 0.0185 | 0.0560 | 0.0054 |
| 22313333 | 0.1668 | 0.0084 | 0.0914 | 0.0085 | 0.4988 | 0.0143 | 0.0319 | 0.0026 |
| 21112333 | 0.0733 | 0.0033 | 0.0868 | 0.0064 | 0.1453 | 0.0110 | 0.0577 | 0.0030 |
| 21112233 | 0.0680 | 0.0136 | 0.0708 | 0.0046 | 0.1018 | 0.0028 | 0.0518 | 0.0043 |
| 21113333 | 0.1889 | 0.0152 | 0.1937 | 0.0239 | 0.3159 | 0.0165 | 0.1302 | 0.0064 |
| 21112331 | 0.0572 | 0.0036 | 0.0627 | 0.0024 | 0.0467 | 0.0503 | 0.0488 | 0.0023 |
| 22112233 | 0.1679 | 0.0080 | 0.1685 | 0.0089 | 0.3189 | 0.0033 | 0.0884 | 0.0040 |
| 21312213 | 0.2269 | 0.0287 | 0.0907 | 0.0023 | 0.2751 | 0.0154 | 0.0279 | 0.0091 |
| 21311333 | 0.0299 | 0.0266 | 0.0575 | 0.0063 | 0.1293 | 0.0117 | 0.0403 | 0.0056 |
| 21313313 | 0.0757 | 0.0030 | 0.0950 | 0.0084 | 0.3199 | 0.0038 | 0.0480 | 0.0019 |
| 22311331 | 0.1367 | 0.0075 | 0.1018 | 0.0059 | 0.5061 | 0.0242 | 0.0432 | 0.0018 |
| 21312211 | 0.1719 | 0.0239 | 0.1015 | 0.0102 | 0.2824 | 0.0138 | 0.0385 | 0.0051 |
| 21212233 | 0.0945 | 0.0021 | 0.0998 | 0.0069 | 0.1550 | 0.0098 | 0.0646 | 0.0055 |
| 22212333 | 0.1019 | 0.0006 | 0.1052 | 0.0104 | 0.1895 | 0.0078 | 0.0873 | 0.0075 |
| 21311311 | 0.0908 | 0.0019 | 0.1064 | 0.0045 | 0.1765 | 0.0276 | 0.0423 | 0.0012 |
| 21311313 | 0.1934 | 0.0256 | 0.2061 | 0.0211 | 0.3869 | 0.0230 | 0.0876 | 0.0103 |
| 21311331 | −0.7582 | 0.9064 | 0.0549 | 0.0492 | −0.0689 | 0.2017 | 0.0414 | 0.0174 |
| 21313231 | 0.1475 | 0.0072 | 0.1725 | 0.0183 | 0.2191 | 0.0209 | 0.1055 | 0.0095 |
| 22312333 | 0.2075 | 0.0111 | 0.1792 | 0.0181 | 0.2756 | 0.0218 | 0.0907 | 0.0098 |
| 22313233 | 0.0911 | 0.0113 | 0.0872 | 0.0079 | 0.2282 | 0.0058 | 0.0504 | 0.0054 |
| 21212333 | 0.1074 | 0.0051 | 0.1141 | 0.0142 | 0.2192 | 0.0128 | 0.0861 | 0.0086 |
| 21312333 | 0.1027 | 0.0140 | 0.1063 | 0.0097 | 0.1712 | 0.0007 | 0.0724 | 0.0071 |
| 11111111 | 0.0598 | 0.0031 | 0.0985 | 0.0109 | 0.0688 | 0.0082 | 0.0381 | 0.0015 |

TABLE 10
Average monooxygenase activities (in absorbance
units) and standard deviations (based on three parallel
measurements) of holoenzymes on 9 substrates.

| | S1 | | S2 | | S3 | | S4 | | S5 | |
|---|---|---|---|---|---|---|---|---|---|---|
| sequence | Activity | Std | Activity | Std | Activity | Std | Activity | Std | Activity | Std |
| 21311231R1 | 0.2889 | 0.0091 | 0.1448 | 0.0020 | 0.1440 | 0.0061 | 0.1440 | 0.0061 | 0.1416 | 0.0085 |
| 21311233R1 | 0.1103 | 0.0058 | 0.0962 | 0.0006 | 0.1075 | 0.0049 | 0.1075 | 0.0049 | 0.0753 | 0.0028 |
| 21312133R1 | 0.1700 | 0.0143 | 0.1245 | 0.0051 | 0.1518 | 0.0059 | 0.1518 | 0.0059 | 0.1692 | 0.0138 |
| 21312231R1 | 0.0771 | 0.0062 | 0.0948 | 0.0022 | 0.0988 | 0.0003 | 0.0988 | 0.0003 | 0.0600 | 0.0033 |
| 21312311R1 | 0.0418 | 0.0090 | 0.1789 | 0.0088 | 0.1680 | 0.0124 | 0.1680 | 0.0124 | 0.2192 | 0.0261 |
| 21312332R1 | 0.3768 | 0.0303 | 0.1066 | 0.0026 | 0.1260 | 0.0062 | 0.1260 | 0.0062 | 0.0946 | 0.0082 |
| 21313233R1 | 0.1249 | 0.0336 | 0.0944 | 0.0015 | 0.0980 | 0.0006 | 0.0980 | 0.0006 | 0.0748 | 0.0021 |
| 21313331R1 | 0.2754 | 0.0349 | 0.1642 | 0.0033 | 0.1751 | 0.0043 | 0.1751 | 0.0043 | 0.2449 | 0.0295 |
| 21313333R1 | 0.1341 | 0.0058 | 0.1192 | 0.0027 | 0.1444 | 0.0018 | 0.1444 | 0.0018 | 0.2090 | 0.0022 |
| 22311233R1 | 0.2840 | 0.0054 | 0.1581 | 0.0009 | 0.1689 | 0.0021 | 0.1689 | 0.0021 | 0.1490 | 0.0036 |
| 22312233R1 | 0.0599 | 0.0042 | 0.1127 | 0.0016 | 0.1197 | 0.0021 | 0.1197 | 0.0021 | 0.0958 | 0.0023 |
| 22313231R1 | 0.0652 | 0.0069 | 0.1010 | 0.0010 | 0.1036 | 0.0030 | 0.1036 | 0.0030 | 0.0693 | 0.0009 |
| 22312331R1 | 0.0498 | 0.0220 | 0.0857 | 0.0021 | 0.0922 | 0.0001 | 0.0922 | 0.0001 | 0.0597 | 0.0016 |
| 21312331R1 | 0.0764 | 0.0180 | 0.0861 | 0.0009 | 0.1246 | 0.0039 | 0.1246 | 0.0039 | 0.3405 | 0.0110 |
| 21312313R1 | 0.1150 | 0.0095 | 0.1254 | 0.0051 | 0.1436 | 0.0038 | 0.1436 | 0.0038 | 0.1726 | 0.0038 |
| 22311333R1 | 0.0648 | 0.0111 | 0.2069 | 0.0030 | 0.2380 | 0.0030 | 0.2380 | 0.0030 | 0.2198 | 0.0018 |
| 22313333R1 | 0.0482 | 0.0035 | 0.3417 | 0.0015 | 0.3302 | 0.0059 | 0.3302 | 0.0059 | 0.2743 | 0.0050 |
| 21112333R1 | 0.0751 | 0.0042 | 0.1100 | 0.0009 | 0.1257 | 0.0010 | 0.1257 | 0.0010 | 0.1801 | 0.0034 |
| 21112233R1 | 0.0898 | 0.0024 | 0.0849 | 0.0014 | 0.0935 | 0.0007 | 0.0935 | 0.0007 | 0.0773 | 0.0078 |
| 21113333R1 | 0.1297 | 0.0096 | 0.1151 | 0.0025 | 0.1438 | 0.0140 | 0.1438 | 0.0140 | 0.1192 | 0.0073 |
| 21112331R1 | 0.0617 | 0.0060 | 0.1670 | 0.0042 | 0.1478 | 0.0034 | 0.1478 | 0.0034 | 0.2785 | 0.0031 |
| 22112333R1 | 0.0893 | 0.0088 | 0.2075 | 0.0018 | 0.2721 | 0.0043 | 0.2721 | 0.0043 | 0.2795 | 0.0040 |
| 22112233R1 | 0.1387 | 0.0531 | 0.1426 | 0.0011 | 0.1840 | 0.0122 | 0.1840 | 0.0122 | 0.1268 | 0.0002 |
| 21312213R1 | 0.0664 | 0.0094 | 0.1786 | 0.0051 | 0.2163 | 0.0059 | 0.2163 | 0.0059 | 0.1957 | 0.0048 |
| 21311333R1 | 0.1035 | 0.0138 | 0.2833 | 0.0039 | 0.3527 | 0.0069 | 0.3527 | 0.0069 | 0.3871 | 0.0018 |
| 21313313R1 | 0.1333 | 0.0386 | 0.1329 | 0.0019 | 0.1530 | 0.0034 | 0.1530 | 0.0034 | 0.1282 | 0.0089 |
| 21312211R1 | 0.1429 | 0.0468 | 0.0678 | 0.0009 | 0.0870 | 0.0021 | 0.0870 | 0.0021 | 0.0616 | 0.0012 |
| 21212233R1 | 0.1548 | 0.0053 | 0.1352 | 0.0020 | 0.2002 | 0.0027 | 0.2002 | 0.0027 | 0.3289 | 0.0041 |
| 22212333R1 | 0.1032 | 0.0213 | 0.1112 | 0.0027 | 0.1230 | 0.0013 | 0.1230 | 0.0013 | 0.1233 | 0.0014 |
| 21311311R1 | 0.0785 | 0.0143 | 0.1754 | 0.0058 | 0.2046 | 0.0091 | 0.2046 | 0.0091 | 0.1851 | 0.0050 |
| 21311313R1 | 0.1719 | 0.0383 | 0.1628 | 0.0021 | 0.2250 | 0.0013 | 0.2250 | 0.0013 | 0.3040 | 0.0022 |
| 21311331R1 | 0.1630 | 0.0384 | 0.1247 | 0.0051 | 0.1509 | 0.0026 | 0.1509 | 0.0026 | 0.1833 | 0.0006 |
| 21313231R1 | 0.0784 | 0.0323 | 0.1594 | 0.0063 | 0.1962 | 0.0124 | 0.1962 | 0.0124 | 0.1554 | 0.0077 |
| 22312231R1 | 0.0140 | 0.0137 | 0.1361 | 0.0019 | 0.1889 | 0.0075 | 0.1889 | 0.0075 | 0.2877 | 0.0087 |
| 22312333R1 | 0.0770 | 0.0165 | 0.1703 | 0.0080 | 0.2483 | 0.0114 | 0.2483 | 0.0114 | 0.2941 | 0.0183 |
| 22313233R1 | 0.1238 | 0.0140 | 0.1434 | 0.0043 | 0.1955 | 0.0040 | 0.1955 | 0.0040 | 0.1395 | 0.0061 |
| 21212333R1 | 0.0281 | 0.0023 | 0.1328 | 0.0090 | 0.1838 | 0.0008 | 0.1838 | 0.0008 | 0.2975 | 0.0026 |
| 21312333R1 | 0.1237 | 0.0086 | 0.0277 | 0.0012 | 0.1675 | 0.0025 | 0.1675 | 0.0025 | 0.2544 | 0.0047 |
| 11111111R1 | 0.4650 | 0.2322 | 0.3212 | 0.0040 | 0.2286 | 0.0132 | 0.2286 | 0.0132 | 0.3322 | 0.0107 |

| | S6 | | S7 | | S8 | | S9 | |
|---|---|---|---|---|---|---|---|---|
| sequence | Activity | Std | Activity | Std | Activity | Std | Activity | Std |
| 21311231R1 | 0.3967 | 0.0049 | 0.0616 | 0.0006 | 0.0616 | 0.0033 | 0.0541 | 0.0011 |
| 21311233R1 | 0.1074 | 0.0056 | 0.0673 | 0.0006 | 0.0686 | 0.0011 | 0.0538 | 0.0015 |
| 21312133R1 | 0.1912 | 0.0125 | 0.0761 | 0.0011 | 0.0816 | 0.0045 | 0.0648 | 0.0084 |
| 21312231R1 | 0.0747 | 0.0050 | 0.0646 | 0.0009 | 0.0584 | 0.0018 | 0.0458 | 0.0027 |
| 21312311R1 | 0.2283 | 0.0141 | 0.0623 | 0.0020 | 0.0721 | 0.0013 | 0.0504 | 0.0020 |
| 21312332R1 | 0.0912 | 0.0060 | 0.0985 | 0.0043 | 0.0921 | 0.0025 | 0.0787 | 0.0020 |
| 21313233R1 | 0.0839 | 0.0043 | 0.0642 | 0.0017 | 0.0936 | 0.0109 | 0.0505 | 0.0007 |
| 21313331R1 | 0.3340 | 0.0115 | 0.0731 | 0.0055 | 0.1152 | 0.0035 | 0.0642 | 0.0042 |
| 21313333R1 | 0.2454 | 0.0087 | 0.0557 | 0.0054 | 0.0977 | 0.0093 | 0.0495 | 0.0016 |
| 22311233R1 | 0.3693 | 0.0027 | 0.0617 | 0.0020 | 0.0841 | 0.0067 | 0.0509 | 0.0016 |
| 22312233R1 | 0.1098 | 0.0022 | 0.0734 | 0.0024 | 0.0973 | 0.0032 | 0.0665 | 0.0021 |
| 22313231R1 | 0.0780 | 0.0034 | 0.0764 | 0.0058 | 0.0696 | 0.0034 | 0.0604 | 0.0010 |
| 22312331R1 | 0.0604 | 0.0035 | 0.0653 | 0.0034 | 0.0597 | 0.0014 | 0.0511 | 0.0029 |
| 21312331R1 | 0.1971 | 0.0017 | 0.0644 | 0.0032 | 0.0605 | 0.0018 | 0.0534 | 0.0013 |
| 21312313R1 | 0.1443 | 0.0024 | 0.1088 | 0.0022 | 0.1011 | 0.0017 | 0.0876 | 0.0023 |
| 22311333R1 | 0.3530 | 0.0060 | 0.0758 | 0.0009 | 0.0990 | 0.0035 | 0.0699 | 0.0010 |
| 22313333R1 | 0.4823 | 0.0512 | 0.0662 | 0.0019 | 0.1367 | 0.0094 | 0.0605 | 0.0050 |
| 21112333R1 | 0.1692 | 0.0111 | 0.0625 | 0.0029 | 0.0718 | 0.0086 | 0.0527 | 0.0021 |
| 21112233R1 | 0.0682 | 0.0017 | 0.0629 | 0.0020 | 0.0661 | 0.0045 | 0.0530 | 0.0017 |
| 21113333R1 | 0.1157 | 0.0092 | 0.0980 | 0.0004 | 0.0967 | 0.0008 | 0.0858 | 0.0013 |
| 21112331R1 | 0.2512 | 0.0081 | 0.0941 | 0.0063 | 0.1161 | 0.0050 | 0.0697 | 0.0031 |
| 22112333R1 | 0.3460 | 0.1748 | 0.1385 | 0.0037 | 0.1772 | 0.0057 | 0.1210 | 0.0054 |
| 22112233R1 | 0.1286 | 0.0056 | 0.1245 | 0.0031 | 0.1424 | 0.0050 | 0.1119 | 0.0010 |
| 21312213R1 | 0.1662 | 0.0150 | 0.1763 | 0.0041 | 0.1587 | 0.0137 | 0.1575 | 0.0032 |
| 21311333R1 | 0.4763 | 0.0124 | 0.1575 | 0.0017 | 0.2645 | 0.0015 | 0.1345 | 0.0019 |
| 21313313R1 | 0.1156 | 0.0045 | 0.1185 | 0.0094 | 0.1121 | 0.0061 | 0.0982 | 0.0023 |
| 21312211R1 | 0.0553 | 0.0074 | 0.0506 | 0.0016 | 0.0548 | 0.0016 | 0.0464 | 0.0012 |
| 21212233R1 | 0.3414 | 0.0029 | 0.0862 | 0.0030 | 0.0953 | 0.0036 | 0.0669 | 0.0014 |
| 22212333R1 | 0.1098 | 0.0011 | 0.0955 | 0.0041 | 0.0878 | 0.0048 | 0.0796 | 0.0026 |
| 21311311R1 | 0.1696 | 0.0145 | 0.1832 | 0.0014 | 0.1600 | 0.0019 | 0.1456 | 0.0014 |
| 21311313R1 | 0.2209 | 0.0069 | 0.1255 | 0.0042 | 0.1477 | 0.0056 | 0.1072 | 0.0035 |
| 21311331R1 | 0.1111 | 0.0030 | 0.0995 | 0.0034 | 0.1045 | 0.0047 | 0.0910 | 0.0052 |
| 21313231R1 | 0.1712 | 0.0034 | 0.1528 | 0.0022 | 0.1544 | 0.0012 | 0.1224 | 0.0027 |
| 22312231R1 | 0.3059 | 0.0082 | 0.0709 | 0.0019 | 0.0728 | 0.0034 | 0.0547 | 0.0029 |
| 22312333R1 | 0.3658 | 0.0045 | 0.1217 | 0.0032 | 0.1233 | 0.0142 | 0.0926 | 0.0014 |
| 22313233R1 | 0.2749 | 0.0212 | 0.0940 | 0.0013 | 0.2227 | 0.0084 | 0.0738 | 0.0018 |
| 21212333R1 | 0.2039 | 0.0024 | 0.1001 | 0.0118 | 0.1260 | 0.0047 | 0.0882 | 0.0044 |
| 21312333R1 | 0.1868 | 0.0048 | 0.1021 | 0.0023 | 0.1231 | 0.0049 | 0.0876 | 0.0010 |
| 11111111R1 | 0.5281 | 0.0063 | 0.0759 | 0.0010 | 0.0865 | 0.0036 | 0.0535 | 0.0004 |

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s)

### REFERENCES

1. DePristo, M. A., Weinreich, D. M. & Hartl, D. L. Missense meanderings in sequence space: A biophysical view of protein evolution. Nat. Rev. Genet. 6, 678-687 (2005).
2. Yue, P., Li, Z. L. & Moult, J. Loss of protein structure stability as a major causative factor in monogenic disease. J. Mol. Biol. 353, 459-473 (2005).
3. Bloom, J. D. et al. Thermodynamic prediction of protein neutrality. Proc. Nat. Acad. Sci. USA 102, 606-611 (2005).
4. Bloom, J. D., Labthavikul, S. T., Otey, C. R. & Arnold, F. H. Protein stability promotes evolvability Proc. Nat. Acad. Sci. USA 103, 5869-5874 (2006).
5. Drummond, D. A., Bloom, J. D., Adami, C., Wilke, C. O. & Arnold, F. H. Why highly expressed proteins evolve slowly. Proc. Nat. Acad. Sci. USA 102, 14338-14343 (2005).
6. Niehaus, F., Bertoldo, C., Kahler, M. & Antranikian, G. Extremophiles as a source of novel enzymes for industrial application. Appl. Microbiol. Biot. 51, 711-729 (1999).
7. Zeikus, J. G., Vieille, C. & Savchenko, A. Thermozymes: biotechnology and structure-function relationships. Extremophiles 2, 179-183 (1998).
8. Guengerich, F. P. Cytochrome P450 enzymes in the generation of commercial products. Nat. Rev. Drug Discov. 1, 359-366 (2002).
9. Landwehr, M. et al. Enantioselective alpha-hydroxylation of 2-arylacetic acid derivatives and buspirone catalyzed by engineered cytochrome P450BM-3. J. Am. Chem. Soc. 128, 6058-6059 (2006).
10. Otey, C. R., Bandara, G., Lalonde, J., Takahashi, K. & Arnold, F. H. Preparation of human metabolites of propranolol using laboratory-evolved bacterial cytochromes P450. Biotechnol. Bioeng. 93, 494-499 (2006).
11. Urlacher, V. B. & Eiben, S. Cytochrome P450 monooxygenases: perspectives for synthetic application. Trends Biotechnol. 24, 324-330 (2006).
12. van Vugt-Lussenburg, B. M. A. et al. Heterotropic and homotropic cooperativity by a drug-metabolising mutant of cytochrome P450BM3. Biochem. Bioph. Res. Comm. 346, 810-818 (2006).
13. Otey, C. R. et al. Structure-guided recombination creates an artificial family of cytochromes P450. PLoS Biol. 4, e112 (2006).
14. Dietterich, T. G. Approximate statistical tests for comparing supervised classification learning algorithms. Neural Comput. 10, 1895-1923 (1998).
15. Fox, R. et al. Optimizing the search algorithm for protein engineering by directed evolution. Protein Eng. 16, 589-597 (2003).
16. Amin, N. et al. Construction of stabilized proteins by combinatorial consensus mutagenesis. Protein Eng. Des. Sel. 17, 787-793 (2004).
17. Lehmann, M. et al. The consensus concept for thermostability engineering of proteins: further proof of concept. Protein Eng. 15, 403-411 (2002).
18. Steipe, B., Schiller, B., Pluckthun, A. & Steinbacher, S. Sequence statistics reliably predict stabilizing mutations in a protein domain. J. Mol. Biol. 240, 188-192 (1994).
19. Joern, J. M., Meinhold, P. & Arnold, F. H. Analysis of shuffled gene libraries. J. Mol. Biol. 316, 643-656 (2002).
20. Johannes, T. W., Woodyer, R. D., & Zhao, H. M. Directed evolution of a thermostable phosphite dehydrogenase for NAD(P)H regeneration. Appl. Environ. Microb. 71, 5728-5734 (2005)
21. Landwehr, M., Carbone, M., Otey, C. R., Li, Y. & Arnold, F. H. Diversification of catalytic function in a synthetic family of chimeric cytochrome P450s. Chem. Biol. In press (2007).
22. Somero, G. N. Proteins and temperature. Annu. Rev. Physiol. 57, 43-68 (1995).
23. Arnold, F. H., Wintrode, P. L., Miyazaki, K. & Gershenson, A. How enzymes adapt: lessons from directed evolution. Trends Biochem. Sci. 26, 100-106 (2001).
24. Taverna, D. M. & Goldstein, R. A. Why are proteins marginally stable? Proteins 46, 105-109 (2002).
25. Bloom, J. D., Raval, A. & Wilke, C. O. Thermodynamics of neutral protein evolution Genetics 175, 255-266 (2007).
26. Serrano, L., Day, A. G. & Fersht, A. R. Step-wise mutation of barnase to binase—a procedure for engineering increased stability of proteins and an experimental-analysis of the evolution of protein stability. J. Mol. Biol. 233, 305-312 (1993).
27. Giver, L., Gershenson, A., Freskgard, P. O. & Arnold, F. H. Directed evolution of a thermostable esterase. Proc. Nat. Acad. Sci. USA 95, 12809-12813 (1998).

1. A polypeptide comprising:
a heme domain and a reductase domain;
the heme domain comprising from N- to C-terminus: (segment 1)-(segment 2)-(segment 3)-(segment 4)-(segment 5)-(segment 6)-(segment 7)-(segment 8);
wherein:
segment 1 is amino acid residue from about 1 to about x₁ of SEQ ID NO:1 (“1”), SEQ ID NO:2 (“2”) or SEQ ID NO:3 (“3”);
segment 2 is from about amino acid residue x₁ to about x₂ of SEQ ID NO:1 (“1”), SEQ ID NO:2 (“2”) or SEQ ID NO:3 (“3”);
segment 3 is from about amino acid residue x₂ to about x₃ of SEQ ID NO:1 (“1”), SEQ ID NO:2 (“2”) or SEQ ID NO:3 (“3”);
segment 4 is from about amino acid residue x₃ to about x₄ of SEQ ID NO:1 (“1”), SEQ ID NO:2 (“2”) or SEQ ID NO:3 (“3”);
segment 5 is from about amino acid residue x₄ to about x₅ of SEQ ID NO:1 (“1”), SEQ ID NO:2 (“2”) or SEQ ID NO:3 (“3”);
segment 6 is from about amino acid residue x₅ to about x₆ of SEQ ID NO:1 (“1”), SEQ ID NO:2 (“2”) or SEQ ID NO:3 (“3”);
segment 7 is from about amino acid residue x₆ to about x₇ of SEQ ID NO:1 (“1”), SEQ ID NO:2 (“2”) or SEQ ID NO:3 (“3”); and
segment 8 is from about amino acid residue x₇ to about x₈ of SEQ ID NO:1 (“1”), SEQ ID NO:2 (“2”) or SEQ ID NO:3 (“3”);
wherein:
x₁ is residue 62, 63, 64, 65 or 66 of SEQ ID NO:1, or residue 63, 64, 65, 66 or 67 of SEQ ID NO:2 or SEQ ID NO:3;
x₂ is residue 120, 121, 122, 123, 124, 125, 126, 127, 128, 129, 130, 132 or 132 of SEQ ID NO:1, or residue 121, 122, 123, 124, 125, 126, 127, 128, 129, 130, 131, 132, or 133 of SEQ ID NO:2 or SEQ ID NO:3;
x₃ is residue 164, 165, 166, 167, 168, 169, 170, 171, 172, 173, 174, 175, 176, or 177 of SEQ ID NO:1, or residue 165, 166, 167, 168, 169, 170, 171, 172, 173, 174, 175, 176, 177, or 178 of SEQ ID NO:2 or SEQ ID NO:3;
x₄ is residue 214, 215, 216, 217 or 218 of SEQ ID NO:1, or residue 215, 216, 217, 218 or 219 of SEQ ID NO:2 or SEQ ID NO:3;
x₅ is residue 266, 267, 268, 269 or 270 of SEQ ID NO:1, or residue 268, 269, 270, 271 or 272 of SEQ ID NO:2 or SEQ ID NO:3;
x₆ is residue 326, 327, 328, 329 or 330 of SEQ ID NO:1, or residue 328, 329, 330, 331 or 332 of SEQ ID NO:2 or SEQ ID NO:3;
x₇ is residue 402, 403, 404, 405 or 406 of SEQ ID NO:1, or residue 404, 405, 405, 407 or 408 of SEQ ID NO:2 or SEQ ID NO:3; and
x₈ is an amino acid residue corresponding to the C-terminus of the heme domain of CYP102A1, CYP102A2 or CYP102A3 or the C-terminus of SEQ ID NO:1, SEQ ID NO:2 or SEQ ID NO:3;
wherein the heme domain has a general structure selected from the group consisting of:
11112212, 11113233, 11113311, 11131313, 11132223, 11132232, 11133231, 11212112, 11212333, 11213133, 11213231, 11232111, 11232232, 11232333, 11311233, 11312233, 11313233, 11313333, 11331312, 11331333, 11332212, 11332233, 11332333, 11333212, 12112333, 12113221, 12211232, 12211333, 12212112, 12212211, 12212212, 12212223, 12212332, 12213212, 12232111, 12232112, 12232232, 12232233, 12232332, 12233112, 12233212, 12313331, 12322333, 12331123, 12331333, 12332223, 12332333, 12333331, 12333333, 13113311, 13213131, 13221231, 13222212, 13233212, 13332333, 13333122, 13333132, 13333211, 13333233, 21111321, 21111323, 21111333, 21112122, 21112123, 21112132, 21112212, 21112222, 21112232, 21112233, 21112311, 21112312, 21112331, 21112332, 21112333, 21113111, 21113112, 21113122, 21113133, 21113211, 21113212, 21113221, 21113223, 21113312, 21113321, 21113322, 21113333, 21131121, 21132112, 21132113, 21132212, 21132222, 21132311, 21132313, 21132321, 21132323, 21133112, 21133113, 21133131, 21133211, 21133222, 21133223, 21133232, 21133233, 21133312, 21133313, 21133321, 21133322, 21133331, 21133332, 21211223, 21211321, 21212111, 21212112, 21212122, 21212123, 21212133, 21212212, 21212213, 21212231, 21212233, 21212321, 21212332, 21212333, 21213121, 21213212, 21213223, 21213231, 21213321, 21213332, 21222112, 21231232, 21231233, 21232112, 21232122, 21232132, 21232212, 21232222, 21232231, 21232232, 21232233, 21232321, 21232322, 21232323, 21232332, 21233111, 21233132, 21233212, 21233221, 21233233, 21233312, 21233321, 21311122, 21311223, 21311231, 21311233, 21311311, 21311313, 21311331, 21311333, 21312111, 21312112, 21312122, 21312123, 21312133, 21312211, 21312213, 21312222, 21312223, 21312231, 21312233, 21312311, 21312313, 21312321, 21312322, 21312323, 21312331, 21312332, 21312333, 21313111, 21313112, 21313122, 21313221, 21313231, 21313233, 21313311, 21313312, 21313313, 21313322, 21313331, 21313333, 21331223, 21331332, 21331333, 21332111, 21332112, 21332113, 21332122, 21332131, 21332212, 21332221, 21332223, 21332231, 21332233, 21332312, 21332322, 21332323, 21332331, 21332332, 21332333, 21333111, 21333122, 21333131, 21333132, 21333211, 21333212, 21333221, 21333223, 21333233, 21333312, 21333321, 22313333, 21333333, 22111223, 22111332, 22112111, 22112131, 22112211, 22112223, 22112233, 22112321, 22112323, 22112331, 22112333, 22113111, 22113211, 22113223, 22113232, 22113233, 22113313, 22113323, 22113332, 22131221, 22132112, 22132113, 22132212, 22132231, 22132233, 22132312, 22132323, 22132331, 22133112, 22133211, 22133212, 22133232, 22133312, 22133322, 22133323, 22212111, 22212123, 22212131, 22212212, 22212232, 22212312, 22212321, 22212322, 22212333, 22213111, 22213112, 22213132, 22213212, 22213222, 22213223, 22213312, 22213321, 22222121, 22231221, 22231223, 22231312, 22231322, 22232111, 22232112, 22232121, 22232122, 22232123, 22232212, 22232222, 22232223, 22232232, 22232233, 22232311, 22232312, 22232322, 22232323, 22232331, 22232333, 22233112, 22233211, 22233212, 22233221, 22233222, 22233223, 22233312, 22233323, 22233332, 22311123, 22311212, 22311231, 22311233, 22311331, 22311333, 22312111, 22312123, 22312132, 22312133, 22312211, 22312221, 22312222, 22312223, 22312231, 22312232, 22312233, 22312311, 22312312, 22312322, 22312331, 22312332, 22312333, 22313122, 22313212, 22313221, 22313222, 22313231, 22313232, 22313233, 22313323, 22313331, 22313332, 22323313, 22331123, 22331133, 22331221, 22331223, 22331323, 22331332, 22332112, 22332113, 22332121, 22332123, 22332132, 22332211, 22332221, 22332222, 22332223, 22332232, 22332233, 22332312, 22332321, 22332322, 22332332, 22333112, 22333122, 22333131, 22333132, 22333133, 22333211, 22333212, 22333221, 22333222, 22333223, 22333231, 22333311, 22333313, 22333321, 22333323, 22333332, 23112213, 23112221, 23112223, 23112233, 23112323, 23112333, 23113111, 23113112, 23113121, 23113131, 23113212, 23113311, 23113312, 23113323, 23113332, 23122212, 23131323, 23132111, 23132121, 23132212, 23132221, 23132232, 23132233, 23132311, 23132322, 23132323, 23133112, 23133113, 23133121, 23133233, 23133311, 23133321, 23133331, 23133333, 23211132, 23212112, 23212211, 23212212, 23212221, 23212222, 23212231, 23212332, 23212333, 23213112, 23213121, 23213123, 23213211, 23213212, 23213223, 23213232, 23213311, 23213322, 23213333, 23231233, 23232113, 23232131, 23232211, 23232212, 23232311, 23232323, 23233212, 23233221, 23233231, 23233232, 23233312, 23233333, 23311233, 23311323, 23312112, 23312121, 23312122, 23312123, 23312131, 23312223, 23312311, 23312312, 23312323, 23313111, 23313133, 23313212, 23313222, 23313232, 23313233, 23313323, 23313333, 23331233, 23331323, 23332112, 23332221, 23332222, 23332223, 23332231, 23332311, 23332323, 23332331, 23333111, 23333123, 23333131, 23333211, 23333212, 23333213, 23333222, 23333223, 23333232, 23333233, 23333311, 23333312, 23333323, 31111233, 31112231, 31112333, 31113131, 31113132, 31113222, 31113323, 31113331, 31113332, 31131233, 31132231, 31132232, 31132333, 31133233, 31133331, 31211131, 31211232, 31212112, 31212212, 31212232, 31212321, 31212323, 31212331, 31212332, 31212333, 31213232, 31213233, 31213323, 31213331, 31213332, 31232231, 31232312, 31232333, 31233221, 31233222, 31233233, 31311231, 31311233, 31311332, 31312113, 31312133, 31312212, 31312222, 31312231, 31312233, 31312323, 31312332, 31312333, 31313111, 31313131, 31313132, 31313133, 31313223, 31313232, 31313233, 31313333, 31331331, 31331333, 31332131, 31332133, 31332232, 31332233, 31332312, 31332322, 31332323, 31332333, 31333233, 31333322, 31333332, 31333333, 32111333, 32112212, 32112313, 32112321, 32113131, 32113232, 32113233, 32131133, 32132232, 32132233, 32132331, 32133111, 32133232, 32133233, 32133331, 32211323, 32212133, 32212231, 32212232, 32212233, 32212321, 32212323, 32212332, 32212333, 32213123, 32213132, 32213231, 32213333, 32232131, 32232322, 32232331, 32232333, 32233222, 32233332, 32311131, 32311323, 32312212, 32312231, 32312233, 32312311, 32312322, 32312323, 32312331, 32312332, 32312333, 32313133, 32313231, 32313232, 32313233, 32313313, 32313332, 32313333, 32332133, 32332223, 32332231, 32332232, 32332322, 32332323, 32332331, 32332332, 32332333, 32333223, 32333232, 32333233, 32333312, 32333323, 32333333, 33113111, 33113211, 33113212, 33113233, 33131333, 33133131, 33133333, 33212213, 33212311, 33212333, 33213211, 33213232, 33213333, 33232233, 33232312, 33232333, 33233131, 33233233, 33233333, 33311231, 33312133, 33312322, 33312333, 33313223, 33313233, 33313323, 33313333, 33331232, 33331233, 33331333, 33332131, 33332133, 33332221, 33332232, 33332233, 33332323, 33332333, 33333123, 33333231, 33333232, 33333233, 33333321, and 33333323,
wherein the reductase domain comprises at least 50% identity to the reductase domain of SEQ ID NO:1, 2 or 3 and wherein the polypeptide has monooxygenase activity.

2. The polypeptide of claim 1, wherein the heme domain is selected from the group consisting of:
21112233, 21112331, 21112333, 21113333, 21212233, 21212333, 21311231, 21311233, 21311311, 21311313, 21311331, 21311333, 21312133, 21312211, 21312213, 21312231, 21312311, 21312313, 21312331, 21312332, 21312333, 21313231, 21313233, 21313313, 21313331, 21313333, 22112233, 22112333, 22212333, 22311233, 22311331, 22311333, 22312231, 22312233, 22312331, 22312333, 22313231, 22313233, 22313331, and 22313333.

3. The polypeptide of claim 1, wherein the heme domain has a CO-binding peak at 450 nm.

4. The polypeptide of claim 1, wherein the polypeptide has improved monooxygenase activity compared to a wild-type polypeptide consisting of SEQ ID NO:1, 2, or 3.

5. The polypeptide of claim 1, wherein the reductase domain comprises the reductase domain of SEQ ID NO:1, and wherein the polypeptide has monooxygenase activity.

6. The polypeptide of claim 1, wherein the reductase domain comprises the reductase domain of SEQ ID NO:2, and wherein the polypeptide has monooxygenase activity.

7. The polypeptide of claim 1, wherein the substrate specificity of the polypeptide is different compared to the wild-type polypeptide consisting of SEQ ID NO:1, 2, or 3.

8. A polypeptide comprising the general structure from N-terminus to C-terminus
a heme domain comprising (segment 1)-(segment 2)-(segment 3)-(segment 4)-(segment 5)-(segment 6)-(segment 7)-(segment 8); and
a reductase domain,
wherein segment 1 comprises an amino acid sequence from about residue 1 to about x₁ of SEQ ID NO:1 (“1”), SEQ ID NO:2 (“2”) or SEQ ID NO:3 (“3”) and having about 1-10 conservative amino acid substitutions;
segment 2 is from about amino acid residue x₁ to about x₂ of SEQ ID NO:1 (“1”), SEQ ID NO:2 (“2”) or SEQ ID NO:3 (“3”) and having about 1-10 conservative amino acid substitutions;
segment 3 is from about amino acid residue x₂ to about x₃ of SEQ ID NO:1 (“1”), SEQ ID NO:2 (“2”) or SEQ ID NO:3 (“3”) and having about 1-10 conservative amino acid substitutions;
segment 4 is from about amino acid residue x₃ to about x₄ of SEQ ID NO:1 (“1”), SEQ ID NO:2 (“2”) or SEQ ID NO:3 (“3”) and having about 1-10 conservative amino acid substitutions;
segment 5 is from about amino acid residue x₄ to about x₅ of SEQ ID NO:1 (“1”), SEQ ID NO:2 (“2”) or SEQ ID NO:3 (“3”) and having about 1-10 conservative amino acid substitutions;
segment 6 is from about amino acid residue x₅ to about x₆ of SEQ ID NO:1 (“1”), SEQ ID NO:2 (“2”) or SEQ ID NO:3 (“3”) and having about 1-10 conservative amino acid substitutions;
segment 7 is from about amino acid residue x₆ to about x₇ of SEQ ID NO:1 (“1”), SEQ ID NO:2 (“2”) or SEQ ID NO:3 (“3”) and having about 1-10 conservative amino acid substitutions; and
segment 8 is from about amino acid residue x₇ to about x₈ of SEQ ID NO:1 (“1”), SEQ ID NO:2 (“2”) or SEQ ID NO:3 (“3”) and having about 1-10 conservative amino acid substitutions;
wherein:
x₁ is residue 62, 63, 64, 65 or 66 of SEQ ID NO:1, or residue 63, 64, 65, 66 or 67 of SEQ ID NO:2 or SEQ ID NO:3;
x₂ is residue 120, 121, 122, 123, 124, 125, 126, 127, 128, 129, 130, 132 or 132 of SEQ ID NO:1, or residue 121, 122, 123, 124, 125, 126, 127, 128, 129, 130, 131, 132, or 133 of SEQ ID NO:2 or SEQ ID NO:3;
x₃ is residue 164, 165, 166, 167, 168, 169, 170, 171, 172, 173, 174, 175, 176, or 177 of SEQ ID NO:1, or residue 165, 166, 167, 168, 169, 170, 171, 172, 173, 174, 175, 176, 177, or 178 of SEQ ID NO:2 or SEQ ID NO:3;
x₄ is residue 214, 215, 216, 217 or 218 of SEQ ID NO:1, or residue 215, 216, 217, 218 or 219 of SEQ ID NO:2 or SEQ ID NO:3;
x₅ is residue 266, 267, 268, 269 or 270 of SEQ ID NO:1, or residue 268, 269, 270, 271 or 272 of SEQ ID NO:2 or SEQ ID NO:3;
x₆ is residue 326, 327, 328, 329 or 330 of SEQ ID NO:1, or residue 328, 329, 330, 331 or 332 of SEQ ID NO:2 or SEQ ID NO:3;
x₇ is residue 402, 403, 404, 405 or 406 of SEQ ID NO:1, or residue 404, 405, 405, 407 or 408 of SEQ ID NO:2 or SEQ ID NO:3; and
x₈ is an amino acid residue corresponding to the C-terminus of the heme domain of CYP102A1, CYP102A2 or CYP102A3 or the C-terminus of SEQ ID NO:1, SEQ ID NO:2 or SEQ ID NO:3;
wherein the heme domain has a general structure selected from the group consisting of:
11112212, 11113233, 11113311, 11131313, 11132223, 11132232, 11133231, 11212112, 11212333, 11213133, 11213231, 11232111, 11232232, 11232333, 11311233, 11312233, 11313233, 11313333, 11331312, 11331333, 11332212, 11332233, 11332333, 11333212, 12112333, 12113221, 12211232, 12211333, 12212112, 12212211, 12212212, 12212223, 12212332, 12213212, 12232111, 12232112, 12232232, 12232233, 12232332, 12233112, 12233212, 12313331, 12322333, 12331123, 12331333, 12332223, 12332333, 12333331, 12333333, 13113311, 13213131, 13221231, 13222212, 13233212, 13332333, 13333122, 13333132, 13333211, 13333233, 21111321, 21111323, 21111333, 21112122, 21112123, 21112132, 21112212, 21112222, 21112232, 21112233, 21112311, 21112312, 21112331, 21112332, 21112333, 21113111, 21113112, 21113122, 21113133, 21113211, 21113212, 21113221, 21113223, 21113312, 21113321, 21113322, 21113333, 21131121, 21132112, 21132113, 21132212, 21132222, 21132311, 21132313, 21132321, 21132323, 21133112, 21133113, 21133131, 21133211, 21133222, 21133223, 21133232, 21133233, 21133312, 21133313, 21133321, 21133322, 21133331, 21133332, 21211223, 21211321, 21212111, 21212112, 21212122, 21212123, 21212133, 21212212, 21212213, 21212231, 21212233, 21212321, 21212332, 21212333, 21213121, 21213212, 21213223, 21213231, 21213321, 21213332, 21222112, 21231232, 21231233, 21232112, 21232122, 21232132, 21232212, 21232222, 21232231, 21232232, 21232233, 21232321, 21232322, 21232323, 21232332, 21233111, 21233132, 21233212, 21233221, 21233233, 21233312, 21233321, 21311122, 21311223, 21311231, 21311233, 21311311, 21311313, 21311331, 21311333, 21312111, 21312112, 21312122, 21312123, 21312133, 21312211, 21312213, 21312222, 21312223, 21312231, 21312233, 21312311, 21312313, 21312321, 21312322, 21312323, 21312331, 21312332, 21312333, 21313111, 21313112, 21313122, 21313221, 21313231, 21313233, 21313311, 21313312, 21313313, 21313322, 21313331, 21313333, 21331223, 21331332, 21331333, 21332111, 21332112, 21332113, 21332122, 21332131, 21332212, 21332221, 21332223, 21332231, 21332233, 21332312, 21332322, 21332323, 21332331, 21332332, 21332333, 21333111, 21333122, 21333131, 21333132, 21333211, 21333212, 21333221, 21333223, 21333233, 21333312, 21333321, 22313333, 21333333, 22111223, 22111332, 22112111, 22112131, 22112211, 22112223, 22112233, 22112321, 22112323, 22112331, 22112333, 22113111, 22113211, 22113223, 22113232, 22113233, 22113313, 22113323, 22113332, 22131221, 22132112, 22132113, 22132212, 22132231, 22132233, 22132312, 22132323, 22132331, 22133112, 22133211, 22133212, 22133232, 22133312, 22133322, 22133323, 22212111, 22212123, 22212131, 22212212, 22212232, 22212312, 22212321, 22212322, 22212333, 22213111, 22213112, 22213132, 22213212, 22213222, 22213223, 22213312, 22213321, 22222121, 22231221, 22231223, 22231312, 22231322, 22232111, 22232112, 22232121, 22232122, 22232123, 22232212, 22232222, 22232223, 22232232, 22232233, 22232311, 22232312, 22232322, 22232323, 22232331, 22232333, 22233112, 22233211, 22233212, 22233221, 22233222, 22233223, 22233312, 22233323, 22233332, 22311123, 22311212, 22311231, 22311233, 22311331, 22311333, 22312111, 22312123, 22312132, 22312133, 22312211, 22312221, 22312222, 22312223, 22312231, 22312232, 22312233, 22312311, 22312312, 22312322, 22312331, 22312332, 22312333, 22313122, 22313212, 22313221, 22313222, 22313231, 22313232, 22313233, 22313323, 22313331, 22313332, 22323313, 22331123, 22331133, 22331221, 22331223, 22331323, 22331332, 22332112, 22332113, 22332121, 22332123, 22332132, 22332211, 22332221, 22332222, 22332223, 22332232, 22332233, 22332312, 22332321, 22332322, 22332332, 22333112, 22333122, 22333131, 22333132, 22333133, 22333211, 22333212, 22333221, 22333222, 22333223, 22333231, 22333311, 22333313, 22333321, 22333323, 22333332, 23112213, 23112221, 23112223, 23112233, 23112323, 23112333, 23113111, 23113112, 23113121, 23113131, 23113212, 23113311, 23113312, 23113323, 23113332, 23122212, 23131323, 23132111, 23132121, 23132212, 23132221, 23132232, 23132233, 23132311, 23132322, 23132323, 23133112, 23133113, 23133121, 23133233, 23133311, 23133321, 23133331, 23133333, 23211132, 23212112, 23212211, 23212212, 23212221, 23212222, 23212231, 23212332, 23212333, 23213112, 23213121, 23213123, 23213211, 23213212, 23213223, 23213232, 23213311, 23213322, 23213333, 23231233, 23232113, 23232131, 23232211, 23232212, 23232311, 23232323, 23233212, 23233221, 23233231, 23233232, 23233312, 23233333, 23311233, 23311323, 23312112, 23312121, 23312122, 23312123, 23312131, 23312223, 23312311, 23312312, 23312323, 23313111, 23313133, 23313212, 23313222, 23313232, 23313233, 23313323, 23313333, 23331233, 23331323, 23332112, 23332221, 23332222, 23332223, 23332231, 23332311, 23332323, 23332331, 23333111, 23333123, 23333131, 23333211, 23333212, 23333213, 23333222, 23333223, 23333232, 23333233, 23333311, 23333312, 23333323, 31111233, 31112231, 31112333, 31113131, 31113132, 31113222, 31113323, 31113331, 31113332, 31131233, 31132231, 31132232, 31132333, 31133233, 31133331, 31211131, 31211232, 31212112, 31212212, 31212232, 31212321, 31212323, 31212331, 31212332, 31212333, 31213232, 31213233, 31213323, 31213331, 31213332, 31232231, 31232312, 31232333, 31233221, 31233222, 31233233, 31311231, 31311233, 31311332, 31312113, 31312133, 31312212, 31312222, 31312231, 31312233, 31312323, 31312332, 31312333, 31313111, 31313131, 31313132, 31313133, 31313223, 31313232, 31313233, 31313333, 31331331, 31331333, 31332131, 31332133, 31332232, 31332233, 31332312, 31332322, 31332323, 31332333, 31333233, 31333322, 31333332, 31333333, 32111333, 32112212, 32112313, 32112321, 32113131, 32113232, 32113233, 32131133, 32132232, 32132233, 32132331, 32133111, 32133232, 32133233, 32133331, 32211323, 32212133, 32212231, 32212232, 32212233, 32212321, 32212323, 32212332, 32212333, 32213123, 32213132, 32213231, 32213333, 32232131, 32232322, 32232331, 32232333, 32233222, 32233332, 32311131, 32311323, 32312212, 32312231, 32312233, 32312311, 32312322, 32312323, 32312331, 32312332, 32312333, 32313133, 32313231, 32313232, 32313233, 32313313, 32313332, 32313333, 32332133, 32332223, 32332231, 32332232, 32332322, 32332323, 32332331, 32332332, 32332333, 32333223, 32333232, 32333233, 32333312, 32333323, 32333333, 33113111, 33113211, 33113212, 33113233, 33131333, 33133131, 33133333, 33212213, 33212311, 33212333, 33213211, 33213232, 33213333, 33232233, 33232312, 33232333, 33233131, 33233233, 33233333, 33311231, 33312133, 33312322, 33312333, 33313223, 33313233, 33313323, 33313333, 33331232, 33331233, 33331333, 33332131, 33332133, 33332221, 33332232, 33332233, 33332323, 33332333, 33333123, 33333231, 33333232, 33333233, 33333321, and 33333323,
wherein the reductase domain comprises at least 50% identity to the reductase domain of SEQ ID NO:1, 2 or 3 and wherein the polypeptide has monooxygenase activity.

9. The polypeptide of claim 8, wherein the heme domain is selected from the group consisting of:
21112233, 21112331, 21112333, 21113333, 21212233, 21212333, 21311231, 21311233, 21311311, 21311313, 21311331, 21311333, 21312133, 21312211, 21312213, 21312231, 21312311, 21312313, 21312331, 21312332, 21312333, 21313231, 21313233, 21313313, 21313331, 21313333, 22112233, 22112333, 22212333, 22311233, 22311331, 22311333, 22312231, 22312233, 22312331, 22312333, 22313231, 22313233, 22313331, and 22313333.

10. The polypeptide of claim 8, wherein the heme domain has a CO-binding peak at 450 nm.

11. The polypeptide of claim 8, wherein the 1-10 conservative amino acid substitutions exclude substitutions at residues: (a) 47, 78, 82, 94, 142, 175, 184, 205, 226, 236, 252, 255, 290, 328, and 353 of SEQ ID NO:1; and (b) 48, 79, 83, 95, 143, 176, 185, 206, 227, 238, 254, 257, 292, 330, and 355 of SEQ ID NO:2 or SEQ ID NO:3.

12. The polypeptide of claim 8, 10, or 11, wherein the polypeptide comprises
(1) a Z1 amino acid residue at positions: (a) 47, 82, 142, 205, 236, 252, and 255 of SEQ ID NO:1; (b) 48, 83, 143, 206, 238, 254, and 257 of SEQ ID NO:2 or SEQ ID NO:3;
(2) a Z2 amino acid residue at positions: (a) 94, 175, 184, 290, and 353 of SEQ ID NO:1; (b) 95, 176, 185, 292, and 355 of SEQ ID NO:2 or SEQ ID NO:3;
(3) a Z3 amino acid residue at position: (a) 226 of SEQ ID NO:1; (b) 227 of SEQ ID NO:2 or SEQ ID NO:3; and
(4) a Z4 amino acid residue at positions: (a) 78 and 328 of SEQ ID NO:1; (b) 79 and 330 of SEQ ID NO:2 or SEQ ID NO:3, wherein a Z1 amino acid residue includes glycine (G), asparagine (N), glutamine (Q), serine (S), threonine (T), tyrosine (Y), or cysteine (C). A Z2 amino acid residue includes alanine (A), valine (V), leucine (L), isoleucine (I), proline (P), or methionine (M). A Z3 amino acid residue includes lysine (K), or arginine (R). A Z4 amino acid residue includes tyrosine (Y), phenylalanine (F), tryptophan (W), or histidine (H).

13. A polypeptide having the general structure from N-terminus to C-terminus: (segment 1)-(segment 2)-(segment 3)-(segment 4)-(segment 5)-(segment 6)-(segment 7)-(segment 8)-reductase domain, wherein segment 1 comprises at least 50-100% identity to the sequence of SEQ ID NO:4 (“1”), 5 (“2”), or 6 (“3”); wherein segment 2 comprises at least 50-100% identity to the sequence of SEQ ID NO:7 (“1”), 8 (“2”), or 9 (“3”); wherein segment 3 comprises at least 50-100% identity to the sequence of SEQ ID NO:10 (“1”), 11 (“2”) or 12 (“3”); segment 4 comprises at least 50-100% identity to the sequence of SEQ ID NO:13 (“1”), 14 (“2”), or 15 (“3”); segment 5 comprises at least 50-100% identity to the sequence of SEQ ID NO:16 (“1”), 17 (“2”), or 18 (“3”); segment 6 comprises at least 50-100% identity to the sequence of SEQ ID NO:19 (“1”), 20 (“2”), or 21 (“3”); segment 7 comprises at least 50-100% identity to the sequence of SEQ ID NO:22 (“1”), 23 (“2”), or 24 (“3”); and segment 8 comprises at least 50-100% identity to a sequence of SEQ ID NO:25 (“1”), 26 (“2”), or 27 (“3”), wherein the reductase domain comprises at least 50-100% identity to SEQ ID NO:28,
wherein the segments 1-8 have the general order from N- to C-terminus:
11112212, 11113233, 11113311, 11131313, 11132223, 11132232, 11133231, 11212112, 11212333, 11213133, 11213231, 11232111, 11232232, 11232333, 11311233, 11312233, 11313233, 11313333, 11331312, 11331333, 11332212, 11332233, 11332333, 11333212, 12112333, 12113221, 12211232, 12211333, 12212112, 12212211, 12212212, 12212223, 12212332, 12213212, 12232111, 12232112, 12232232, 12232233, 12232332, 12233112, 12233212, 12313331, 12322333, 12331123, 12331333, 12332223, 12332333, 12333331, 12333333, 13113311, 13213131, 13221231, 13222212, 13233212, 13332333, 13333122, 13333132, 13333211, 13333233, 21111321, 21111323, 21111333, 21112122, 21112123, 21112132, 21112212, 21112222, 21112232, 21112233, 21112311, 21112312, 21112331, 21112332, 21112333, 21113111, 21113112, 21113122, 21113133, 21113211, 21113212, 21113221, 21113223, 21113312, 21113321, 21113322, 21113333, 21131121, 21132112, 21132113, 21132212, 21132222, 21132311, 21132313, 21132321, 21132323, 21133112, 21133113, 21133131, 21133211, 21133222, 21133223, 21133232, 21133233, 21133312, 21133313, 21133321, 21133322, 21133331, 21133332, 21211223, 21211321, 21212111, 21212112, 21212122, 21212123, 21212133, 21212212, 21212213, 21212231, 21212233, 21212321, 21212332, 21212333, 21213121, 21213212, 21213223, 21213231, 21213321, 21213332, 21222112, 21231232, 21231233, 21232112, 21232122, 21232132, 21232212, 21232222, 21232231, 21232232, 21232233, 21232321, 21232322, 21232323, 21232332, 21233111, 21233132, 21233212, 21233221, 21233233, 21233312, 21233321, 21311122, 21311223, 21311231, 21311233, 21311311, 21311313, 21311331, 21311333, 21312111, 21312112, 21312122, 21312123, 21312133, 21312211, 21312213, 21312222, 21312223, 21312231, 21312233, 21312311, 21312313, 21312321, 21312322, 21312323, 21312331, 21312332, 21312333, 21313111, 21313112, 21313122, 21313221, 21313231, 21313233, 21313311, 21313312, 21313313, 21313322, 21313331, 21313333, 21331223, 21331332, 21331333, 21332111, 21332112, 21332113, 21332122, 21332131, 21332212, 21332221, 21332223, 21332231, 21332233, 21332312, 21332322, 21332323, 21332331, 21332332, 21332333, 21333111, 21333122, 21333131, 21333132, 21333211, 21333212, 21333221, 21333223, 21333233, 21333312, 21333321, 22313333, 21333333, 22111223, 22111332, 22112111, 22112131, 22112211, 22112223, 22112233, 22112321, 22112323, 22112331, 22112333, 22113111, 22113211, 22113223, 22113232, 22113233, 22113313, 22113323, 22113332, 22131221, 22132112, 22132113, 22132212, 22132231, 22132233, 22132312, 22132323, 22132331, 22133112, 22133211, 22133212, 22133232, 22133312, 22133322, 22133323, 22212111, 22212123, 22212131, 22212212, 22212232, 22212312, 22212321, 22212322, 22212333, 22213111, 22213112, 22213132, 22213212, 22213222, 22213223, 22213312, 22213321, 22222121, 22231221, 22231223, 22231312, 22231322, 22232111, 22232112, 22232121, 22232122, 22232123, 22232212, 22232222, 22232223, 22232232, 22232233, 22232311, 22232312, 22232322, 22232323, 22232331, 22232333, 22233112, 22233211, 22233212, 22233221, 22233222, 22233223, 22233312, 22233323, 22233332, 22311123, 22311212, 22311231, 22311233, 22311331, 22311333, 22312111, 22312123, 22312132, 22312133, 22312211, 22312221, 22312222, 22312223, 22312231, 22312232, 22312233, 22312311, 22312312, 22312322, 22312331, 22312332, 22312333, 22313122, 22313212, 22313221, 22313222, 22313231, 22313232, 22313233, 22313323, 22313331, 22313332, 22323313, 22331123, 22331133, 22331221, 22331223, 22331323, 22331332, 22332112, 22332113, 22332121, 22332123, 22332132, 22332211, 22332221, 22332222, 22332223, 22332232, 22332233, 22332312, 22332321, 22332322, 22332332, 22333112, 22333122, 22333131, 22333132, 22333133, 22333211, 22333212, 22333221, 22333222, 22333223, 22333231, 22333311, 22333313, 22333321, 22333323, 22333332, 23112213, 23112221, 23112223, 23112233, 23112323, 23112333, 23113111, 23113112, 23113121, 23113131, 23113212, 23113311, 23113312, 23113323, 23113332, 23122212, 23131323, 23132111, 23132121, 23132212, 23132221, 23132232, 23132233, 23132311, 23132322, 23132323, 23133112, 23133113, 23133121, 23133233, 23133311, 23133321, 23133331, 23133333, 23211132, 23212112, 23212211, 23212212, 23212221, 23212222, 23212231, 23212332, 23212333, 23213112, 23213121, 23213123, 23213211, 23213212, 23213223, 23213232, 23213311, 23213322, 23213333, 23231233, 23232113, 23232131, 23232211, 23232212, 23232311, 23232323, 23233212, 23233221, 23233231, 23233232, 23233312, 23233333, 23311233, 23311323, 23312112, 23312121, 23312122, 23312123, 23312131, 23312223, 23312311, 23312312, 23312323, 23313111, 23313133, 23313212, 23313222, 23313232, 23313233, 23313323, 23313333, 23331233, 23331323, 23332112, 23332221, 23332222, 23332223, 23332231, 23332311, 23332323, 23332331, 23333111, 23333123, 23333131, 23333211, 23333212, 23333213, 23333222, 23333223, 23333232, 23333233, 23333311, 23333312, 23333323, 31111233, 31112231, 31112333, 31113131, 31113132, 31113222, 31113323, 31113331, 31113332, 31131233, 31132231, 31132232, 31132333, 31133233, 31133331, 31211131, 31211232, 31212112, 31212212, 31212232, 31212321, 31212323, 31212331, 31212332, 31212333, 31213232, 31213233, 31213323, 31213331, 31213332, 31232231, 31232312, 31232333, 31233221, 31233222, 31233233, 31311231, 31311233, 31311332, 31312113, 31312133, 31312212, 31312222, 31312231, 31312233, 31312323, 31312332, 31312333, 31313111, 31313131, 31313132, 31313133, 31313223, 31313232, 31313233, 31313333, 31331331, 31331333, 31332131, 31332133, 31332232, 31332233, 31332312, 31332322, 31332323, 31332333, 31333233, 31333322, 31333332, 31333333, 32111333, 32112212, 32112313, 32112321, 32113131, 32113232, 32113233, 32131133, 32132232, 32132233, 32132331, 32133111, 32133232, 32133233, 32133331, 32211323, 32212133, 32212231, 32212232, 32212233, 32212321, 32212323, 32212332, 32212333, 32213123, 32213132, 32213231, 32213333, 32232131, 32232322, 32232331, 32232333, 32233222, 32233332, 32311131, 32311323, 32312212, 32312231, 32312233, 32312311, 32312322, 32312323, 32312331, 32312332, 32312333, 32313133, 32313231, 32313232, 32313233, 32313313, 32313332, 32313333, 32332133, 32332223, 32332231, 32332232, 32332322, 32332323, 32332331, 32332332, 32332333, 32333223, 32333232, 32333233, 32333312, 32333323, 32333333, 33113111, 33113211, 33113212, 33113233, 33131333, 33133131, 33133333, 33212213, 33212311, 33212333, 33213211, 33213232, 33213333, 33232233, 33232312, 33232333, 33233131, 33233233, 33233333, 33311231, 33312133, 33312322, 33312333, 33313223, 33313233, 33313323, 33313333, 33331232, 33331233, 33331333, 33332131, 33332133, 33332221, 33332232, 33332233, 33332323, 33332333, 33333123, 33333231, 33333232, 33333233, 33333321, and 33333323,
wherein the polypeptide has monooxygenase activity.

14. The polypeptide of claim 13, wherein the heme domain is selected from the group consisting of:
21112233, 21112331, 21112333, 21113333, 21212233, 21212333, 21311231, 21311233, 21311311, 21311313, 21311331, 21311333, 21312133, 21312211, 21312213, 21312231, 21312311, 21312313, 21312331, 21312332, 21312333, 21313231, 21313233, 21313313, 21313331, 21313333, 22112233, 22112333, 22212333, 22311233, 22311331, 22311333, 22312231, 22312233, 22312331, 22312333, 22313231, 22313233, 22313331, and 22313333.

15. The polypeptide of claim 13, wherein the polypeptide has improved monooxygenase activity compared to a wild-type polypeptide consisting of SEQ ID NO:1, 2, or 3.

16. The polypeptide of claim 13, wherein the substrate specificity of the polypeptide is different compared to the wild-type polypeptide consisting of SEQ ID NO:1, 2, or 3.

17. A polynucleotide encoding a polypeptide of claim 1.

18. The polynucleotide of claim 17, wherein the polynucleotide comprises sequences from each of SEQ ID NO:37, 38, and 39.

19. A polynucleotide encoding a polypeptide of claim 8.

20. A polynucleotide encoding a polypeptide of claim 13.

21. A vector comprising a polynucleotide of claim 17, 19 or 20.

22. A host cell comprising the vector of claim 21.

23. A host cell comprising a polynucleotide of claim 17, 19 or 20.

24. An enzymatic preparation comprising a polypeptide of claim 1, 8 or 13.

25. An enzymatic preparation comprising a polypeptide produced by a host cell of claim 22.

26. An enzymatic preparation comprising a polypeptide produced by a host cell of claim 23.
